(19) United States
(12) Patent Application Publication
(10) Pub. No.: US 2022/0397528 A1
(43) Pub. Date: Dec. 15, 2022

(54) SYSTEMS AND METHODS FOR RAPID, SENSITIVE MULTIPLEX IMMUNOASSAYS

(71) Applicant: The Regents of the University of Michigan, Ann Arbor, MI (US)

(72) Inventors: Katsuo Kurabayashi, Ann Arbor, MI (US); Muneesh Tewari, Ann Arbor, MI (US); Sung Won Choi, Ann Arbor, MI (US); Yujing Song, Ann Arbor, MI (US); Shiuan-Haur Su, Ann Arbor, MI (US)

(21) Appl. No.: 17/776,131

(22) PCT Filed: Nov. 13, 2020
(86) PCT No.: PCT/US2020/060500

§ 371 Date: May 11, 2022

Related U.S. Application Data
(60) Provisional application No. 63/016,758, filed on Apr. 28, 2020.
(60) Provisional application No. 62/936,147, filed on Nov. 15, 2019.

(51) Int. Cl. G01N 21/64; G01N 15/06; G01N 33/557

(52) U.S. Cl. CPC G01N 21/6428; G01N 15/0612; G01N 21/6452; G01N 33/557; G01N 2015/0065

(57) ABSTRACT

The present disclosure provides methods, systems, and kits for detecting molecules in a sample with a pre-equilibrium digital immunoassay. The methods and systems provide means for quantifying molecules in a biological sample of minimal volume in short amounts of time.

### CROSS-REFERENCE TO RELATED APPLICATIONS

This application claims the benefit of U.S. Provisional Application No. 62/936,147, filed Nov. 15, 2019 and U.S. Provisional Application No. 63/016,758, filed Apr. 28, 2020, the contents of each of which are incorporated herein by reference.

### STATEMENT REGARDING FEDERALLY SPONSORED RESEARCH OR DEVELOPMENT

This invention was made with government support under ECCS1708706 and CBET1931905 awarded by the National Science Foundation. The government has certain rights in the invention.

### FIELD

The present disclosure provides systems and methods for detecting a molecule with a pre-equilibrium digital binding assay.

### BACKGROUND

Immunoassays are powerful techniques for biomarker analysis which take advantage of the ability of an antibody to recognize and bind a specific protein existing in a complex mixture of macromolecules. The enzyme-linked immunosorbent assay (ELISA) is the gold standard biomarker detection method widely used in clinical diagnosis because of its high sensitivity and selectivity, but it generally lacks the speed to provide timely data for diagnosis and treatment of acute illnesses.

Digital immunoassays are emerging techniques for biochemical analysis of analytes in low abundance. Their single-molecule sensitivity originates from binary counting of On/Off signals amplified within various types of small sub-volume partitions. Existing digital immunoassay methods also suffer other impediments, including limited multiplexity, long assay incubation time, the inability to deliver a near-bedside result, and increased complexity and cost resulting from bulky optics and fluid handling system. Few studies have implemented digital ELISA (dELISA) assays for protein detection with a platform suited for point-of-care (POC) diagnosis. A recently developed smartphone-connected microfluidic platform for miniaturized dELISA detection achieved a very low limit of detection (LOD) of 0.004-0.007 pg/mL. However, this platform still requires a relatively long sample incubation time, greater than 90 min, thus leading to a total sample-to-answer time of greater than 2 hours. Overall, the conventional digital assays face a similar issue whether they use bulky commercial instrument or a POC platform. Altogether, these limitations pose major obstacles towards fulfilling the promise of biomarker-guided point-of-care (POC) precision medicine in critical care.

### SUMMARY

Disclosed herein are methods for detecting a molecule in a sample comprising: contacting a sample with a capture agent specific for the molecule and a detection agent; incubating the sample with the capture agent and detection agent to form a capture agent-molecule-detection agent complex, wherein the incubating is less than a time necessary for equilibrium conditions to be reached in formation of the complex; and detecting said molecule.

The methods may further comprise separating the capture agent and the capture agent-molecule-detection agent complex from remaining sample and unbound detection agent and isolating each capture agent and capture agent-molecule-detection agent complex into individual locations within a solid support.

Disclosed herein are systems for detecting a molecule in a sample comprising one or more or each of: a capture agent comprising a particle coated with a first probe configured to bind the molecule, a detection agent comprising a second probe configured to bind the molecule, an incubator configured to incubate a sample with the capture agent and detection agent to form a capture agent-molecule-detection agent complex, for a time that is less than a time necessary for equilibrium conditions to be reached in formation of a complex between said capture agent, said detection agent, and said molecule, a solid support, a detector, software configured to determine the presence or absence of the capture agent and the detection agent from the output of the detector and a sample.

Also disclosed herein are reaction mixtures comprising: a stopped incubation mixture of a sample comprising a molecule, a capture agent, a detection agent, and a plurality of capture agent-molecule-detection agent complexes, wherein the stopped mixture is stopped at a time less than a time necessary for equilibrium conditions to be reached in formation of the capture agent-molecule-detection agent complex.

Further disclosed are kits comprising one or more or each of at least one capture agent comprising a particle coated with a first probe configured to bind a molecule of interest, at least one detection agent comprising a second probe configured to bind a same molecule of interest as the capture agent, a detection moiety selected from the group consisting of a dye, a radiolabel, an enzyme, and an enzyme substrate, a labeling agent, a solid support, a detector, software configured to determine the presence or absence of the capture agent and the detection agent from the output of the detector.

Other aspects and embodiments of the disclosure will be apparent in light of the following detailed description and accompanying figures.

### BRIEF DESCRIPTION OF THE DRAWINGS

FIGS. 1A and 1B are diagrams of certain embodiments of capture agents for a dual-plex pre-equilibrium quenching digital ELISA (PEdELISA) assay using 2.8 μm-diameter superparamagnetic beads first dyed with Alexa Fluor (AF) 488 and then conjugated with capture antibodies against TNF-α or MCP-1 (FIG. 1A) and 2.8 μm-diameter non-color superparamagnetic beads conjugated with capture antibodies against IL-6 or Il-2. (FIG. 1B)

FIGS. 1C-1F are images of arrayed microwells for a dual-plex PEdELISA assay in bright field (FIG. 1C). AF 488 channel (FIG. 1D), QuantaRed (Qred) channel (FIG. 1E), and three-channel (bright field+AF 488+Qred) overlay modes (FIG. 1F).

FIG. 1G is a MATLAB code-processed bright field image used to determine the overall bead filling rate with filled (+) and empty (−) wells.

FIG. 1H is a sorted microwell intensity histogram.

FIG. 1I is a graph of bead and microwell counting across 100 arrays of microwells.

FIG. 1J is a MATLAB code-processed two-channel (AF 488+Qred) overlay image for dual-color digital counting (dark green square: AF488-dyed bead, blue circle: non-color bead with Qred emission, light green diamond: AF488-dyed bead with Qred emission).

FIGS. 1K-1L are images showing active Qred “On” microwell spots with the presence of bead-bound analyte molecules (e.g., TNF-α and IL-2) at 100 pg/mL (FIG. 1K), 20 pg/mL (FIG. 1), and 4 pg/mL (FIG. 1M).

FIGS. 2A and 2B are schematics of embodiments of the PEdELISA concepts of instantaneous single-molecule binary counting of pre-equilibrium protein binding events (FIG. 2A) and the two-step ultrafast, dual-plex PEdELISA process for pre-equilibrated assay system (FIG. 2B) The PEdELISA may include a short (15-300 sec) two-color magnetic bead incubation for the formation of antibody-antigen-antibody immune-complexes (Step 1), buffer exchange, quick (30 sec) avidin-HRP labeling (Step 2), and 6-repeated rinsing within 96-well low-retention tubes. The digitization process involves trapping of magnetic beads into on-chip microwell arrays, loading of HRP fluorescence substrate (QuantaRed), and sealing of beads with fluorocarbon oil. Digital signal readout by automated image scanning and counting of fluorescently activated “On”-state microwells. The two-layer (microwell layer, micro-chamber layer) PDMS-based microfluidic detection chip which contains a total of 5.376 million d=3.8 μm microwells can handle 16 samples per scanning. CapAb: capture antibody. DeAb: detection antibody.

FIG. 3 is a diagram of one embodiment of an automated wide-field fluorescence scanning unit suitable for use with the methods and systems disclosed herein. In some embodiments, a digital signal may be read by MATLAB pre-programmed to scan the image and count the fluorescently activated “On”-state microwells and fluorescence encoded magnetic beads.

FIGS. 4A-4C are diagrams of the finite element analysis of biomolecular interactions in the 2-step PEdELISA process. FIG. 4A is a schematic of the theoretical sphere, namely the “reaction volume.” used for modeling work, whose quantity is equal to the total sample volume divided by the number of beads. Reagent mass transport and binding kinetics are considered at the surface of a single magnetic bead placed in its center for half of the geometry due to symmetry. FIG. 4B is Step 1 of the PEdELISA process: complex formation involving the conjugation between target antigen molecules, capture antibodies immobilized on the bead surface, with detection antibodies freely floating in the reaction volume. FIG. 4C is Step 2 of the PEdELISA process: avidin-HRP labeling process involving the conjugation of avidin-HRP with the biotinylated detection antibodies.

FIGS. 4D and 4E are graphs showing the average number of targets (i.e., capture antibody-antigen detection antibody immune-complexes) formed per bead, λ, in the PEdELISA process calculated as a function of the Step 1 incubation time and the analyte concentration at K_{d}=10⁻¹⁰ M (FIG. 4D) and K_{d}=10⁻⁹ M (FIG. 4E). The model predicts that the PEdELISA readout linearly increases with the analyte concentration when λ is small (<0.1). By accounting for the experimentally obtained noise floor, the limit of detection (LOD) value can be determined for a given value of the Step 1 incubation time.

FIG. 4F is a graph showing predicted kinetics of the second step of the PEdELISA process. The fraction of the formation of HRP enzyme-labeled antibody-antigen-antibody immune-complexes is presented for three representative HRP concentrations of 1 pM, 10 pM, and 100 pM.

FIGS. 5A-5D are graphs showing the impact of detection antibody (DeAb) concentration, number of magnetic beads per sample, capture antibody binding site density (Bd), and mass transport on immune complex formation kinetics of PEdELISA assay. The assay signal output is determined by the average number of targets (capture antibody-antigen-detection antibody immune-complexes) formed per bead as a function of the Step 1 incubation time for different antibody-to-antigen on-rate association constant values: kₒₙ=10⁴ M⁻¹s⁻¹ (FIG. 5A), kₒₙ=10⁵ M⁻¹s⁻¹ (FIG. 5B), kₒₙ=10⁶ M⁻¹s⁻¹ (FIG. 5C), and kₒₙ=10⁷ M⁻¹s⁻¹ (FIG. 5D).

FIGS. 6A-6E are PEdELISA assay medium tests for 1×ELISA buffer, 10%, 25%, and 50% fetal bovine serum (FBS) spiked with 100 pg/mL of IL-6 (FIG. 6A), TNF-α (FIG. 6B), MCP-1 (FIG. 6C), and IL-2 (FIG. 6D). FIG. 6E is a graph of the signal-to-noise ratio (SNR) of IL-6, TNF-α, MCP-1, and IL-2 measurements in different media. All the tests were performed with the total assay incubation time of 5 min+30 s. The SNR value represents the ratio of the spike-in signal to the background (negative control) signal. (P>0.05 n=5-8, one-way ANOVA).

FIGS. 7A-7G are graphs of the characterization and optimization of one embodiment of the PEdELISA assay. FIGS. 7A-7D are PEdELISA standard curves for each of the four cytokines: IL-6 (FIG. 7A), MCP-1 (FIG. 7B). TNF-α (FIG. 7C), and IL-2 (FIG. 7D), with the Step 1 incubation time varying as shown and the Step 2 incubation time fixed at 30 sec. Due to the extreme under-labeled nature of the assay, the 15-sec and 30-sec assays were performed by merging the Step 1 and Step 2 process into a single step by mixing all required reagents. The LOD was determined by concentration from the reagent blank's signal+3σ (dotted line). FIGS. 7E and 7F are graphs showing the correlation between PEdELISA and conventional sandwich ELISA tests for the four cytokines using spike-in recombinant proteins in 25% fetal bovine serum: 15-sec PEdELISA incubation time (FIG. 7E) and 300-sec PEdELISA incubation time (FIG. 7F). The ground truth is plotted in dotted line with scattered pre-determined spike-in concentrations. FIG. 7G is a graph showing the theoretical (line) and experimental (scatter) LOD of PEdELISA as a function of the Step 1 incubation time for four cytokines.

FIG. 8A and FIG. 8B are graphs of a dual-plex PEdELISA specificity determined for combinations of TNF and IL-2 (FIG. 8A) and IL-6 and MCP-1 (FIG. 8B) with a 300-sec incubation process. Only one type of cytokine was spiked-in into each medium and signals were measured for both probes in the dual-plex PEdELISA assay. In consideration of the relatively weak antibody-antigen affinity of MCP-1, the spike-in concentration of MCP-1 was set to be 5 times higher than those of the other three cytokines.

FIGS. 9A-9C show the real-time monitoring of CitH3 on living mice (M1-4) with cecal ligation puncture (CLP). FIG. 9A is a diagram of the CLP and mouse blood collection procedures.

FIG. 9B is a graph of the correlation between PEdELISA and conventional sandwich ELISA for the CitH3 assay using a 10% sham mouse serum spiked with recombinant peptide. FIG. 9C is a graph of the longitudinal CitH3 profiles of the four mice: 100%, 75%, 50% ligation, and sham over their lifetimes.

FIGS. 10A-10E show the near-real-time molecular monitoring of multi-cytokines of hematological cancer patients under CAR-T cell therapy. FIG. 10A is a timeline of the hematological cancer patients receiving CAR-T cell therapy. FIG. 10B is a graph showing good agreement (R²=0.96) between 300-sec PEdELISA and ELISA for measurements of selected unknown CAR-T patient samples at different time points for four cytokines. FIGS. 10C-10E are time-series profiles of CRS and CRES grade, IL-6, MCP-1, TNF-α, and IL-2 for Patient A (FIG. OC grade 4 severe CRS), Patient B (FIG. 10D, grade 2 mild CRS) and Patient C (FIG. 10E, grade 0-1 very mild-CRS). Day 0 represents the day of CAR-T cell infusion. Data before Day 0 represents the baseline. The dotted lines represent the time point when anti-cytokine drug tocilizumab (Anti-IL-6R), infliximab (Anti-TNF-α) were administered, as indicated. The shaded region marks the period that the patient was received dexamethasone (corticosteroid). These data do not account for a time lag of a few hours (or occasionally 8-12 hours) between the points of CRS grade recording and blood draw.

FIGS. 11A-11D are group comparisons (non-CRS vs CRS) of the three CAR-T cell therapy patients for IL-6 (FIG. 11A), MCP-1 (FIG. 11B), TNF-α (FIG. 11C), and IL-2 (FIG. 11D). One-way ANOVA comparing means with the Tukey test: IL-6 (P<0.001), MCP-1 (P<0.001) and IL-2 (P=0.0059) levels were significantly higher in CRS condition than in non-CRS condition: TNF-α level was not significant (P=0.142). Data obtained at the time points when CRS-suffering patients were put on steroids or immunosuppressive agents (tocilizumab, infliximab) was included.

FIGS. 12A-12E show one embodiment of the approach for ultrafast highly-multiplexed PEdELISA. FIG. 12A is a concept of microfluidic spatial-spectral encoding: multi-color of magnetic beads with different capture antibodies are pre-patterned into physically separated microarrays to create N_{color}×N_{array} plex. Pre-equilibrium single molecular counting in femtoliter-sized microwells by quenching the reaction time from 15-600 sec. FIG. 12B is one embodiment of a 24-plex CAR-T cytokine panel design. FIG. 12C is one embodiment of a PEdELISA microfluidic chip which includes a patterning layer and a detection layer (24-plex assay version). FIG. 12D is one embodiment of a parallel fluid handling system using the multichannel pipette (sample loading) and syringe pump (on-chip washing). FIG. 12E is one embodiment of a detector: dual-color fluorescence optical scanning unit using an inexpensive CMOS camera.

FIGS. 13A and 13B are images of one embodiment of a low-cost PDMS thin film (FIG. 13A) to glass (FIG. 13B) transfer technique guided by CNC machined alignment jig.

FIG. 14 is a schematic of one embodiment of the architecture of two-color bi-direction convolutional neural network (CNN)-guided signal processing. Multi-image processing includes AF488 channel, Qred channel, and bright-field. The bi-direction CNN contains two neural networks which detect the targets versus defects simultaneously to achieve efficient and accurate digital counting recognition.

FIGS. 15A and 15B are graphs of the performance comparison between CNN and conventional global thresholding image processing on both the Qred channel (FIG. 15A) and AF488 channel (FIG. 15B). The counting error was defined as: Error (%)=(N_{TP}−N_{CNN or Thres})/N_{TP}×100%.

FIG. 16 is a graph of a test of CNN optical cross-talk accuracy using dual-color IL-1α and IL-1β detection by spiking IL-1α:1 ng/mL IL-1:1 ng/mL, IL-1α: 1 ng/mL IL-1β:1 pg/mL, IL-1α:1 pg/mL IL-1β: 1 ng/mL, and IL-1α:1 pg/mL IL-1β:1 pg/mL in 25% fetal bovine serum buffer.

FIG. 17 is graphs of assay specificity test by spiking-in 100 pg/mL of various cytokine in 25% FBS either together (top, left-side graph) or individually, with a blank for FBS without any cytokine (bottom, right-side graph).

FIG. 18 is standard curves from 0.16 pg/mL to 2500 pg/mL cytokine in 25% fetal bovine serum with 5 min and 2 min antigen detection antibody incubation, as indicated, and 1 min enzyme labeling.

FIG. 19 is a graph of the theoretical binding kinetics of bead surface adsorption process. The pre-equilibrium state is determined by the time constant s based on the Langmuir model.

FIG. 20 is a schematic of an exemplary CNN processed PEdELISA microarray analysis. The top shows microfluidic spatial-spectral encoding method used for multiplexing digital immunoassay. Fluorescence color-encoded magnetic beads coated with different capture antibodies are pre-deposited into the array of hexagonal-shaped biosensing patterns in the microfluidic detection channel. The locations of the biosensing patterns are physically separated from each other. This arrangement yields N_{color}×N_{array} measurement combinations determining the assay plexity, Nₚₗₑₓ, where N_{color} is the total number of colors used for encoding beads deposited in each biosensing pattern, and N_{array} is the total number of the arrayed biosensing patterns in each detection channel. In this example, N_{color}=2 (non-fluorescent and Alexa Fluor® 488: AF488) and N_{array}=8. The bottom shows a convolutional neural network-guided image processing algorithm for high throughput and accurate single molecule counting. Two neural networks were run in parallel, reading multicolor fluorescence image data, recognizing target features versus defects, and generating an output mask for post data processing. The brightfield image was analyzed using a Sobel edge detection algorithm. The images were finally overlaid to determine the fraction of enzyme active beads emitting QuantaRed™ signal (Qred+ beads) to total beads for each color label. The unlabeled scale bars are 25 μm.

FIGS. 21A-21C show the PEdELISA immunoassay system employing a PEdELISA microarray chip (FIG. 21A) prepared with a multi-array biosensor layer attached to a bead patterning layer (left) and to a sample loading layer (right), a parallel fluid handling unit (FIG. 21B) using a multichannel pipette for sample loading and a syringe pump for on-chip washing, and a programmed dual-color fluorescence optical scanning setup (FIG. 21C).

FIG. 22 shows exemplary multiplexing of the digital immunoassay by constructing a PEdELISA microarray chip based on the concept of microfluidic spatial-spectral encoding. (Top) Trapping beads into microwells of the arrayed biosensing patterns on the multi-array biosensor layer. Mixtures of fluorescently encoded beads of N_{color} colors are loaded to the microfluidic channels on the bead settling layer. (Bottom) Peeling off the bead settling layer from the multi-array biosensor layer, and attaching the sample loading layer on the multi-array biosensor layer. The 90° orientation of the sample loading channels permits each channel to contain an array of biosensing patters of N_{array} types. Loading serum samples to the channels of the sample detection layer with pipettes. The chip arrangement yields a total of N_{color}×N_{array} plex for the analysis of each sample.

FIG. 23 is a graph of the fraction of AF-488 encoded beads invading the channel to be loaded with noncolor coded beads before and after the bead flushing test assay (P=0.550). On average, only 0.087% of the trapped beads were misplaced in the channel for the both cases. The result was obtained from the fluorescence images of 160 independent microwell sites. The harsh assay conditions with a washing buffer flow rate of 40 uL/min and a duration of 15 min caused negligible physical crosstalk between the beads.

FIGS. 24A and 24B are schematics for the comparison between the global thresholding segmentation, GTS, (FIG. 24A) and the convolutional neural network. CNN, (FIG. 24B) algorithms. In GTS, an optimized global threshold value is predetermined based on the intensity histogram of the image to be processed (shown by the black dash line). The CNN method does not require the predetermination of the threshold value.

FIG. 25 shows an exemplary training process of the dual-pathway convolutional neural network with semantic segmentation. The data library was carefully pre-selected based on 3000 representative images (32×32 pixels) to pre-train the CNN (Left). The images were first labeled based on GTS and then manually modified based on human supervision. The pre-trained CNN was then used to label a new data library which contains around 200 larger images (256×256 pixels) to further improve the feature extraction and classification accuracy (Right).

FIGS. 26A-26E show the image processing by convolutional neural network (CNN) and global thresholding and segmentation (GTS) methods. FIG. 26A is representative images demonstrating false signal counting (red dot: Qred+ microwell, green dot: AF-488-colored bead, yellow dot: recognized spot to be counted). (i) The circle represents an area covered by an aqueous reagent solution that is spread over multiple microwell sites due to poor confinement during the oil sealing process. GTS counts potentially false and unreliable signal spots from the area. CNN removes the area from counting. (ii) Image defocusing causes GTS to merge two signal spots from a pair of the neighboring Qred+ microwells in the circle and to count it as a single signal spot. (iii) Secondary illumination of microwell sites due to optical crosstalk in the circle results in their false counting by GTS. (iv) GTS fails to label and count microwell sites holding dim AF-488-colored beads. Error analysis of CNN and GTS methods on Qred-channel (FIG. 26B) AF488-channel (FIG. 26C) and brightfield images (FIG. 26D). FIG. 26E shows tests assessing the impact of optical crosstalk on the accuracy of CNN and GTS using dual-color IL-1α and IL-1β detection by spiking (i) IL-1α:1 ng/mL IL-1β:1 ng/mL (ii) IL-1α:1 ng/mL IL-1β:1 pg/mL (iii) IL-1a:1 pg/mL IL-1β:1 ng/mL (iv) IL-1α:1 pg/mL IL-1β:1 pg/mL (v) IL-1α:1 pg/mL IL-1β:1 pg/mL assay in single plex for validation. All assays were performed in 25% fetal bovine serum buffer.

FIG. 27A shows an exemplary arrangement of a panel of 14 cytokines for CAR-T cytokine release syndrome detection test. The two cytokines labeled with the black and green fonts on each row were detected in the sample detection channels (1, 2, 3, and 4) vertical to the row using noncolor (black) and AF-488 (green) encoded beads, respectively. FIG. 27B shows the counts of microwells filled with beads and microwells recognized with brightfield images for 60 randomly selected arrayed biosensing patterns (43561 microwells per pattern). The average bead filling rate for the biosensing pattern was 55.1%.

FIGS. 28A and 28B show PEdELISA 14-plex microarray analysis. FIG. 28A shows assay standard curves for 14 cytokines from 0.16 pg/mL to 2500 pg/mL in 25% fetal bovine serum (FBS). FIG. 28B shows the assay specificity test with 25% FBS “all-spike-in,” “single-spike-in,” and “no-spike-in” (negative) samples. The analyte concentration of 500 pg/mL used for spiking FBS is the optimal value to assess both false positive and negative signals.

FIGS. 29A and 29B show graphs of 14-plex cytokine measurements in longitudinal serum samples from CAR-T patients who were diagnosed grade 1-2 CRS (FIG. 29A) or no CRS (FIG. 29B). Day 0 represents the day of CAR-T cell infusion. Data before Day 0 represents the baseline. The shaded region marks the period that the patient was diagnosed with grade 1-2 CRS. For better visualization, the data was organized and separately plotted based on the cytokine level from high to low.

FIGS. 30A-30C shows one embodiment of the PEdELISA assay platform for monitoring CAR-T therapy-associated CRS. FIG. 30A is a diagram outlining the concept of the instantaneous single-molecule binary counting of pre-equilibrium protein binding events. The combination of pre-equilibrium reaction quenching with single-molecule counting can theoretically achieve an assay with a near-zero incubation time without losing linearity. FIG. 3B is a schematic and photo image of one embodiment of the PEdELISA system which comprises a disposable microfluidic chip (inset), an automated fluidic dispensing and mixing module (left), and a 2D inverted fluorescence scanning module (right). FIG. 30C shows the two-step ultrafast, multiplex PEdELISA process for the pre-equilibrated assay system, including 5-min magnetic bead incubation for the formation of antibody-antigen-antibody immune-complexes (Step 1), buffer exchange, 1-min avidin-HRP labeling (Step 2), and 5-min continuous washing using the automated fluidic dispensing module. The PEdELISA chip has 8 circular biosensor patterns formed by a cluster of 66,724 arrayed microwells. Fluorescence color-encoded magnetic beads coated with different capture antibodies (non-fluorescent and Alexa Fluor® 488: AF488) are pre-deposited into each physically separated biosensor pattern. This arrangement can permit multiplex analyte detection with 2 colors×8 patterns=16 plexes. Each chip can quantify up to 16 samples simultaneously per batch run. The digital readout process involves loading of HRP fluorescence substrate (QuantaRed), and sealing of beads with fluorocarbon oil, and signal reading based on automated fluorescence scanning to count fluorescently activated “On”-state microwells. Data analysis is then performed by a convolutional neural network-guided image processing algorithm for high throughput and accurate single-molecule counting.

FIGS. 31A-31N show the rapid longitudinal cytokine profile monitoring of hematological cancer patients under CAR-T cell therapy. FIG. 31A is a graph showing good agreement (R²=0.915) between the PEdELISA and LEGENDplex™ assays found in measurements of 20 CAR-T patient samples at time points randomly picked up for six cytokines. FIG. 31B is a table of the clinical summary of 10 CAR-T patients that includes the maximum CRS score/CRES grade and the number of measurement time points. FIG. 31C is a graph of the distribution of CRS and non-CRS periods (days) during the entire inpatient duration for 10 CAR-T patients. FIG. 31D is a graph of the baseline cytokine levels of CAR-T patients before CAR-T cell infusion. FIGS. 31E-31N are heat maps showing the clinical severity quantified by CRS and CRES grading, the standard scores (Z scores) of CRP and Ferritin levels, and the standard scores (Z scores) of serum cytokine profiles obtained by PEdELISA for 10 CAR-T patients. The grading of CRS, CRES. Hypotension, and Hypoxia was based on the American Society for Transplantation and Cellular Therapy (ASTCT) Consensus Grading. Each standard score (Z score) was calculated based on triplicate measurements of each analyte concentration value. Day 0 represents the day of CAR-T cell infusion. Data before Day 0 represents the baseline. The patients were grouped according to the severity of CRS or neurotoxicity. Data of Patient 06 (grade 4 severe CRS) (FIG. 31E), Data of Patient 02, 08 and 34 (grade 2 mid CRS) (FIGS. 31D and 31F-31H), Data of Patient 05 (grade 3 neurotoxicity) (FIG. 31I), Data of Patient 12, 14, 17, 25, and 33 (grade 0-1 mild or no CRS) (FIG. 31J-31N). Time plots of concentration are additionally shown for IL-6 and TNF-α to provide information on the outcomes of the treatments with tocilizumab (Anti-IL-6R) and infliximab (anti-TNF-α). The green/yellow dotted vertical lines represent the time points of tocilizumab/infliximab dosing. The shadow region in (FIG. 31E) and (FIG. 31I) represents the period in which the patients received dexamethasone.

FIGS. 32A-32M are graphs of the statistical analysis of biomarker data. Comparison of average cytokine levels across all CAR-T patients on days of CRS (i.e., grade >=1) vs. days of no CRS (i.e., grade 0) for IL-1β (FIG. 32A), TNF-α (FIG. 32B), IL-10 (FIG. 32C), IL-6 (FIG. 32D), IL-12 (FIG. 32E), MCP-1 (FIG. 32F). INF-γ (FIG. 32G), IL-2 (FIG. 32H), IL-8 (FIG. 32I), IL-17A (FIG. 32J), CRP (FIG. 32K), and Ferritin (FIG. 32L) (**** P<0.0001, *** P<0.001, ** P<0.01, *P<0.05, and ns P≥0.05). Each error band indicates the mean with SD. “Baseline” represents data from measurement time points before CAR-T cell infusion. The data includes those obtained from the time points at which patients experiencing CRS were put on steroids or immunosuppressive agents (tocilizumab, infliximab), which could affect cytokine biomarker concentrations. IL-6 variations over 4 different phases of tocilizumab treatment combined for three CAR-T patients who received the treatment (FIG. 32M). “On Toci” represents 0-3 days after the tocilizumab administration and “After Toci” represents >3 days after the tocilizumab administration. Statistically significant elevations of IL-6 in serum were temporarily observed in the “On Toci” phase (P<0.01).

FIGS. 33A-33H are graphs of the biomarker responsiveness to the CRS score variation of Patient 06 (grade 4 CRS). Longitudinal plots of (i) the time rate of change of biomarker concentration (Δc/Δt) in black, (ii) the CRS score in light red, and (iii) the time rate of change of the CRS score (ΔCRS/Δt) in light blue for IL-6 (FIG. 33A), MCP-1 (FIG. 33B), INF-γ (FIG. 33C), IL-10 (FIG. 33D), IL-8 (FIG. 33E), IL-2 and TNF-α (FIG. 33F), CRP (FIG. 33G), and ferritin (FIG. 33H). Typically, the time interval was set as Δt=1 day. Δc was calculated using the concentration at a given time point minus the concentration at one previous time point. The vertical dotted lines represent the time points where an “onset” or an abrupt worsening of the CRS symptom was observed for the patient.

### DETAILED DESCRIPTION

The present disclosure provides a method for the detection of a molecule in a sample using an ultrafast assay (e.g., sandwich binding assay) which targets detection of complex formation during an early pre-equilibrium state. Unlike traditional binding assays, this assay allows a reduction in total incubation time from a few hours to a few minutes while still achieving high sensitivity in clinically relevant concentration ranges.

The 2-step PEdELISA assay format was successfully used for the measurement of CitH3 for living septic mouse models with a serum volume as small as 5 μL, and rapid, high-sensitivity, near-real-time multiplex monitoring of CRS relevant circulating cytokines (IL-6, TNF-α, IL-2, and MCP-1) for three hematological cancer patients showing severe and moderate CRS symptoms after CAR-T cell therapy. Time-course biomarker measurement with conventional ELISA or Luminex methods can only be achieved by retrospective tests using banked samples. In contrast, PEdELISA continuously provided real-time data for blood samples freshly collected from mice and human patients with a high time resolution over the most course of the tests (1-5 hr for mice and 24 hr for humans).

The PEdELISA microarray assay simultaneously detected 14 cytokine biomarkers per sample with a clinically relevant dynamic range of pM-nM, and the entire assay process from sample loading to data delivery was completed within 30 min. Blood samples obtained from a CAR-T patient were tested at different time points during the course of the therapy with the short assay turnaround. The longitudinal measurement proved the ability of the assay platform to continuously monitor a large number of cytokine profiles that were rapidly evolving in the circulatory system of a patient manifesting CRS.

With its speed, sensitivity, multiplexing capacity, and sample-sparing capability, the PEdELISA microarray finds use not only in critical care medicine, which is expected to allow the treatment of life-threatening illnesses caused by emerging diseases (e.g., COVID-19) to be timely and tailored to an individual's comprehensive biomarker profiles, but also for clinical researchers to diagnose acute illnesses, determine the optimal dose, frequency, and timing at which drugs are to be administered, thereby providing a new paradigm of individualized critical care of systemic immune disorders and other time-sensitive critical illnesses.

Section headings as used in this section and the entire disclosure herein are merely for organizational purposes and are not intended to be limiting.

### 1. Definitions

The terms “comprise(s),” “include(s),” “having,” “has,” “can,” “contain(s),” and variants thereof, as used herein, are intended to be open-ended transitional phrases, terms, or words that do not preclude the possibility of additional acts or structures. The singular forms “a.” “and” and “the” include plural references unless the context clearly dictates otherwise. The present disclosure also contemplates other embodiments “comprising,” “consisting of” and “consisting essentially of,” the embodiments or elements presented herein, whether explicitly set forth or not.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

Unless otherwise defined herein, scientific and technical terms used in connection with the present disclosure shall have the meanings that are commonly understood by those of ordinary skill in the art. For example, any nomenclatures used in connection with, and techniques of, cell and tissue culture, molecular biology, immunology, microbiology, genetics and protein and nucleic acid chemistry and hybridization described herein are those that are well known and commonly used in the art. The meaning and scope of the terms should be clear, in the event, however of any latent ambiguity, definitions provided herein take precedent over any dictionary or extrinsic definition. Further, unless otherwise required by context, singular terms shall include pluralities and plural terms shall include the singular.

“Biomolecule,” as used herein, includes large macromolecules (or polyanions) such as proteins, carbohydrates, lipids, and nucleic acids, as well as small molecules such as primary metabolites, secondary metabolites, and nucleotides.

“Biomarker,” as used herein, refers to any substance in which its presence, absence, or relative quantity may indicate a particular disease state in a subject. The biomarker includes, but is not limited to, proteins, polypeptides, nucleic acids, small molecules and the like.

“Biological sample,” as used herein, includes biological fluids, including, but not limited to, whole blood, serum, plasma, synovial fluid, cerebrospinal fluid, bronchial lavage, ascites fluid, bone marrow aspirate, pleural effusion, urine, as well as tumor tissue or any other bodily constituent or any tissue culture supernatant that could contain a molecule of interest.

“Isolating,” as used herein, means any process which results in each individual component of a mixture, such as a single capture agent, or a single capture agent-biomolecule-detection agent complex, being isolated such that only one component is in any one location; that location being optically distinct from any other location. The isolation can be accomplished by utilizing a solid support, as described herein.

“Labeling agent.” as used herein, refers to any molecule or compound which facilitates detection by reacting with the detection moiety to produce a detectable reaction product.

“Magnetic bead,” as used herein, refers to so-called magnetic beads, magnetic microbeads, paramagnetic particles, magnetically attractable particles, magnetic spheres, and magnetically responsive particles. These terms are often used interchangeably throughout the field. As such, “magnetic beads” include any of the particles capable of being manipulated in a liquid with the application of a magnetic field. The magnetism of the bead may include paramagnetic, superparamagnetic, ferromagnetic, antiferromagnetic, and ferrimagnetic properties.

“Polynucleotide” or “oligonucleotide” or “nucleic acid,” as used herein, means at least two nucleotides covalently linked together. The polynucleotide may be DNA, both genomic and cDNA, RNA, or a hybrid, where the polynucleotide may contain combinations of deoxyribo- and ribo-nucleotides, and combinations of bases including uracil, adenine, thymine, cytosine, guanine, inosine, xanthine hypoxanthine, isocytosine and isoguanine. Nucleic acids may be obtained by chemical synthesis methods or by recombinant methods. Polynucleotides may be single- or double-stranded or may contain portions of both double stranded and single stranded sequence. The depiction of a single strand also defines the sequence of the complementary strand. Thus, a nucleic acid also encompasses the complementary strand of a depicted single strand. Many variants of a nucleic acid may be used for the same purpose as a given nucleic acid. Thus, a nucleic acid also encompasses substantially identical nucleic acids and complements thereof.

A “peptide” or “polypeptide” is a linked sequence of two or more amino acids linked by peptide bonds. The polypeptide can be natural, synthetic, or a modification or combination of natural and synthetic. Peptides and polypeptides include proteins such as binding proteins, receptors, and antibodies. The proteins may be modified by the addition of sugars, lipids or other moieties not included in the amino acid chain. The terms “polypeptide”, “protein,” and “peptide” are used interchangeably herein.

“Probe,” as used herein, refers to a molecule that binds specifically or selectively to a molecule. The probe may be a nucleic acid, an aptamer, an avimer, receptor-binding ligands, binding peptides, protein, small organic molecules, or a metal ligand. The probe may be an antibody, antibody fragment, a bispecific antibody or other antibody-based molecule or compound designed to bind to a specific biomolecule. The probe may be the same type of molecule as the biomolecule, for example, a protein biomolecule may be bound by a peptide-based probe. Single stranded polynucleotides of complementary sequence may hybridize to form double stranded polynucleotides. The probe may be a different type of molecule from the biomolecule, for example, a polynucleotide probe may bind to a protein biomolecule.

“Separating,” as used herein, means any spatial partitioning of one or more components from the remainder. Separation therefore includes, but is not limited to, fractionation as well as to a specific and selective enrichment, depletion, concentration and/or isolation of certain fractions or analytes contained in a sample.

“Solid support,” as used herein, refers any solid device capable of isolating individual components of a mixture. For example, the solid support may an array with a spatially defined areas in which individual components are isolated by a surface treatment or a magnetized layer. The solid support may have distinct structures (e.g., chambers, sections, wells, or channels) which separate the components, for example, a microfluidic device or a microtiter plate.

Preferred methods and materials are described below, although methods and materials similar or equivalent to those described herein can be used in practice or testing of the present disclosure. All publications, patent applications, patents and other references mentioned herein are incorporated by reference in their entirety. The materials, methods, and examples disclosed herein are illustrative only and not intended to be limiting.

### 2. Methods for Detecting a Molecule

The present disclosure provides methods for detecting a molecule in a sample comprising one or more or each of the steps of: a) providing a mixture of a capture agent and a detection agent; wherein the capture agent comprises a particle (e.g., magnetic bead) coated with a first target configured to bind the molecule, and wherein the detection agent comprises a second target configured to bind the molecule; b) adding the mixture to the sample; c) incubating the sample with the mixture to form a capture agent-molecule-detection agent complex, wherein length of incubating is less than a time necessary for equilibrium conditions to be reached in formation of the complex; d) separating the capture agent and the capture agent-molecule-detection agent complex from the sample and unbound detection agent; e) isolating each capture agent and capture agent-molecule-detection agent complex into individual locations within a solid support; and f) determining the presence or absence of the capture agent and detection agent within each of the individual locations.

The sample includes any composition which comprises the molecule of interest. The sample may be obtained from any source, including bacteria, protozoa, fungi, viruses, organelles, as well higher organisms such as plants or animals, including humans. Samples can be obtained from other sources, including, but not limited to, environmental sources, food products, and forensic samples.

In some embodiments, the sample is a biological sample, including, but not limited to, samples obtained from cells, bodily fluids (e.g., blood, a blood fraction, urine, etc.), or tissue samples by any of a variety of standard techniques. The sample may be, for example, plasma, serum, spinal fluid, lymph fluid, peritoneal fluid, pleural fluid, oral fluid, and external sections of the skin; samples from the respiratory, intestinal genital, and urinary tracts; samples of tears, saliva, blood cells, stem cells, or tumors. Samples may also be obtained from live or dead organisms or from in vitro cultures. Samples comprising cells may require cell lysis before use in the systems and methods disclosed herein.

The total volume of the sample can vary depending on the type of sample and the molecule(s) of interest. In some embodiments, the sample have a volume less than 100 uL, less than 90 uL, less than 80 uL less than 70 uL less than 60 uL less than 50 uL less than 40 uL less than 30 uL less than 20 uL less than 20 uL. In certain embodiments, the sample volume is between 1 and 25 uL. The sample volume may be between 1 and 20 uL, between 1 and 15 uL, between 1 and 10 uL, between 1 and 5 uL, between 5 and 25 uL, between 10 and 25 uL, between 15 and 25 uL, between 20 and 25 uL, between 5 and 20 uL, between 10 and 20 uL, between 15 and 20 uL, between 5 and 15 uL, 5 between and 10 uL, or between 10 and 15 uL.

The sample may be diluted prior to use in the systems and methods disclosed herein. The sample may be diluted about 1-fold, about 2-fold, about 3-fold, about 4-fold, about 5-fold, about 6-fold, about 10-fold, or greater, prior to use.

Many potential target molecules may be detected and, optionally, quantified using methods and systems of the present invention. Any target molecule that may be bound by capture and detection probes can be detected by the methods described herein. For example, the molecule may include: hormones, phosphoproteins, glycoproteins, lipoproteins, immunoglobulins, growth factors, cytokines, metabolites, small molecules, or small molecules drugs. In some embodiments, the molecule is a polypeptide, a polysaccharide, a polynucleotide, a lipid, a metabolite, a drug, or a combination thereof.

In certain embodiments, the molecule is a biomarker. The biomarker may be any substance in which its presence, absence, or relative quantity in a subject may indicate a particular disease or stage of disease. Biomarkers have been linked to a number of diseases such as, cancer, diabetes, multiple sclerosis, neurodegenerative disorders, stroke, etc. Examples of commonly measured biomarkers in humans include proteins (e.g., cytokines, metabolic enzymes, cell cycle enzymes, cytoskeletal protein, autoantibodies, growth factors, and neuropeptides), hormones (e.g., steroid hormones, dehydroepiandrosterone (DHEA), estrogen, vasopressin, cholesterol, adrenalin, cortisol, and cortisone), metabolites (e.g., alcohol, lactic acid, lactate, urea, and creatinine), and small molecules (e.g., vitamins, glucose, penicillin, and hydrogen peroxide). In exemplary embodiments, the biomarker is a protein biomarker, e.g. a cytokine.

a. Adding a Mixture of Capture and Detection Agents to the Sample

The methods comprise providing a mixture of a capture agent and a detection agent and adding the mixture to the sample.

The capture agent may comprise a magnetic bead or other particle or solid surface coated with a first probe configured to bind the molecule. The magnetic bead may include different labels or detection chemistries, including for example, fluorescent, chemiluminescent, bioluminescent, or isotopic labels. In some embodiments, the magnetic bead is a fluorescent magnetic bead. In some embodiments, the magnetic bead is densely coated with the first probe. The average number of probes per particle may range from 1.0-6.0×10⁵ probes/particle.

The detection agent may comprise a second probe configured to bind the same molecule as the capture agent.

The nature of the first and second probes will depend on the type of target molecule. For example, when the target molecule is a protein, the first and second probes may comprise proteins, particularly antibodies or fragments thereof, other proteins, peptides or small molecules. If the target molecule is a nucleic acid, the probes may be a nucleic acid binding protein or a complementary nucleic acid, if the target molecule is a single-stranded nucleic acid. When the target molecule is a carbohydrate, the first and second probes may include, for example, antibodies, aptamers, lectins, and selectins. Suitable target molecule/probe pairs can include, but are not limited to, antibodies/antigens, receptors/ligands, proteins/nucleic acid, nucleic acids/nucleic acids, enzymes/substrates or inhibitors, carbohydrates (including glycoproteins and glycolipids)/lectins or selectins, proteins/proteins, and proteins/small molecules. In some embodiments, the first probe and the second probe are independently selected from a protein, a peptide, a nucleic acid, a carbohydrate, a small molecule, and a ligand. In exemplary embodiments, the first probe is an antibody. In exemplary embodiments, the second probe is an antibody.

The first and second probes are configured to bind the same target molecule. In some embodiments, the first probe and the second probe are configured to bind different locations within the target molecule.

The detection agent may further comprise a detection moiety selected from the group consisting of a dye, a radiolabel, an enzyme, and an enzyme substrate. In some embodiments, the detection moiety is a fluorescent dye. In some embodiments, the detection moiety is an enzyme or enzyme substrate. In certain embodiments, the enzyme is beta-galactosidase, alkaline phosphatase or horseradish peroxidase.

The methods and systems described herein may be used to detect two or more target molecules. For detection of two or more target molecules, each target molecule uniquely binds to a capture agent/detection agent pair, such that the capture agent/detection agent pair does not bind any of the other two or more target molecules in the sample. When utilizing multiple capture agent/detection agent pairs it is important to select capture agents, detection agents, and detection moieties that facilitate individual measurement of different components so as to be able to accurately determine the presence or absence of the detection agent and capture agent.

b. Incubating the Sample with the Mixture

In some embodiments, the methods further include incubating the sample with the mixture to form a capture agent-molecule-detection agent complex. The length of the incubation is less than the time necessary for equilibrium condition to be reached in formation of the capture agent-molecule-detection agent complex. Equilibrium conditions of a binding reaction exist when the association reaction is balanced by the dissociation reaction, such that the total number of binding complexes remains constant. However, at the onset of incubating two binding components or under pre-equilibrium conditions, the association reaction is vastly favored because there are no or few binding complexes present. Therefore, the length of the incubation is during the time period wherein the association reaction of the molecule with the capture agent is favored and dominating the binding interaction. Current methods utilize much longer incubation times to target measurement of the binding interaction under equilibrium conditions. The measurement of pre-equilibrium conditions was unexpected and surprising.

The length of pre-equilibrium conditions of a binding reaction may be determined using a simple time constant based on ideal Langmuir binding curves. The Langmuir adsorption model is:

$$\frac{d [ AbL ]}{dt} = {k}_{on} [ L ] ( {[ Ab ]}_{0} - [ AbL ] ) - {k}_{off} [ AbL ] \qquad ( 1 )$$

where [L] is volume ligand concentration, [Ab]₀ is initial surface antibody concentration, [AbL] is the surface concentration of ligand-antibody complex, and kₒₙ and k_{off} are respectively the on-rate and off-rate constants.

Assuming the solution ligand concentration [L]=c₀=Constant (not changing with time, perfect mass transfer), (1) can be solved as:

$$BR = \frac{[ AbL ]}{{[ Ab ]}_{0}} = \frac{{c}_{0}}{{c}_{0} + {K}_{d}} [ 1 - {e}^{- {( {k}_{on} {c}_{0} + {k}_{off} )}^{t}} ] \qquad ( 2 )$$

where BR is the surface binding ratio. By letting t→∞, the asymptote is represented as

$${BR}_{∞} = \frac{{c}_{0}}{{c}_{0} + {K}_{d}} .$$

In an increasing system, the time constant t is the time for the system to reach (1−e⁻¹)≈63.2% of its final asymptotic value. Therefore:

$$τ = \frac{1}{{k}_{on} {c}_{0} + {k}_{off}} = \frac{{k}_{off}^{- 1}}{1 + {c}_{0} / {K}_{d}} \qquad ( 3 )$$

At pre-equilibrium: t<τ.

For example, the typical dissociation constant K_{d} for antibody to antigen is 1 nM, with kₒₙ≈10⁶ M⁻¹s⁻¹ and k_{off}≤10⁻³ s⁻¹ and the clinically relevant cytokine detection concentration c₀ is generally from 10 fM to 0.1 nM. Therefore, the time constant τ would be estimated to be around 1000 sec (16.7 min).

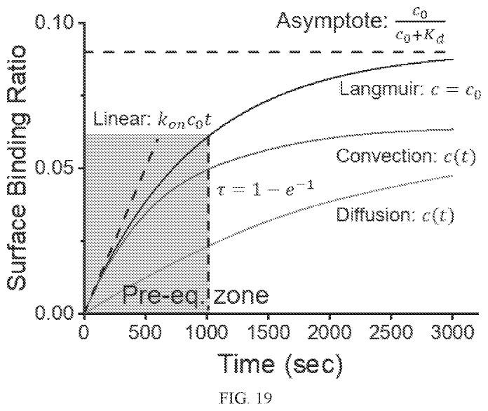

In reality, the solution ligand concentration [L] is not a constant but decreases over time c(t) as the bio-reaction progresses and it takes time for the target molecules to diffuse. As such, the binding curve will look like the diffusion line as shown in FIG. 19 (solved by a numerical solver COMSOL) or somewhere in the middle of the convection and diffusion line, rather than the Langmuir line. Nonetheless, the pre-equilibrium zone determined by a simple time constant based on the ideal Langmuir binding curves will apply to the diffusion and convections lines as well.

In some embodiments, the length of the incubation is between 15 seconds and 45 minutes. The length of the incubation may be greater than 15 seconds, 30 second, 45 seconds, 60 seconds, 2 minutes, 5 minutes, 10 minutes, 15 minutes, 20 minutes, 25 minutes, 30 minutes, 35 minutes, or 40 minutes. The length of the incubation may be less than 45 minutes, 40 minutes, 35 minutes, 30 minutes, 25 minutes, 20 minutes, 15 minutes, 10 minutes, 5 minutes, 2 minutes, 60 second, 45 second, or 30 second. In certain embodiments, the length of the incubation is between 15 seconds and 600 seconds. In exemplary embodiments, the length of the incubation is between 15 seconds and 300 seconds.

The total amount of capture agent and detection agent added to the sample will vary depending on the abundance of the molecule of interest in the sample and the volume of the sample. In some embodiments, the total number of capture agents (e.g., magnetic bead+first probe) incubated with the sample is from 10⁵-10⁶. In some embodiments, the total concentration of detection agents is between 0.25-1 μg/mL.

In some embodiments, the sample may be mixed during the incubation by stirring, shaking, rotating, swirling, vortexing, or other appropriate means based on the incubation vessel.

Following the incubation, capture agent and capture agent-molecule-detection agent complexes are separated from the remainder of the sample and any unbound detection agent. The separation essentially quenches or stops the binding reaction prior to reaching equilibrium conditions due to the removal of one of the components of the reaction.

The separation may utilize any means necessary or useful that allows selective removal of the sample and unbound detection agent. For example, where the capture agent comprises a magnetic bead, this may be done using a magnet to partition the capture agent and capture agent-molecule-detection agent complexes from the other components. Other methods may include filtration, affinity separation, and/or centrifugation.

At least one washing step may be carried out following the separation. Preferred wash solutions are those that do not change the configuration of the capture agent, molecule, or detection agent and do not disrupt the binding interactions in the capture agent-molecule-detection agent complexes.

c. Isolating Each Capture Agent and Capture Agent-Molecule-Detection Agent Complex

In some embodiments, the methods further include isolating each capture agent and each capture agent-molecule-detection agent complex, or subsets of such agents and complexes, into individual locations within a solid support. The isolation may result in the individual locations in the solid support being populated with a capture agent, a capture agent-molecule-detection agent complex, or neither species.

The solid support may be smooth, having a substantially planar surface, or it may contain a variety of structures such as wells, grooves, depressions, channels, elevations, chambers, or the like, in which each capture agent or capture agent-molecule-detection agent complex is isolated. The solid support may be a microfluidic device comprising a series of microchannels which isolate the individual capture agents or capture agent-molecule-detection agent complexes. The solid support may be a multi-well plate comprising a vast number of wells which isolate the individual capture agents or capture agent-molecule-detection agent complexes. The solid surface may be a magnetic array such that individual regions of magnetism result in the isolation of each capture agent or capture agent-molecule-detection agent complex on a substantially planar surface. In some embodiments, the solid support is a microplate or microfluidic device.

The solid support may be composed of any of a wide variety of materials, for example, polymers, plastics, resins, polysaccharides, silica or silica-based materials, carbon, metals, inorganic glasses, membranes, or any combinations thereof. The solid support material may be treated, coated, modified, printed or derivatized using polymers, chemicals to impart desired properties or functionalities to the array support surface. Preferred solid support material may be compatible with the range of conditions encountered during the assay including salt concentrations and solvents, be stable under the application of magnetic fields, and be optically transparent.

The solid supports may be those commercially available or formed using common methods including, but not limited to, film deposition processes, such as spin coating and chemical vapor deposition, laser fabrication or photolithographic techniques, wet chemical or plasma etching methods, and/or molding or casting.

The solid support may be sealed prior to detection to prevent evaporation or migration of the contents in the individual locations during the remainder of the analysis. The solid support may be sealed after the isolation or after the optional addition of a labeling agent. Sealing methods include, for example, overcoating the top surface of the solid support with a sealing fluid (e.g. a non-aqueous fluid, such as oil) or using a sealing tape to isolate each individual location.

d. Determining the Presence/Absence of a Capture Agent or Detection Agent

In some embodiments, the methods further include determining the presence or absence of the capture agent and detection agent within each of the individual locations.

Determining the presence or absence of the capture agent may comprise detection of the magnetic bead. In some embodiments, this may be done with a brightfield detector such that the presence of the bead at each location is identified. In the cases where the magnetic bead comprises a detectable tag or label, such as a fluorescence tag, a fluorescence microscope with a camera or other detector may be used.

The presence of the detection agent may indicate the presence of the molecule of interest such that that location comprises a capture agent-molecule-detection agent complex. Determining the presence of absence of the detection agent may be done directly or indirectly. In the case of direct detection, the detection agent may comprise a detection moiety that may be directly measured. For example, if the detection moiety includes a dye or radioactive isotope, presence of the detection agent may be determined with optical detection of the dye, either fluorescent or visible detection, or infrared spectroscopy or autoradiography, respectively. In the case of indirect detection, the detection agent may comprise a detection moiety that reacts with a labeling agent to form a detectable reaction product.

In some embodiments, the detection agent comprises a detection moiety which can be directly detected. In some embodiments, the method may further comprise adding a labeling agent to the separated capture agents and the capture agent-molecule-detection agent complexes, such that the labeling agent reacts with the detection moiety to produce a reaction product. Thus, in some embodiments, determining the presence or absence of the detection agent comprises measurement of the reaction product. The labeling agent may be added before or after isolation of the capture agent/capture agent-molecule-detection agent complexes into individual locations, preferably after isolation.

In some embodiments, the labeling agent is a substrate for an enzyme included in the capture agent such that upon contact with the enzyme converts the labeling agent into a chromogenic, fluorogenic, or chemiluminescent reaction product, which is detectable. In some embodiments, the labeling agent is an enzyme and the substrate is included in the capture agent. Any known chromogenic, fluorogenic, or chemiluminescent labeling agents may be selected for conversion by many different enzymes. In exemplary embodiments, the enzyme may be beta-galactosidase, horseradish peroxidase, or alkaline phosphatase. The substrate can respectively be an eta-galactosidase, horseradish peroxidase, or alkaline phosphatase well known in the art that are labeled or create a measurable signal upon enzymatic reaction, including, but not limited to: 3.3′,5,5′-tetramethylbenzidine, 3,3′-diaminobenzidine, 2,2′-azino-bis(3-ethylbenzothiazoline-6-sulphonic acid), p-nitrophenyl phosphate, 2,2′-azinobis [3-ethylbenzothiazoline-6-sulfonic acid]-diammonium salt, o-phenylenediamine dihydrochloride, or other enhanced fluorescent or chemiluminescent derivatives thereof.

The detection methods and type of detector employed depend on the nature of the capture agent, detection agent, or labeling agent reaction products. Non-limiting examples of detection methods include optical imaging (fluorescence and visible), Raman scattering, spectroscopy (e.g., infrared, atomic, fluorescence or visible spectroscopies), absorbance, circular dichroism, electron microscopies (e.g., scanning electron microscopy (SEM), x-ray photoelectron microscopy (XPS)), light scattering, optical interferometry and other methods known in the art based on measuring changes in refractive index, diffraction, absorption, and fluorescence technologies.

In some embodiments, the detector may comprise more than one light source and/or a plurality of filters to adjust the wavelength and/or intensity of the light source. In some embodiments, the detector may also include a microscope (light or fluorescent) and/or a camera to capture the detection of the optical output of the detection method. The camera maybe a CCD (charge-coupled device) or CMOS (complementary metal-oxide-semiconductor) camera or similar camera known in the art. By using a camera with an electrical image converter, such as a CCD or CMOS chip, high local resolution can be achieved. The detector may also include a computer or controller used to control the light source, the filters, and/or execute any imaging processing software.

The detector may capture the optical output of the entire solid support at one time. Or the detector may move throughout the solid support during the detection to survey the entire solid support for the presence/absence of capture agents and detection agents.

A measure of the concentration of the molecule may be based on the number and/or fraction of locations determined to contain a capture agent and a detection agent. The concentration may be based on the fraction of locations comprising both the capture agent and the detection agent compared to locations comprising only the capture agent. The concentration may be based on the fraction of locations comprising both the capture agent and the detection agent compared to total locations.

In some embodiments, the methods further comprise quantifying the concentration of the molecule based on the fraction of locations comprising both the capture agent and the detection agent to locations comprising only the capture agent. Following the determination of the presence/absence of capture agent and detection agent for each location, the data for the locations may be analyzed by software including an algorithm based on a Poisson distribution, to determine the average number of binding complexes per bead. The software may remove false positives, the presence of imaging defects, contamination and aggregations of capture agent or detection agent in any of the locations. For example, the algorithm may apply a binary “Off” or “On” state to each of the location based on the presence of the capture agent only, or the presence of the detection agent, respectively. Then, the fraction of the “On” states may be correlated with molecule concentration, for example, from a standard or calibration curve for the molecule of interest.

### 3. Systems for Detecting a Molecule

The present disclosure provides systems (e.g., reagents, computer software, instruments, etc.) for detecting at least one molecule in a sample. In some embodiments, the systems comprise at least one capture agent comprising a particle (e.g., magnetic bead) coated with a first probe configured to bind one of the at least one molecule and at least one detection agent comprising a second probe configured to bind the one of the at least one molecule.

The capture agent may comprise a magnetic bead coated with a first probe configured to bind the molecule. The magnetic bead may include different labels or detection chemistries, including for example, fluorescent, chemiluminescent, bioluminescent, or isotopic labels. In some embodiments, the magnetic bead is a fluorescent magnetic bead. The detection agent may comprise a second probe configured to bind the same molecule as the capture agent.

The nature of the first and second probes will depend on the type of target molecule. For example, when the target molecule is a protein, the first and second probes may comprise proteins, particularly antibodies or fragments thereof, other proteins, peptides or small molecules. If the target molecule is a nucleic acid, the probes may be a nucleic acid binding protein or a complementary nucleic acid, if the target molecule is a single-stranded nucleic acid. When the target molecule is a carbohydrate, the first and second probes may include, for example, antibodies, lectins, and selectins. Suitable target molecule/probe pairs can include, but are not limited to, antibodies/antigens, receptors/ligands, proteins/nucleic acid, nucleic acids/nucleic acids, enzymes/substrates or inhibitors, carbohydrates (including glycoproteins and glycolipids)/lectins or selectins, proteins/proteins, and proteins/small molecules. In some embodiments, the first probe and the second probe are independently selected from a protein, a peptide, a nucleic acid, a carbohydrate, a small molecule, and a ligand. In exemplary embodiments, the first probe is an antibody. In exemplary embodiments, the second probe is an antibody.

The detection agent may further comprise a detection moiety selected from the group consisting of a dye, a radiolabel, an enzyme, and an enzyme substrate. In some embodiments, the detection moiety is a fluorescent dye. In some embodiments, the detection moiety is an enzyme or enzyme substrate. In certain embodiments, the enzyme is beta-galactosidase, alkaline phosphatase or horseradish peroxidase.

The systems may further comprise a labeling agent. The labeling agent reacts with the detection moiety to produce a reaction product.

The systems may also comprise a sample (e.g., positive and/or negative control samples), a solid support, a detector, and/or software configured to determine the presence or absence of the capture agent and the detection agent from the output of the detector. In some embodiments, an instrument is provided that automates one or more of the steps of the methods described herein. For example, in some embodiment, the instrument comprises software that controls incubations time to, for example, start and stop reactions such that the pre-equilibrium incubations times described herein are achieved.

The sample includes any composition which comprises the molecule of interest. The sample may be obtained from any source, including bacteria, protozoa, fungi, viruses, organelles, as well higher organisms such as plants or animals, including humans. Samples can be obtained from other sources, including, but not limited to environmental sources, food products, and forensic samples. In some embodiments, the sample is a biological sample.

The solid support may be smooth, having a substantially planar surface, or it may contain a variety of structures such as wells, grooves, depressions, channels, elevations, chambers, or the like, in which each capture agent or capture agent-molecule-detection agent complex is isolated. The solid support may be a microfluidic device comprising a series of microchannels which isolate the individual capture agents or capture agent-molecule-detection agent complexes. The solid support may be a multi-well plate comprising a vast number of wells which isolate the individual capture agents or capture agent-molecule-detection agent complexes. The solid surface may be a magnetic array such that individual regions of magnetism result in the isolation of each capture agent or capture agent-molecule-detection agent complex on a substantially planar surface. In some embodiments, the solid support is a microplate or microfluidic device.

The type of detector employed depends on the nature of the capture agent, detection agent, or labeling agent reaction products. Non-limiting examples of detection methods include optical imaging (fluorescence and visible), Raman scattering, spectroscopy (e.g., infrared, atomic, fluorescence or visible spectroscopies), absorbance, circular dichroism, electron microscopies (e.g., scanning electron microscopy (SEM), x-ray photoelectron microscopy (XPS)), light scattering, optical interferometry and other methods known in the art based on measuring changes in refractive index, diffraction, absorption, and fluorescence technologies.

The detector may comprise more than one light source and/or a plurality of filters to adjust the wavelength and/or intensity of the light source. The detector may also include a microscope (light or fluorescent) and/or a camera to capture the detection of the optical output of the detection method. The camera maybe a CCD or CMOS camera or similar camera known in the art. By using a camera with an electrical image converter, such as a CCD or CMOS chip, high local resolution can be achieved. The detector may also include a computer or controller used to control the light source, the filters, and/or execute any imaging processing software. 30. In some embodiments, the detector comprises an optical microscope, fluorescence microscope, a fluorometer, a spectrophotometer, a camera, or a combination thereof.

The software may be supplied with the systems in any electronic form such as a computer readable device, an internet download, or a web-based portal. The software may be integrated with the detector to not only determine the presence or absence of the capture agent and the detection agent from the output of the detector, and/or a sample but also control the detector components. The software may allow a user to view results in real-time, review results of previous samples, and view reports. The software may output data in the forms of images, graphs, charts, or raw values. The software may also be capable of calculating statistics and making comparisons between data sets.

The systems can also comprise instructions for using the components of the systems. The instructions are relevant materials or methodologies pertaining to the systems. The materials may include any combination of the following: background information, list of components and their availability information (purchase information, etc.), brief or detailed protocols for using the systems, trouble-shooting, references, technical support, and any other related documents. Instructions can be supplied with the systems or as a separate member component, either as a paper form or an electronic form which may be supplied on computer readable memory device or downloaded from an internet website, or as recorded presentation.

The system may further include reagents, computer software, instruments, etc. for obtaining, processing, or preparing a sample. For example, the system may include instruments or devices for taking a sample from a patient (e.g. finger pricks, needles, syringes, and the like), sample separation or pre-processing devices (e.g. plasma separation (apheresis machines), filtration devices, centrifuges, and the like), or extraction or sample stabilizing or separation buffers. In some embodiments, the instruments for obtaining, processing, or preparing a sample may be integrated into any of the devices described above within the system.

It is understood that the disclosed systems can be employed in connection with the disclosed methods.

### 4. Reaction Mixtures

The present disclosure provides reaction mixtures. The reaction mixtures may comprise a stopped incubation mixture of a sample comprising a molecule, a capture agent, a detection agent, and a plurality of capture agent-molecule-detection agent complexes, wherein the stopped mixture is stopped at a time less than a time necessary for equilibrium conditions to be reached in formation of the capture agent-molecule-detection agent complex.

The sample includes any composition which comprises the molecule of interest. The sample may be obtained from any source, including bacteria, protozoa, fungi, viruses, organelles, as well higher organisms such as plants or animals, including humans. Samples can be obtained from other sources, including, but not limited to environmental sources, food products, and forensic samples.

In some embodiments, the sample is a biological sample, including, but not limited to, samples obtained from cells, bodily fluids (e.g., blood, a blood fraction, urine, etc.), or tissue samples by any of a variety of standard techniques. The sample may be, for example, plasma, serum, spinal fluid, lymph fluid, peritoneal fluid, pleural fluid, oral fluid, and external sections of the skin; samples from the respiratory, intestinal genital, and urinary tracts; samples of tears, saliva, blood cells, stem cells, or tumors. Samples may also be obtained from live or dead organisms or from in vitro cultures. Samples comprising cells may require cell lysis before use in the systems and methods disclosed herein.

The capture agent may comprise a magnetic bead or other particle or solid surface coated with a first probe configured to bind the molecule. The magnetic bead may include different labels or detection chemistries, including for example, fluorescent, chemiluminescent, bioluminescent, or isotopic labels. In some embodiments, the magnetic bead is a fluorescent magnetic bead. In some embodiments, the magnetic bead is densely coated with the first probe. The average number of probes per particle may range from 1.0-6.0×10⁵ probes/particle.

The detection agent may comprise a second probe configured to bind the same molecule as the capture agent.

The nature of the first and second probes will depend on the type of target molecule. For example, when the target molecule is a protein, the first and second probes may comprise proteins, particularly antibodies or fragments thereof, other proteins, peptides or small molecules. If the target molecule is a nucleic acid, the probes may be a nucleic acid binding protein or a complementary nucleic acid, if the target molecule is a single-stranded nucleic acid. When the target molecule is a carbohydrate, the first and second probes may include, for example, antibodies, aptamers, lectins, and selectins. Suitable target molecule/probe pairs can include, but are not limited to, antibodies/antigens, receptors/ligands, proteins/nucleic acid, nucleic acids/nucleic acids, enzymes/substrates or inhibitors, carbohydrates (including glycoproteins and glycolipids)/lectins or selectins, proteins/proteins, and proteins/small molecules. In some embodiments, the first probe and the second probe are independently selected from the group consisting of a protein, a peptide, a nucleic acid, a carbohydrate, a small molecule, a ligand, and any combination thereof.

### 5. Kits for Detecting a Molecule

The present disclosure provides kits for detecting a molecule.

The kits may comprise one or more or each of at least one capture agent comprising a particle coated with a first probe configured to bind a molecule of interest, at least one detection agent comprising a second probe configured to bind a same molecule of interest as the capture agent, a detection moiety selected from the group consisting of a dye, a radiolabel, an enzyme, and an enzyme substrate, a labeling agent, a solid support, a detector, software configured to determine the presence or absence of the capture agent and the detection agent from the output of the detector.

The kits may further include reagents, computer software, instruments, etc. for obtaining, processing, or preparing a sample. For example, the kits may include instruments or devices for taking a sample from a patient (e.g. finger pricks, needles, syringes, and the like), sample separation or pre-processing devices (e.g. plasma separation (apheresis machines), filtration devices, centrifuges, and the like), or extraction or sample stabilizing or separation buffers. In some embodiments, the instruments for obtaining, processing, or preparing a sample may be integrated into any of the other components of the kits as described above.

Individual member components of the kits may be physically packaged together or separately. The kits can also comprise instructions for using the components of the kit. The instructions are relevant materials or methodologies pertaining to the kit. The materials may include any combination of the following: background information, list of components and their availability information (purchase information, etc.), brief or detailed protocols for using the system, trouble-shooting, references, technical support, and any other related documents. Instructions can be supplied with the kit or as a separate member component, either as a paper form or an electronic form which may be supplied on computer readable memory device or downloaded from an internet website, or as recorded presentation.

It is understood that the disclosed kits can be employed in connection with the disclosed methods.

### 6. Spatial-Spectral Encoding

The present disclosure provides devices, systems and methods for spatial-spectral encoding. Spatial-spectral encoding is a process in which multiple capture probes are patterned into physically separated microarrays or solid supports (e.g. magnetic beads) labeled with multicolor on a single detectable chip or substrate.

In some embodiments, the devices and methods comprise one or more or each of: a solid support, a sample patterning component and a sample detection component. In some embodiments, the systems and methods further comprise a detector, software, capture agent and detection agent. Illustrative embodiments of each of these components, and their use in the methods is described below.

a. Solid Support

In some embodiments, the devices and methods comprise a solid support as described herein. The solid support may comprise individual locations configured to isolate a molecule of interest. The solid support may contain a variety of structures such as wells, grooves, depressions, channels, elevations, chambers, or the like, in which to isolate a molecule of interest. The solid support may be composed of any of a wide variety of materials, for example, polymers, plastics, resins, polysaccharides, silica or silica-based materials, carbon, metals, inorganic glasses, membranes, or any combinations thereof. The solid support material may be treated, coated, modified, printed or derivatized using polymers, chemicals to impart desired properties or functionalities to the array support surface. Preferred solid support material may be compatible with the range of conditions encountered during the assay including salt concentrations and solvents, be stable under the application of magnetic fields, and be optically transparent with minimum auto-fluoresce background.

The solid support may be commercially available or formed using common methods including, but not limited to, film deposition processes, such as spin coating and chemical vapor deposition, laser fabrication or photolithographic techniques, wet chemical or plasma etching methods, and/or molding or casting.

b. Sample Patterning & Sample Detection Components

In some embodiments, the devices and methods may comprise a sample patterning component and a sample detection component. The sample patterning component and the sample detection component each comprise a plurality of parallel fluid handling channels. In some embodiments, each fluid handling channel in the sample patterning component and the sample detection component is independent from the adjacent fluid handling channels. In some embodiments, each of the fluid handling channels is configured to receive a different fluid sample. In some embodiments, each fluid handling channel comprises an individual inlet and outlet, such that individual solutions or samples can be loaded into the each of the fluid handling channels. In some embodiments, each of the fluid handling channels in the sample detection component is configured to receive the same sample.

Each fluid handling channel is in fluid communication with a portion of the individual locations in the solid support. In some embodiments, the fluid handling channels of the sample patterning component are perpendicular to the fluid handling channels of the sample detection component. In some embodiments, the portion of individual locations in fluid communication with a single fluid handling channel of the sample patterning component is also in fluid communication with each fluid handling channel of the sample detection component. In some embodiments, the portions of individual locations exposed to a first parallel fluid handling channel, are exposed to each of the samples loaded into each fluid handling channel of the sample detection component. In some embodiments, the portions of individual locations exposed to a first parallel fluid handling channel, are exposed to only select samples loaded into the fluid handling channels of the sample detection component.

The sample patterning component and the sample detection component may be composed of any of a wide variety of materials, for example, polymers, plastics, resins, polysaccharides, silica or silica-based materials, carbon, metals, inorganic glasses, membranes, or any combinations thereof. The material may be treated, coated, modified, printed or derivatized using polymers, chemicals to impart desired properties or functionalities to the array support surface. The sample patterning component and the sample detection component may be formed using common methods including, but not limited to, film deposition processes, such as spin coating and chemical vapor deposition, laser fabrication or photolithographic techniques, wet chemical or plasma etching methods, and/or molding or casting.

In some embodiments, a capture agent pool is loaded into each fluid handling channel of a sample patterning component and each capture agent pool is isolated to a portion of the individual locations in the solid support. A capture agent pool comprises at least one capture agent, as defined herein. In some embodiments, a capture agent pool comprises at least one, at least two, at least three, or at least four capture agents. In some embodiments, the capture agents from each capture agent pool may be the same or different. In some embodiments, at least two, at least three, or at least four capture agents capture agents loaded into each fluid handling channel are the same. In some embodiments, at least two, at least three, or at least four capture agents capture agents loaded into each fluid handling channel are different. In some embodiments, each capture agent from a capture agent pool is isolated in an individual location.

In some embodiments, a sample, as described herein, is loaded into each fluid handling channel of a sample detection component and each sample is isolated to a portion of the individual locations in the solid support. In some embodiments, the sample loaded into each fluid handling channel is the same. In some embodiments, the sample loaded into each fluid handling channel is different.

In some embodiments, the sample patterning component and a sample detection component are interchangeably attached to the solid support. In some embodiments, the sample patterning component is attached to the solid support to facilitate loading of the capture agent pool. In some embodiments, the sample patterning component is removed from the solid support and a sample detection component is attached to the support to facilitate loading of the sample.

In some embodiments, the sample incubates with the capture agent in each individual location to form a capture agent-molecule complex.

c. Detector

In some embodiments, the devices and methods comprise a detector.

The detection methods and type of detector employed depend on the nature of the capture agent, detection agent, or labeling agent reaction products. Non-limiting examples of detection methods include optical imaging (fluorescence and visible), Raman scattering, spectroscopy (e.g., infrared, atomic, fluorescence or visible spectroscopies), absorbance, circular dichroism, electron microscopies (e.g., scanning electron microscopy (SEM), x-ray photoelectron microscopy (XPS)), light scattering, optical interferometry and other methods known in the art based on measuring changes in refractive index, diffraction, absorption, and fluorescence technologies.

In some embodiments, the detector may comprise more than one light source and/or a plurality of filters to adjust the wavelength and/or intensity of the light source. In some embodiments, the detector may also include a microscope (light or fluorescent) and/or a camera to capture the detection of the optical output of the detection method. The camera maybe a CCD (charge-coupled device) or CMOS (complementary metal-oxide-semiconductor) camera or similar camera known in the art. By using a camera with an electrical image converter, such as a CCD or CMOS chip, high local resolution can be achieved. The detector may also include a computer or controller used to control the light source, the filters, and/or execute any imaging processing software.

The detector may capture the optical output of the entire solid support at one time. Or the detector may move throughout the solid support during the detection to survey the entire solid support for the presence/absence of capture agents and detection agents.

In some embodiments, prior to detection, the capture agent-molecule complex is contacted with a detection agent, as described herein. In some embodiments, the detector detects the presence of the capture agent, the detection agent, or a combination thereof. In some embodiments, the detector detects the presence or absence of capture agent and detection agent at each individual location in the solid support with the same detector.

The type of detector employed depends on the nature of the capture agent, detection agent, or labeling agent reaction products. Non-limiting examples of detection methods include optical imaging (fluorescence and visible), Raman scattering, spectroscopy (e.g., infrared, atomic, fluorescence or visible spectroscopies), absorbance, circular dichroism, electron microscopies (e.g., scanning electron microscopy (SEM), x-ray photoelectron microscopy (XPS)), light scattering, optical interferometry and other methods known in the art based on measuring changes in refractive index, diffraction, absorption, and fluorescence technologies.

The detector may comprise more than one light source and/or a plurality of filters to adjust the wavelength and/or intensity of the light source. The detector may also include a microscope (light or fluorescent) and/or a camera to capture the detection of the optical output of the detection method. The camera maybe a CCD or CMOS camera or similar camera known in the art. By using a camera with an electrical image converter, such as a CCD or CMOS chip, high local resolution can be achieved.

d. Software

In some embodiments, the devices and methods comprise software configured to spatially separate the identifiable individual locations with the solid support and capable of correlating the output of the detector with the presence or absence of at least one of the plurality of molecules of interest.

In some embodiments, the software comprises a convolutional neural network (CNN) algorithm which take input image(s), classifies and differentiates the various aspects/objects from one another and assign importance to various aspects/objects in the image. For example, the software may analyze detector images based on two different fluorescent signals to determine which individual locations comprise one or both of the fluorescent signals. In addition, the software may also analyze a detector brightfield image, to calculate the total number of individual locations with and without capture agent-complexes and then calculate the percentage of each of those which comprises a capture agent-molecule-detection agent complex based on the fluorescent images.

A measure of the concentration of the molecule may be based on the number and/or fraction of locations determined to contain a capture agent and a detection agent. The concentration may be based on the fraction of locations comprising both the capture agent and the detection agent compared to locations comprising only the capture agent. The concentration may be based on the fraction of locations comprising both the capture agent and the detection agent compared to total locations. In some embodiments, the methods further comprise quantifying the concentration of the molecule based on the fraction of locations comprising both the capture agent and the detection agent to locations comprising only the capture agent.

The software may remove false positives, the presence of imaging defects, contamination and aggregations of capture agent or detection agent in any of the locations. For example, the algorithm may apply a binary “Off” or “On” state to each of the location based on the presence of the capture agent only, or the presence of the detection agent, respectively. Then, the fraction of the “On” states may be correlated with molecule concentration, for example, from a standard or calibration curve for the molecule of interest.

In some embodiments, the algorithm may run two neural networks in parallel for two detection pathways. For example, one neural network may be for assay targets (e.g. microwells, beads, and fluorescence signals) and the other may be for false positives, imaging defects, contamination and aggregations of capture agent or detection agent.

The software may be supplied in any electronic form such as a computer readable device, an internet download, or a web-based portal. The software may be integrated with the detector to not only determine the presence or absence of the capture agent and the detection agent from the output of the detector, and/or a sample but also control the detector components. The software may allow a user to view results in real-time, review results of previous samples, and view reports. The software may output data in the forms of images, graphs, charts, or raw values. The software may also be capable of calculating statistics and making comparisons between data sets.

### 7. Examples

### Materials and Methods

Materials. The mouse CitH3 capture antibody was generated by ProMab Biotechnologies, Inc. (Richmond, Calif., USA). CitH3 detection antibody and HRP-conjugated secondary antibody were purchased from Abeam and Jackson ImmunoResearch. Human IL-6 capture and biotinylated detection antibodies were purchased from BioLegend. Human TNF-α, IL-2, and MCP-1 assay were developed based on uncoated ELISA kits (including capture antibody, biotinylated detection antibody, and avidin-HRP) from Invitrogen. Dynabeads, 2.7 μm-diameter carboxylic acid, and epoxy-linked superparamagnetic beads, avidin-HRP, QuantaRed™, an enhanced chemifluorescent HRP substrate, Alexa Fluor™ 488 Hydrazide, EDC (1-ethyl-3-(3-dimethylaminopropyl) carbodiimide hydrochloride), Sulfo-NHS (Sulfo-N-hydroxysulfosuccinimide), MES (2-(N-morpholino) ethanesulfonic acid) buffered saline, bovine serum albumin (BSA), TBS StartingBlock T20 blocking buffer, and PBS SuperBlock blocking buffer were obtained from Thermo Fisher Scientific. Human IL-6, TNF-α, IL-2, IL-8, IL-13 capture, and biotinylated detection antibody pairs from Invitrogen™, and IL-1α, IL-1β, IL-10, IL-12, IL-15, IL-17A, IFN-γ, GM-CSF and MCP-1 from BioLegend. Phosphate buffered saline (PBS) was also obtained from Gibcor™, Sylgard™ 184 clear polydimethylsiloxane (PDMS) from Dow Corning, and Fluorocarbon oil (Novec™ 7500) from 3M™.

Finite Element Analysis of Transient Digital Assay. The commercial FEA software COMSOL 5.4 Multiphysics was used to model the 2-step PEdELISA process involving molecular transport and bead surface reaction. Several model assumptions were made based on experimental conditions. First, it was assumed that the magnetic beads were evenly distributed in the buffer solution by the orbital shaker mixing during the incubation process. As a result, the model only considered the half of a single bead surface within the “reaction volume,” which is scaled by the sample volume divided by the number of the beads used. The cytokine diffusion profile was evaluated using the transient mass convection and diffusion equation as:

$$\frac{δ c}{δ t} = D \cdot {∇}^{2} c - μ \cdot ∇ c + R \qquad ( 4 )$$

where c is the concentration, the convection term μ·∇c was omitted, and the value of the diffusion coefficient D was adjusted to reflect the mass transport under active mixing. The first step of the PEdELISA process was modeled by considering the simultaneous reactions between the capture antibody (Ab₁), antigen ligand (L), and the detection antibody (Ab₂). The derived kinetics equations using Langmuir isotherm are given as:

$${\frac{d [ {Ab}_{1} L ]}{dt} = {k}_{on 1} ( {Ab}_{1} ]}_{0} - [ {Ab}_{1} L ] ) [ L ] - {k}_{off 1} [ {Ab}_{1} L ] \qquad ( 5 )$$

$${\frac{d [ {Ab}_{2} L ]}{dt} = {k}_{on 2} ( {Ab}_{2} ]}_{0} - [ {Ab}_{2} L ] ) [ L ] - {k}_{off 2} [ {Ab}_{2} L ] \qquad ( 6 )$$

$${{\frac{d [ {Ab}_{2} {LAb}_{1} ]}{dt} = {k}_{on 1} ( {Ab}_{1} ]}_{0} - [ {Ab}_{1} L ] ) [ {Ab}_{2} L ] + {k}_{on 2} ( {Ab}_{2} ]}_{0} - [ \text{} {Ab}_{2} L ] [ {Ab}_{1} L ] - ( {k}_{off 1} + {k}_{off 2} [ {Ab}_{1} {LAb}_{2} ] \qquad ( 7 )$$

where kₒₙ and k_{off} are the association/dissociation constants, and [ ] represents the concentration or surface density of the three agents. For simplicity, the affinity of Ab₁ to L was assumed to be the same as the affinity of Ab₂ to L (kₒₙ₁=kₒₙ₂, k_{off1}=k_{off2}). For the second step labeling process, the avidin-HRP conjugate and the immune-complex, Ab₁LAb₂, were modeled as the “free ligand” and the surface immobilized capture agent, respectively. The kinetic equation for this process is given as:

$${\frac{d [ {Ab}_{2} {LAb}_{1} HRP ]}{dt} = {k}_{on 3} ( {Ab}_{1} {LAb}_{2} ]}_{0} - [ {Ab}_{1} {LAb}_{2} HRP ] ) [ avidinHRP ] - {k}_{off 3} [ {Ab}_{1} {LAb}_{2} HRP ] \qquad ( 8 )$$

Finally, to translate the molecular binding events into the digital assay readout. Poisson distribution equation (9) was used,

λ=−ln(1−Pₚₒₛᵢₜᵢᵥₑ) (9)

where Ppositive is the fraction of fluorescence-activated “On” beads to the entire beads and λ is the mean expectation value, which represents the average number of immune-complexes per bead.

Device Fabrication and Assembly. The microwell structure and the microfluidic channel were fabricated in poly-dimethylsiloxane (PDMS, Dow Corning Sylgard 184) using the standard soft lithography technique. Firstly, two silicon molds, one for the microwell structure with a thickness of 4 μm (SU-8 2005, Micro-Chem), the other for the microfluidic channel with a thickness around 100 mm (SU-8 2050, Micro-Chem), were fabricated by photolithography. Secondly, a precursor of PDMS prepared at a 10:1 base-to-curing agent mass ratio was spin-coated onto the microwell silicon mold (300 rpm, 1 min) and poured over the microfluidic channel mold with a thickness around 4 mm. Both of the molds were left on the flat surface overnight and then cured in an oven at 60° C. for 2 h. The surface of the thin-film PDMS microwell layer cured on the silicon mold wafer was treated by oxygen plasma. The film was aligned using a custom-machined aluminum jig and bonded onto a pre-cleaned 75×50 mm glass slide, and finally removed from the silicon wafer. The PDMS microfluidic channel layer was cut and peeled off its silicon mold and punched manually to form its inlet and outlet. After second oxygen plasma treatment, the top surface of the thin-film PDMS microwell layer was aligned with and bonded to the PDMS microfluidic channel layer. Finally, the entire chip was briefly baked at 60° C. and stored at room temperature before use.

Antibody Conjugation to Magnetic Beads. The non-color encoded magnetic beads were prepared by conjugating epoxy-linked Dynabeads with the capture antibody molecules at a mass ratio of 6 μg (antibody): 1 mg (bead). The Alexa Fluor™ 488 (AF488) encoded magnetic beads were prepared by first labeling carboxylic acid-linked Dynabeads with AF 488 Hydrazide dye and then by conjugating the beads with capture antibody at a mass ratio of 12 pg (antibody): 1 mg (bead) using standard EDC/sulfo-NHS chemistry. Briefly, carboxylic acid-linked Dynabeads were first labeled with AF 488 dye, and then conjugated with the capture antibody molecules as follows: 100 μL of a bead stock solution (30 mg beads/mL) was washed with 25 mM MES buffer at pH=5 for two times, mixed with 100 μL of a 1 mg/mL EDC solution and 100 μL of a 1.13 mg/mL sulfo-NHS solution (25 mM MES buffer), and then incubated at room temperature on an orbital shaker at 1000 rpm for 30 min. Then, the beads were washed two times with the MES buffer and mixed with a 1 μg/mL AF488 hydrazide solution for 30 min. Then, the beads were washed 5 times with 0.5 mL PBS-T (0.1% Tween20) solution, resuspended in 300 μL of a PBS-T (0.05% Tween20) solution, and transferred into a new polypropylene tube. The AF488-encoded beads were washed two times with the MES buffer, reactivated with 100 μL of a 50 mg/mL EDC solution and 100 μL of a 50 mg/mL sulfo-NHS solution for 30 min, and then rinsed two times with the MES buffer. A 100 μL capture antibody solution was prepared and mixed with the activated beads at a mass ratio of 12 μg (antibody): 1 mg (bead) for 2 h at room temperature. Then the beads were separated and washed 4 times with 0.5 mL PBS-T (0.1% Tween20) solution and stored at 10 mg beads/mL in PBS-T (0.05% T20+0.1% BSA+0.01% Sodium Azide) solution wrapped with aluminum foil at 4° C. No significant degradation was observed within the 3-month usage.

Mouse CLP Preparation and Sample Collection. Sepsis was induced by cecum ligation puncture (CLP) in male mice with age between 8-12-week-old. The peritoneal cavity was opened under inhaled isoflurane anesthesia. Cecum was eviscerated, ligated below the ileocecal valve using a 5-0 silk suture at three different points (50%, 75%, and 100%), and punctured through (two holes) with a 21-ga needle. The punctured cecum was squeezed to expel a small amount of fecal material and returned to the peritoneal cavity. The abdominal incision was closed in two layers with 4-0 silk suture. The Sham mouse was handled in the same manner, except that the cecum was not ligated and punctured. Around 15 μL blood was drawn through the tail vein every 4-5 hours after CLP. The blood was allowed to clot by leaving it undisturbed at room temperature for 30 minutes. The clot was then removed by centrifuging at 2000×g for 15 minutes in a refrigerated centrifuge. The supernatant serum was used for the following PEdELISA assay. This protocol was approved by the University of Michigan. All surgery was performed under anesthesia, and all efforts were made to minimize suffering.

Patient Blood Sample Collection and Preparation. Subjects undergoing CAR-T therapy were recruited and samples collected with informed consent for each subject. Control samples were obtained from healthy volunteers with informed consent. All blood samples were collected on-site at the University of Michigan Medical School Hospital. Venous blood was collected for serum into a vacutainer containing no anticoagulant. Blood samples were then transported to the lab, allowed to clot for at least 30 minutes at room temperature, and processed for serum isolation. Samples were centrifuged at 1,200×g for 15 minutes at room temperature. Serum was then removed by pipette and aliquoted into 2 mL screw cap tubes. Serum aliquots were then transported fresh on wet ice for the PEdELISA assay or banked at −80° C.

PEdELISA Assay. The capture antibody beads were first incubated with the TBS StartingBlock buffer (0.05% Tween20) for 30 min to block the beads surface and quench all the unreacted groups. Then the beads were washed once with a PBS-T buffer and divided into 96-well reaction tubes so that each tube has approximately 8×10⁵ beads. The samples were diluted by the ELISA dilution buffer (1% BSA, 0.05% Tween20). The dilution ratio for CitH3 is 1:4. The dilution ratio for IL-2 and TNF-α is 1:2 due to their low abundance in serum and the dilution ratio for IL-6 and MCP-1 is 1:4 or 1:8 based on the potentially high level under severe CRS condition. The recombinant standards were diluted by an ELISA dilution buffer spiked with 25% fetal bovine serum. The diluted samples were temporarily kept on wet ice until use. In the two-step assay protocol, a mixture of 10 μL of the sample or standard and 10 μL of a biotinylated detection antibody (0.25 μg/mL) solution was loaded to the tube and incubated with the magnetic beads for a period of 60 sec to 300 sec. After a quick buffer exchange (1×PBS-T, 0.1% Tween20), the beads were then incubated with 40 μL of the avidin-HRP solution for 30 sec. After washing in a 2×PBS-T (0.1% Tween20) buffer solution 6 times, they were resuspended in 11 μL of a 1×PBS-T (0.1% Tween20) buffer solution. 10 μL of the bead solution was loaded into the premade microfluidic chip, which contains 16 separate channels for different samples. Each channel was then loaded with 20 μL of the enhanced chemifluorescent HRP substrate QuantaRed solution and subsequently sealed with 20 μL of fluorinated oil (HFE-7500, 3M).

For the 14-plex PEdELISA assay, all assay reagents were prepared in 96-well plate low retention tubes and kept on ice until use. The reagent preparation involved preparing a mixture of biotinylated detection antibody (up to 14 cytokines for CAR-T study) in carrier protein buffer (0.1% BSA, 0.02% Sodium Azide) and storing it at 4° C. and preparing an Avidin-HRP solution in a superblock buffer at 100 pM. For the PEdELISA chip calibration, a mixture of recombinant proteins was prepared in 25% fetal bovine serum (standard solution), which was 5× serially diluted from 2.5 ng/mL to 0.16 pg/mL. Prior to the assay, patient serum samples (5 uL) were diluted two times with PBS (5 uL) to prepare a sample solution. As the first step of the assay, the sample solution (10 μL) and the biotinylated detection antibody solution (10 μL) were mixed to form a sample mixture, the 5 titrated standard solutions (10 μL) and the biotinylated detection antibody solution (10 μL) were mixed to form standard mixtures. The sample and standard mixtures were loaded into the detection channels in parallel and incubated the chip for 300 sec. The signals obtained from the standard mixtures were used for calibrating the biosensors of the chip. The microfluidic channels were then washed with PBS-T (0.1% Tween20) at 20 uL/min by syringe pump for 2 min. 40 μL of the avidin-HRP solution was then loaded into the channel and incubate for 1 min. The chip was washed again with PBS-T (0.1% Tween20) at 20 uL/min for 10 min. 30 μL of the enhanced chemifluorescent HRP substrate QuantaRed solution was loaded into the channels and subsequently sealed with 35 μL of fluorinated oil (HFE-7500, 3M).

After sealing with oil, the inlets and outlets of the channels were covered by glass coverslips to prevent evaporation during the imaging process. A programmable motorized fluorescence optical microscopy system was used to scan the image of the bead-filed microwell arrays on the microfluidic chip, identify the bead type (non-color vs. AF488 dyed), and detect the enzyme-substrate reaction activity. This system is composed of Nikon Ti-S fluorescence microscope, a programmable motorized stage (ProScan III), a halogen lamp fluorescence illumination source, a SONY full-frame CMOS camera (α7iii), and a custom machined stage holder. The motorized stage was pre-programmed to follow the designated path to scan the entire chip. The image process took about 20 see to scan each channel (1 sample/channel, total 16 channels), following 3 sequential steps: 1. Scan the QuantaRed channel (532 nm/585 nm, excitation/emission) 2. Scan the AF488 channel (495 nm/519 nm, excitation/emission) 3. Scan the brightfield.

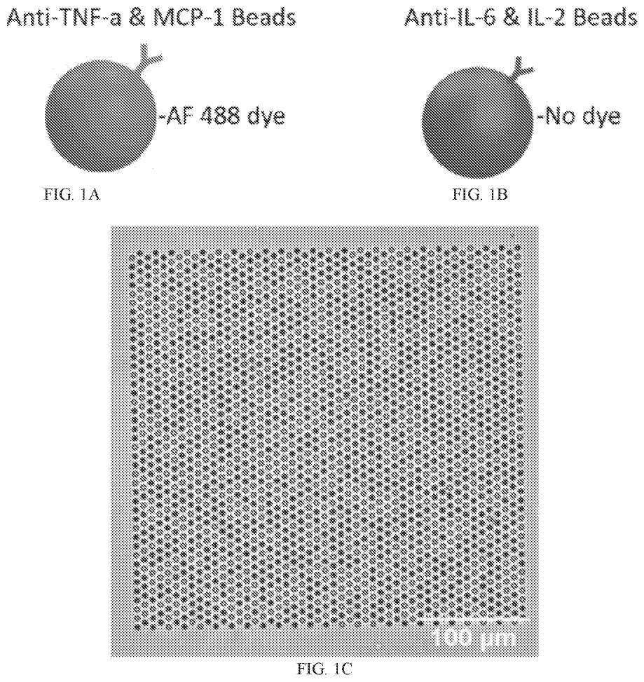

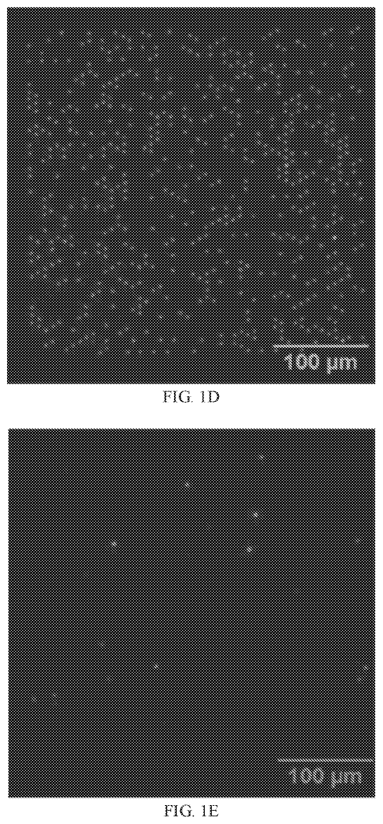

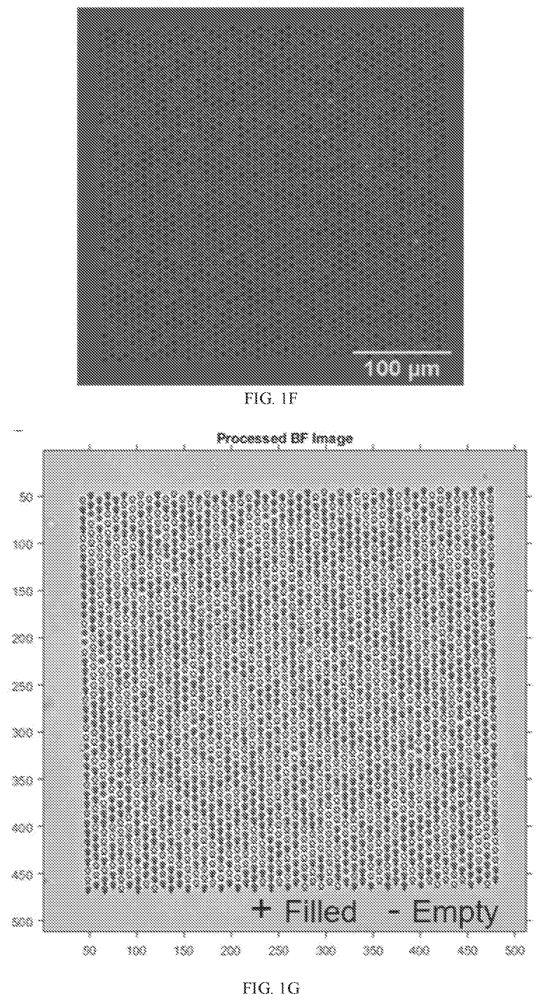

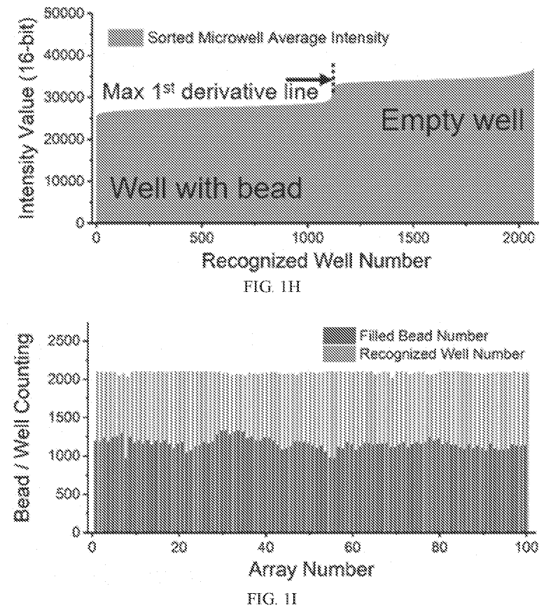

Data Analysis and Inage Processing. In the digital immunoassay, statistical analysis of the fraction of the fluorescence-activated “On” beads to the entire beads across 336,000 femtoliter-sized microwells per channel determined the analyte concentration value. A custom image processing MATLAB code, with or without coupling with convolutional neural network, was used to analyze scanned microwell-array images automatically with high speed and accuracy (FIG. 1).

Briefly, the code simultaneously captures the images from all of the AF488 (bead encoding dye), QuantaRed (labeling dye), and Brightfield channels and superimposes them. Then, it counts the numbers of the “On” and “Off” states for the two types of beads (AF488-encoded and non-color types) trapped in the microwells from the superimposed images. The code includes algorithms to avoid counting aggregated beads and to eliminate signals from false positives, imaging defects, and large fluorescence contaminations. The fraction of the “On” states were correlated with the analyte concentration from the standard curve.

A MATLAB code, without coupling, was used to analyze the scanned images using the following 4-step algorithm. Step 1: The code simultaneously reads all of the bright field (FIG. 1C), Alexa Fluor (AF) 488 (FIG. 1D), and QuantaRed (Qred) (FIG. 1E) images and first identifies the locations of AF488-dyed beads based on fluorescence intensity thresholding (the method of image segmentation by creating binary images). Sub-algorithms identifying the area and signal intensity of beads are used to remove counts of defects like clusters of aggregated beads. If a defect is identified by a pre-defined value of aggregation severity, it is removed from the counts for the Qred and bright field images (similar in step 2 and 3).

Step 2: The Qred image is analyzed based on fluorescence intensity thresholding, size-based circle detection, and morphological dilation and erosion to identify the locations of all the enzyme active “On” (or Qred “On”) microwells. Sub-algorithms identifying the total number, areas, signal intensities, inter-distances, and image boundaries of arrayed microwells are used to eliminate all false positives, image defects, and large fluorescence contaminations. Signal crosstalk is an issue uniquely found in the Qred image analysis. It is a type of false-positive counting that often happens when a microwell is so bright that a few of its nearest neighboring microwells in the hexagonal array arrangement are also brightened up to exceed the threshold intensity. In this case, these neighboring microwells are falsely counted as “On” signals even though they are not actually enzyme active. To mitigate this issue, a distance pattern recognition algorithm was applied, which first identified all the bright spots with their 6 nearest neighbors and then performed a second-round intensity check (high threshold) to determine if their neighboring microwells are true or false positives.

Step 3: The bright field image is analyzed to identify the areas of microwells using edge and pattern recognition algorithms based on the Sobel edge detection methods. Then, the microwell brightness intensity is averaged over the identified area and its values are sorted for the entire arrayed microwells in the bright field image (FIG. 1H). This microwell intensity sorting effectively helps divide the microwells into two distinct groups: those that contain (+) or do not contain (−) beads. The separation line was determined by the maximum intensity slope (first derivative) of the sorted intensity values. Sub-algorithms are subsequently used to eliminate local image areas with poor bead-to-well contrast, air bubbles, and large dust. Step 3 has intrinsic uncertainties due to image quality variances in focus adjustment during the image scanning process. To suppress error due to these uncertainties, the counting process is repeated and averaged over the repeats. For example, in FIG. 1, the original design of each array of microwells contains 2,100 microwell locations but the MATLAB program indicated that the number of recognized microwells is 2,071. Among them, 1,114 wells were identified to have beads inside. The numbers of counted beads and microwells were obtained and averaged across 100 arrays of microwells to determine the overall bead filling rate for counting (FIG. 1I).

Step 4: Finally, the code overlays the local images of the recognized AF488 positive beads on top of the Qred image to determine the numbers of Qred “On” microwells with and without an AF488-dyed bead inside (FIG. 1J). Considering a potential misalignment in the image overlay process, each Qred “On” microwell was marked with a blue circle 1.4 times larger than its original size. Finally, dual-plex cytokine detection was achieved by determining the fractional population of the enzyme active Qred “On” microwells to the bead-filled (+) microwells for both the AF488 dyed and noncolor bead types. FIGS. 1K-1M show Qred image snapshots for a mixture sample of TNF-α and IL-2 both at a concentration ranging from 4 pg/mL to 100 pg/mL.

In addition, a MATLAB code coupled with convolutional neural network was also used. In brief, three types of raw images were collected: 1. Red fluorescence channel (Qred) 2. Green fluorescence channel (AF488) 3. Brightfield channel. The first two channels were sent directly to the bi-direction CNN to classify the Qred+ microwells, AF488+ beads, image defects, and background. Then the Qred+ and AF488+ targets were segmented out as the output mask and the defects were removed. The bright-field image is analyzed using the Sobel edge detection methods to determine the overall beads filling rate. After post-image processing, the code overlaid the three images to determine the numbers of Qred “On” microwells with and without an AF488-dyed bead inside. The fractional population of the enzyme active Qred “On” microwells to the bead-filled microwells for both the AF488 dyed (AF+Qred+%) and non-color bead types (AF−Qred+%) is directly proportional to the two different biomarker concentrations to be determined.

Statistics. Experiments were performed 3 times (in independent tests) to obtain the error bar. Due to the extreme low sample volume (<7 μL) obtained from the CLP mouse at each time point, the CitH3 PEdELISA assay was performed with no repeat. Either duplicate or triplicate PEdELISA measurement was performed for the CAR-T patient sample at a single time point of the near-real-time cytokine profile monitoring test. Conventional ELISA and LEGENDPlex multiplex assays were conducted with no repeat for a few selected time points of the banked serum samples or in duplicate for banked serum samples collected at 20 selected time points. Pearson's R-value was used to quantify the PEdELISA to ELISA/LEGENDPlex correlations. Group differences were tested using either a one-way ANOVA and comparing means with the Tukey test or an unpaired, two-tailed t-test with equal variance. A p-value of <0.05 was considered to be statistically significant. A standard score (Z score) for the parameter x was given as Z=(x−μ)/SD, where μ is the mean and SD is the standard deviation.

### Example 1

### Rationale and Design of the PEdELISA Platform

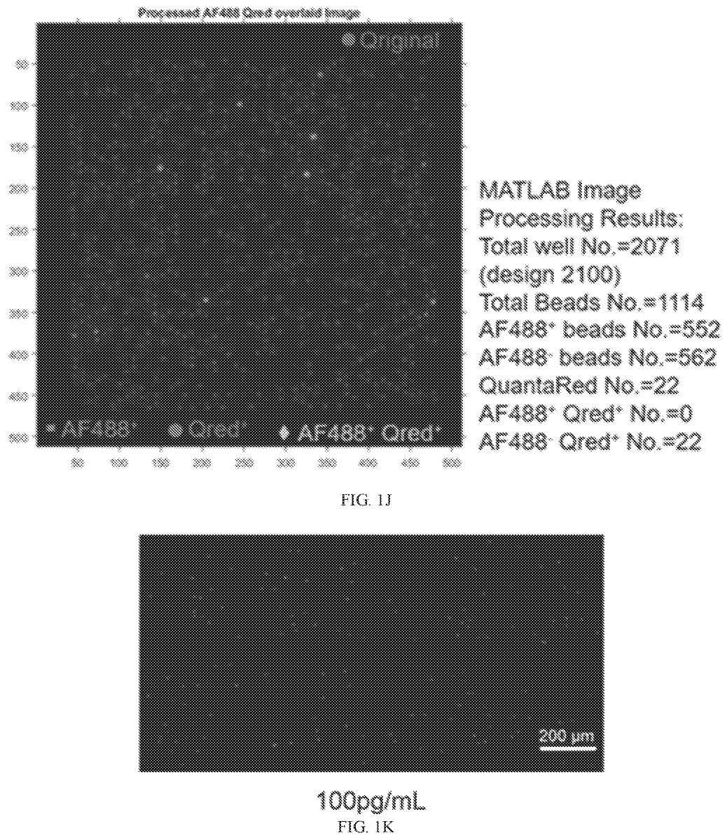

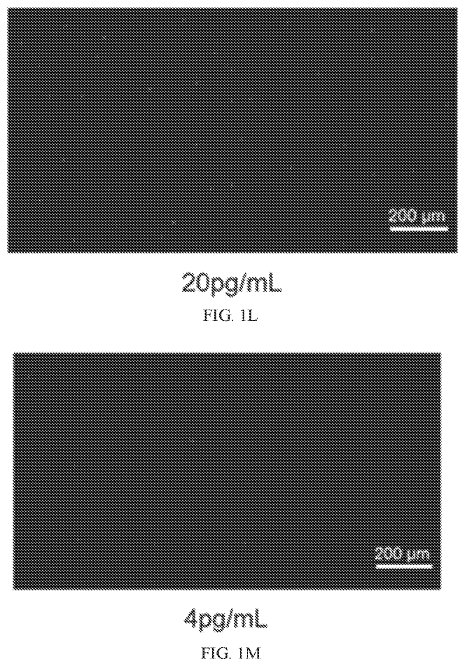

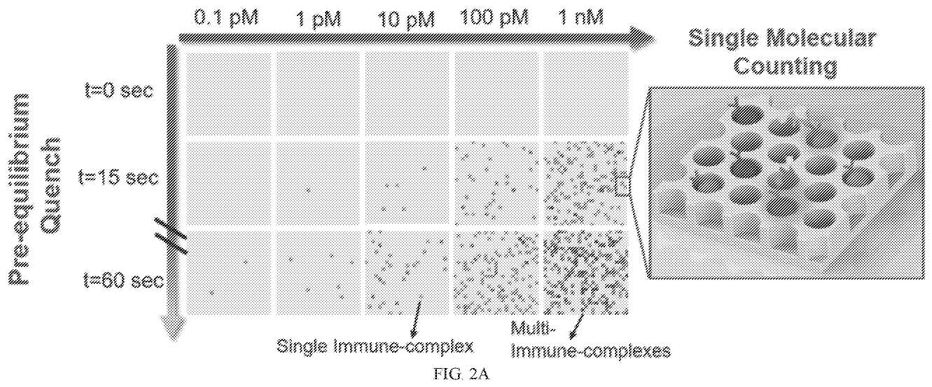

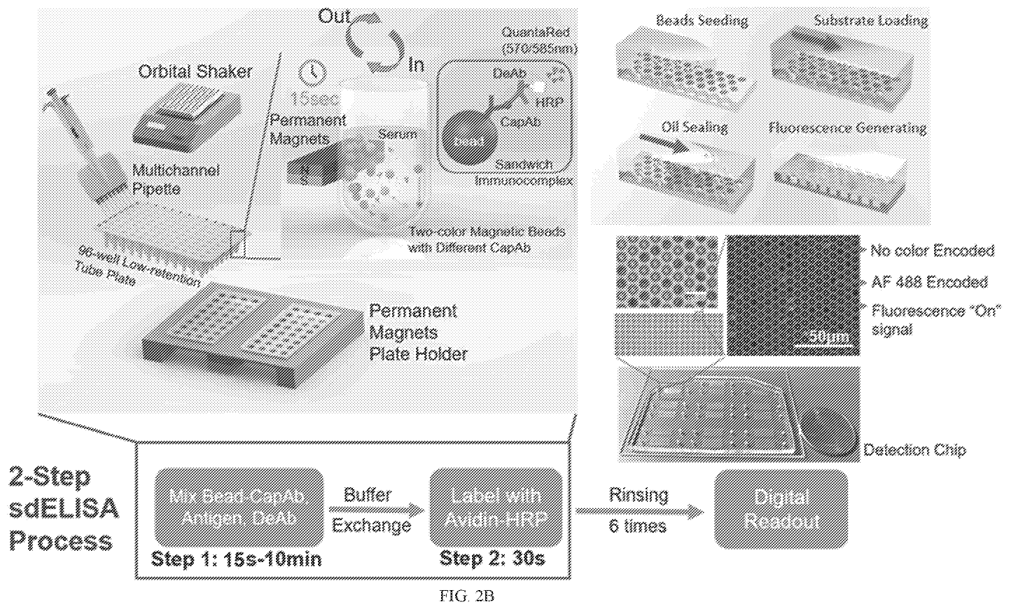

An exemplary overview of the PEdELISA concept of instantaneous single-molecule binary counting of preequilibrium protein binding events is shown (FIG. 2A). This example provides a combination of pre-equilibrium reaction quenching with single molecular counting to achieve an assay with a near-zero incubation time without losing linearity.

The PEdELISA process in this example employs a 2-step semi-homogeneous format so that it only involves (1) mixing the capture antibody-coated magnetic beads with the analyte and detection antibody solution to form the capture antibody-antigen-detection antibody complex (Step 1) and (2) labeling with enzyme HRP (Step 2) (FIG. 2B). To ensure accurate single-molecule counting at a wide range (10 fM-1 nM), which is relevant to clinical diagnosis, it was desirable to keep the population of fully labeled immune-complex molecules less than one per bead despite the original abundance of analyte molecules in each sample partition. In PEdELISA, the single-molecule counting condition was achieved by intentionally stopping the immunologic reaction in its early non-equilibrium state. This approach shortened the incubation time down to 15 to 300 sec for Step 1 and to 30 sec for Step 2. For practical operation, a fluid manipulation system was designed with a 96-well low-retention tube plate, a plate holder with permanent neodymium magnets, and an orbital shaker. The system was able to reduce the total sample volume to 10 μL, to efficiently pull the magnetic beads to the sidewall of the plate holder for buffer exchange, and to actively mix reagents in each reactor tube. Fluorescence-color encoded beads that were conjugated with different capture antibodies were used to achieve multiplex measurement at high throughput (FIG. 1).

The reaction process was followed by a digital signal detection process (FIG. 2), in which a custom-designed microfluidic detection chip was used to isolate individual beads into sub-arrays of 336,000 fL-sized partition wells. HRP reaction with a fluorescent substrate was used to indicate which beads were bound to antigen complexes. Confining the HRP catalyzed fluorophores to the tiny fL-sized volumes significantly amplified the readout signal up to single-molecule sensitivity for the immune-complex formation detection. The wells with activated fluorescence were imaged by an inexpensive full-frame CMOS camera and counted by a customized MATLAB code (FIG. 1 and FIG. 3). Color encoding of the beads identified the analyte type detected for each well. The average cost for reagents and chip fabrication was estimated at $0.69 per test (Table 1). The 2-step assay format incorporated the conventional enzyme labeling strategy using biotin-avidin linkages, which makes PEdELISA compatible with any commercially available reagents. The PEdELISA assay expenses are anticipated to be significantly reduced by scale-up production of the system. This would realize competitive cost advantages of PEdELISA over the current commercial ELISA ($2-5/test) or Luminex technologies ($30/test).

TABLE 1
Cost estimation of the PEdELISA assay

| | | | | No. | Cost/ |
|---|---|---|---|---|---|
| Disposable | Price | Total | Amount/ | of | test |
| and Reagents | ($) | amount | test | tests | ($) |
| Dynabeads ® | 534 | 60 mg | 6 μg | 10⁴ | 0.05 |
| Antibody | | | ~4 × 10⁵ | | |
| Coupling Kit | | | beads) | | |
| (ThermoFisher | | | | | |
| 14311D) | | | | | |
| Dynabeads ® M-270 | 415 | 60 mg | 6 μg | 10⁴ | 0.05 |
| Carboxylic Acid | | | (~4 × 10 | | |
| (ThermoFisher | | | beads) | | |
| 14305D) | | | | | |
| Purified anti-human | 250 | 500 μ | 36 ng | 1.39 × 10⁴ | 0.018 |
| IL-6 Antibody | | | | | |
| (BioLegend | | | | | |
| 459501110) | | | | | |
| IL-6 (Or MCP-1, | 459 | 10 × 96 | — | Min. | 0.037 |
| IL-2, TNF-α) Human | | ELISA | | 1.25 × 10⁴ | |
| Uncoated ELISA Kit | | test | | | |
| (ThermoFisher 88- | | | | | |
| 7066-88) | | | | | |
| Detection Antibody | Kit | 500 μL | 0.04 μL | 1.25 × 10⁴ | — |
| | included | (250×) | | | |
| Avidin HRP | Kit | 1.25 mL | 0.03 μL | 3.75 × 10⁴ | — |
| | included | (100×) | | | |
| Recombinant | Kit | 10 vials | — | — | — |
| protein standard | included | | | | |
| Blocking buffer | Kit | 150 mL | — | — | — |
| (1% BSA) | included | (5×) | | | |
| 96-Well Non-skirted | 39.55 | 25 × 96 | 3 | 800 | 0.05 |
| PCR Plates | | test | | | |
| (FisherScientific | | | | | |
| 14-230-232) | | | | | |
| Plain Glass | 63.26 | 1 gross | 16 | 2304 | 0.027 |
| Microslides | | (144 | tests/ | | |
| (FisherScientific | | slides) | slide | | |
| 12-550C) | | | | | |
| Dow Corning | 50.36 | 500 g | 1 g | 500 | .1 |
| Sylgard | | | | | |
| 184 Silicone | | | | | |
| Encapsulant | | | | | |
| Clear 0.5 kg Kit | | | | | |
| StartingBlock ™ | 188 | 1 L | 100 μL | 10⁴ | 0.019 |
| (TBS) | | | | | |
| Blocking Buffer | | | | | |
| (ThermoFisher | | | | | |
| 37579) | | | | | |
| QuantaRed ™ | 350 | 110 mL | 10 μL | 104 | 0.035 |
| Enhanced | | | | | |
| Chemifluorescent | | | | | |
| HRP Substrate Kit | | | | | |
| (ThermoFisher | | | | | |
| 15159) | | | | | |
| Others (pipette tips, | | | | — | 0.3 |
| microcentrifuge | | | | | |
| tubes, | | | | | |
| HFE Oil, washing | | | | | |
| buffers, Tween 20, | | | | | |
| cover slips, etc.) | | | | | |
| | | | | Total | 0.69 |

### Example 2

### Snapshot Single-Molecule Counting

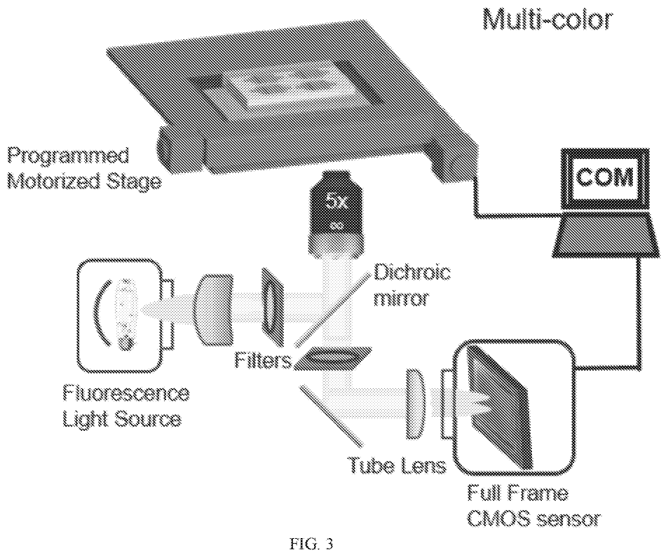

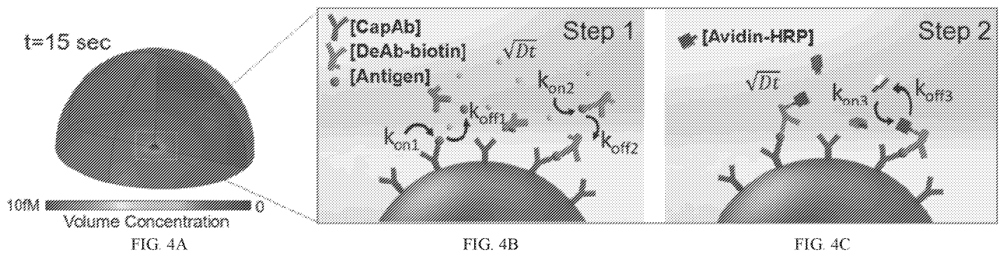

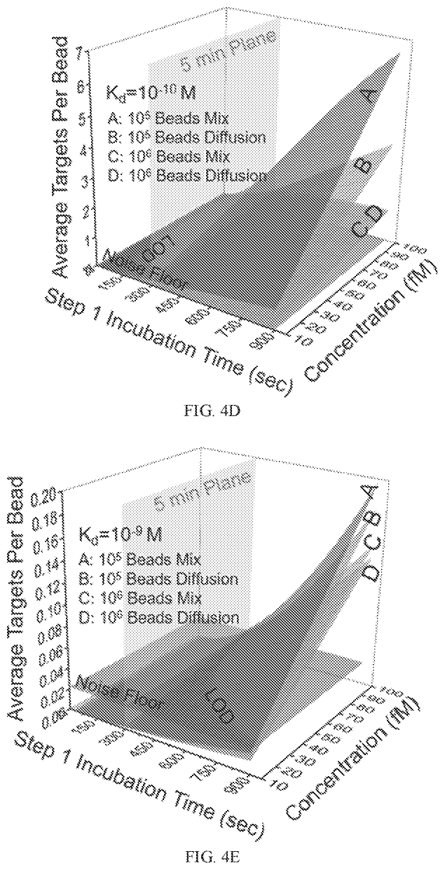

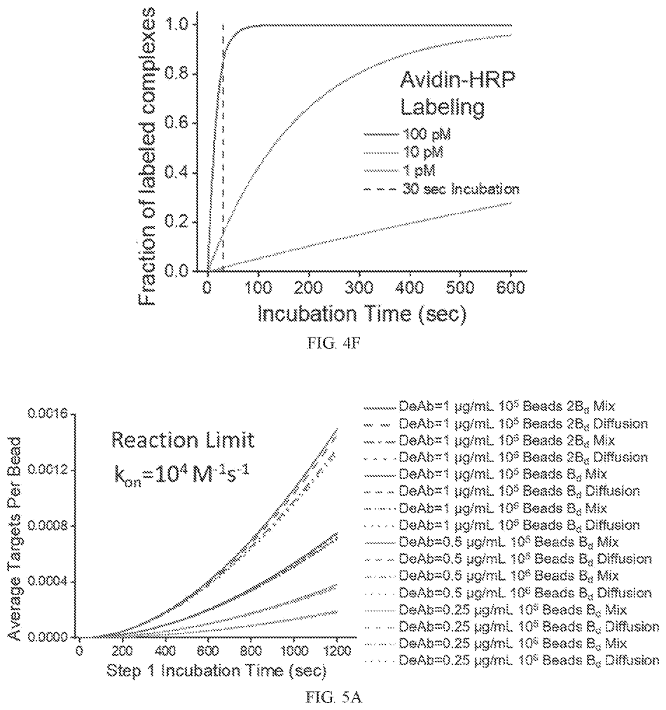

To validate the “quench-and-snapshot” approach, a finite element analysis (FEA) was carried out on biomolecular interactions in digital immunoassay and then a parametric analysis was performed to optimize the assay conditions. The analysis accounted for mass transport and surface reaction for a theoretical “reaction volume” with a bead placed in its center (FIG. 4A). Assuming that the beads were evenly distributed in the buffer/sample solution, the quantity of the reaction volume was taken to be the total buffer/sample volume divided by the number of the beads used for the assay. The simultaneous molecular interactions between the analyte molecules, the capture antibodies immobilized on the bead surface, and the detection antibodies freely floating in the reaction volume were modeled using the mass transport and Langmuir adsorption equations (FIG. 4B). For simplicity, the same affinity for the capture and detection antibody molecules was assumed. Then, the enzymatic labeling process was modeled for a biotin-avidin linkage with an affinity(34) of kₒₙ=5.5×10 M⁻¹s⁻¹ and k_{off}=3.1×10⁻⁵ s⁻¹ (FIG. 4C).

Using the key model parameters listed in Table 2, the kinetics of the antibody-antigen-antibody immune-complex formation process in Step 1 of PEdELISA were predicted for the affinity value (K_{d}=10⁻¹⁰-10⁻⁹ M) of typical commercially available antibodies. The average number of immune-complexes formed on a single bead surface, λ, were plotted as a function of the incubation time for the immune-complex formation process (Step 1 incubation time) and the analyte concentration (FIGS. 4D and 4E). The model shows that the kinetics of the immune-complex formation on the bead surface is non-linear with time due to the simultaneous interactions of the target analyte to both the capture and detection antibody molecules. Nonetheless, the model predicted a linear increase in the quantity of the formed immune-complexes with the analyte concentration independent of time, the analyte mass transfer type (forced advection or passive diffusion), and the number of beads. This linear relationship allowed the digital readout of PEdELISA to increase linearly with the analyte concentration even for a very short assay incubation time. This finding provided the theoretical foundation for securing both high sensitivity and a large linear dynamic range in this pre-equilibrium quenching approach.

TABLE 2
Key parameters for theoretical study

| | |
|---|---|
| Antigen Concentration | 10 fM~10 pM |
| Detection Antibody Concentration | 0.25-1 μg/mL |
| K_{on1&2} | 10⁴~10⁷M⁻¹s⁻¹ |
| k_{off1&2} | 10⁻³~10⁻⁵ s⁻¹ |
| Time Step 1 | 0-900 s |
| Bead Number | 10⁵-10⁶ |
| Sample Volume | 10 μ |
| Antibody/Bead | (1.8~3.6) × 10⁵ |
| Time Step 2 | 0~600 s |
| Avidin-HRP Concentration | 1-100 pM |
| kₒₙ₃ | 5.5 × 10⁸M⁻¹s⁻¹ |
| K_{off3} | 3.1 × 10⁻⁵s⁻¹ |
| Diffusivity | 10 μm²/s |
| Bead radius | 1.4 μm |

To optimize the assay conditions, the influence of several other crucial factors, such as the total number of beads per assay, the detection antibody concentrations, and the effect of sample-reagent mixing to enhance reagent mass transport was evaluated. For a system with a large antibody affinity value (K_{d}=10⁻¹⁰ M, FIG. 4D), the total number of beads and the mass transport both played crucial roles in determining the assay kinetics. In contrast, the Step 1 incubation time was the only dominant factor in determining the assay speed for a system with a weak affinity value (K_{d}=10⁻⁹ M. FIG. 4E). The PEdELISA assay uniquely provided the means to shorten the assay time for a reaction-rate limited weak-affinity system, in which other existing ultrafast immunoassay methods primarily driven by mass transport enhancement through active mixing or surface-to-volume ratio enhancement failed to achieve. FIG. 4F shows the kinetics of the labeling process for three representative avidin-HRP concentrations and suggested that the concentration of 100 pM was sufficiently large to complete the process with the incubation time (Step 2 incubation time) of 30-sec.

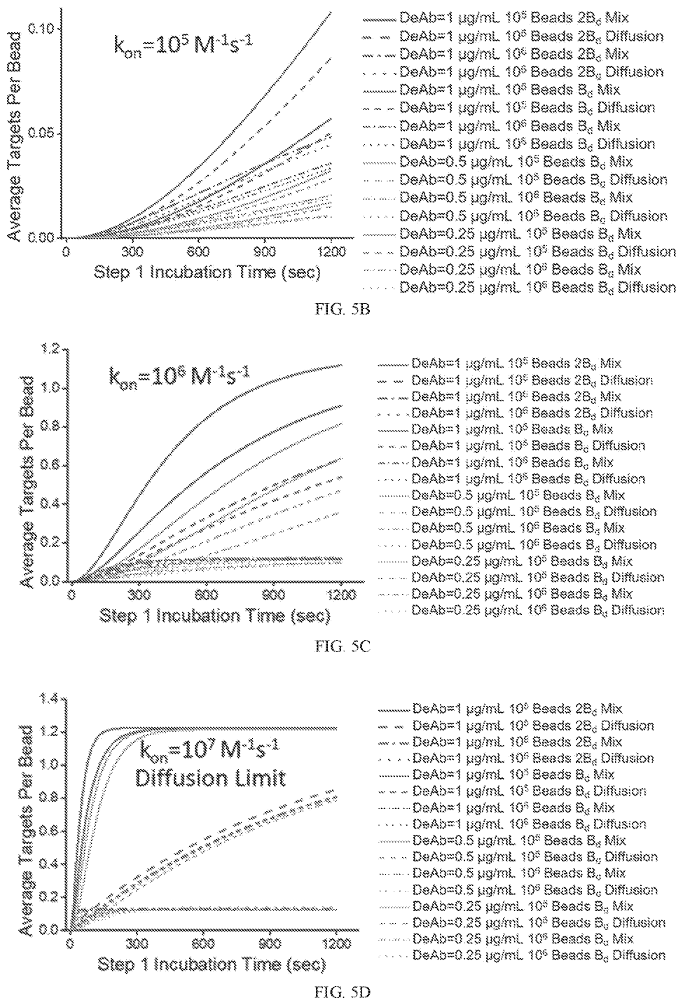

Parametric Analysis and Optimization of the Assay Outcome. The influence of several other key assay parameters on the binding kinetics in the Step 1 reaction (FIG. 5) was evaluated to obtain optimal assay performance. These parameters included the detection antibody (De_{Ab}) concentration, the number of magnetic beads used per sample, and the capture antibody binding site density (B_{d}). The parametric analysis was performed for different mass transport conditions with and without active mixing (diffusion only) with different on-rate association constant (kₒₙ) values ranging from 10⁴ to 10⁷ M⁻¹s⁻¹, which represent different affinities of the antibody-antigen pair. The detection antibody concentration was limited to <1 μg/mL in the calculation. This allowed the assay to avoid experiencing high background non-specific adsorption in practice. The magnetic bead amount was kept above 10⁵ per reactor (e.g., PCR tube) to ensure statistically significant digital counting. The number of the capture antibody conjugated per magnetic bead was estimated based on the experimental data provided by Dynabeads® Antibody Coupling Kit Manual (Catalog number: 14311D).

For a case where the antibodies have a very weak affinity (kₒₙ=10⁴, FIG. 5A), the immune complex formation kinetics was limited by the rate of surface reaction between the antigen and capture antibody molecules. The calculation indicated that neither mixing-driven mass transport enhancement nor the number of beads (equivalent to sample volume) would improve the assay performance in this “reaction-limited” regime. Although increasing the binding-site density (B_{d}) or detection antibody concentration helped increase the assay signal, it would be impractical or costly to largely increase these two parameters. The average number of capture antibody conjugated per bead was almost invariant on the bead surface area. Meanwhile, blindly increasing the detection antibody concentration would generate a high level of background false positives due to non-specific surface adsorption.

For another extreme case where the antibodies have a very strong affinity to the analyte (kₒₙ=10⁷, FIG. 5D), the immune complex formation kinetics was limited by the mass diffusion of the originally unbound antigen and detection antibody molecules. Compared to the incubation time, the mass transport condition, the number of beads, and the reaction volume size were more dominant factors affecting the assay readout in the “diffusion-limited” regime. For example, with a large number of beads (10⁶) in a small sample volume (20 μL), the reaction was shown to quickly reach the equilibrium state within 2 min due to the fast reaction rate and the small number of target molecules per bead. This was exactly the case that was wished to be avoided in the digital immunoassay. In the “diffusion-limited” regime, a depletion region growing near the bead surface over time was the primary factor limiting the transport of target molecules to the capture sites from the far-field. Enhancing mass transport of these molecules through active mixing would help reduce the depletion region growth.

Commercially available antibodies for cytokine detection generally have affinity values falling between those in the above two extreme cases. Based on this study, it was determined that a strategy of preparing beads densely coated with capture antibody molecules, using a small number of these beads in a relatively large sample volume, and enhancing mass transport by active mixing would help achieve ultrafast PEdELISA assay without sacrificing its sensitivity.

### Example 3

### Analytical Validation and Benchmarking of the Assay

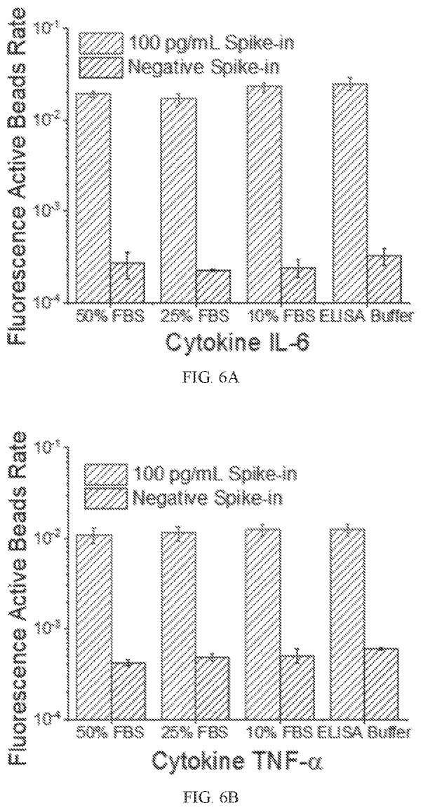

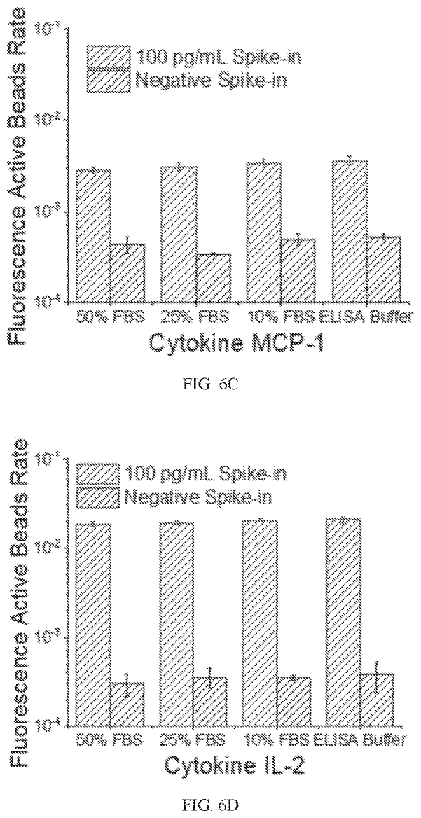

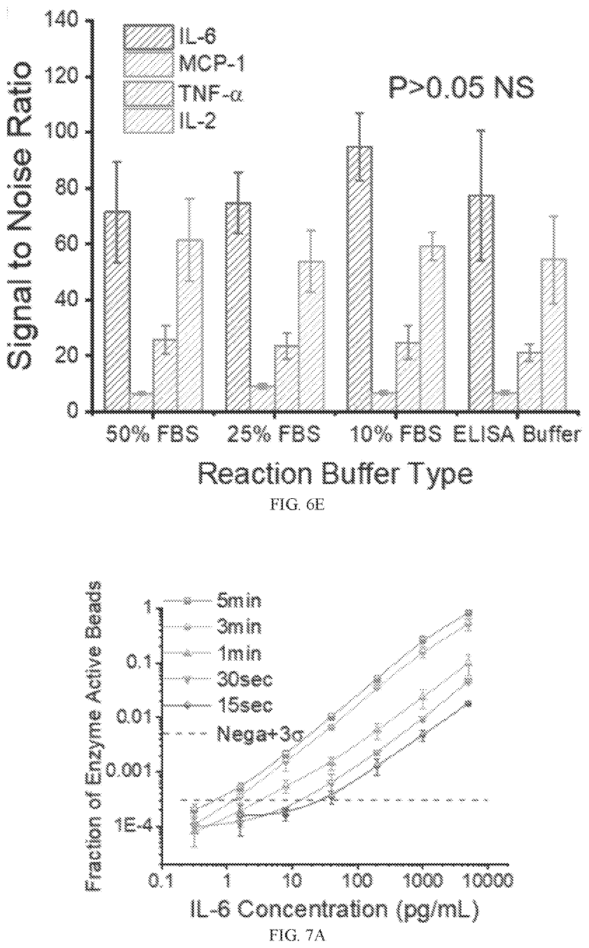

To experimentally characterize the PEdELISA assay performance, four signaling cytokine biomarkers involved in the progression of cytokine release syndrome (CRS), a significant complication of CAR-T that impacts morbidity and mortality were selected: IL-6; TNF-α; IL-2; and MCP-1. To examine the impact of the different levels of background protein on the digital immunoassay signal pertaining to these fluids, four different types of buffers were spiked with 100 pg/mL each cytokine: the 1×ELISA diluent (1% BSA, 0.05% Tween20), 10%, 25% and 50% fetal bovine serum (FBS) (FIGS. 6A-6D). The signal-to-noise ratio (SNR), which is defined as the measured signal divided by the average blank signal+3σ, was calculated. In general, a larger surface blocking effect, perhaps, owing to the presence of albumin, was observed for serum media, which resulted in a slightly lower spike-in signal and background noise compared to the ELISA buffer. However, there was no significant difference in the SNR value between the different media groups (P>0.1 n=5-8, one-way ANOVA. FIG. 6E).

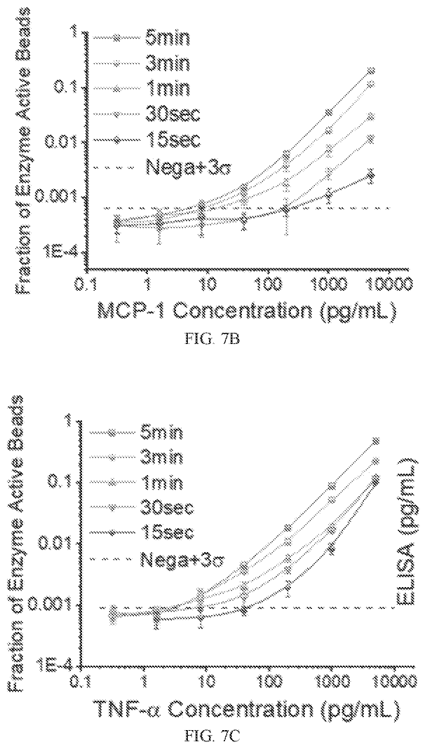

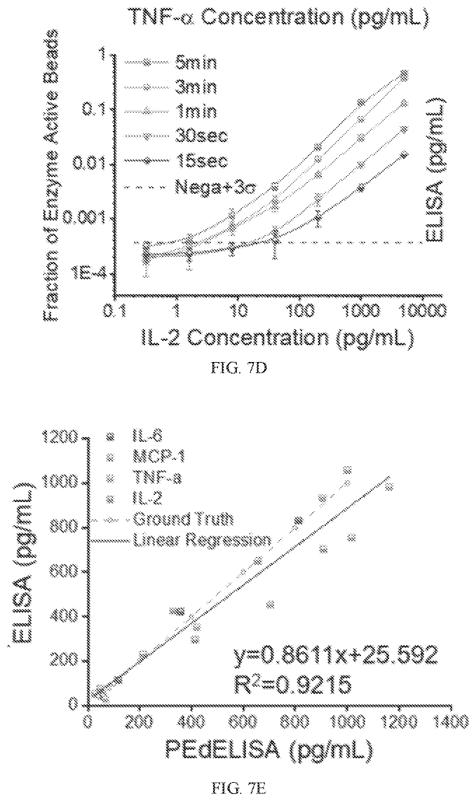

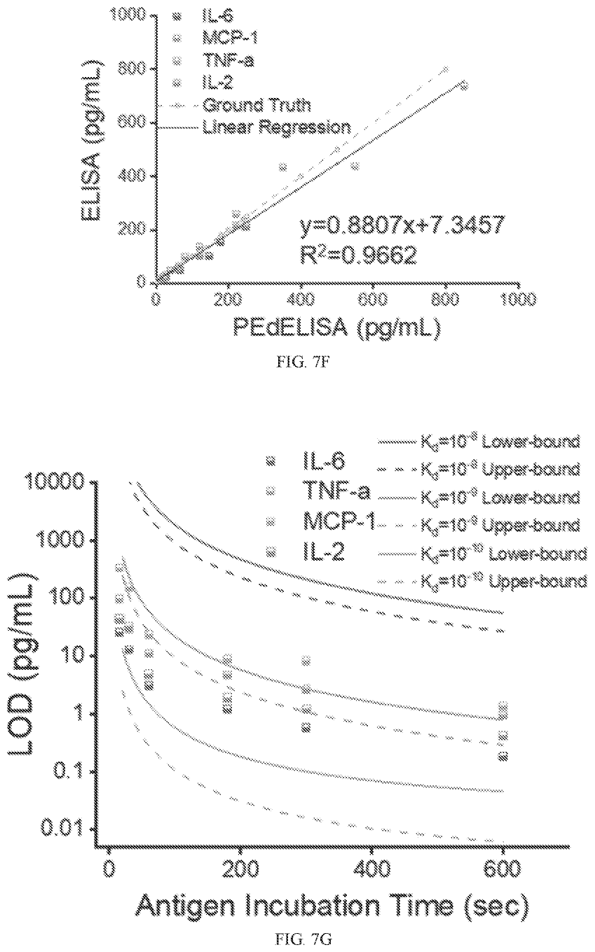

FIG. 7A-7D show standard curves for the four cytokines ranging from 0.32 pg/mL to 5 ng/mL in 25% fetal bovine serum (FBS) with the Step 1 incubation time varying from 15 to 300 sec while fixing the Step 2 incubation time at 30 sec. To push the limit of measurement speed, the assay was tested and successful with 15-sec (Step 1) and 30-sec (Step 2) incubation times by simply mixing all three reagents (detection antibody, avidin-HRP, antigen) with magnetic beads (1-step assay format). As theoretically predicted, the digital readout (the fraction of fluorescence-activated “On” beads to the entire beads) was highly time-dependent and in general, linearly proportional to the analyte concentration. A variation in the signal output was observed depending on the cytokine species. This was likely due to the difference in the antibody pair affinity across the different cytokines.

Notably, the linearity of the assay was confirmed over a three-order-of-magnitude concentration range regardless of the analyte type and was quite well maintained even for the 15-sec ultrafast PEdELISA assay of IL-6 (the primary mediator in CRS). Thus, quenching the extremely preequilibrated reaction did not compromise the measurement resolution, which indicates PEdELISA would be suitable for practical clinical diagnosis.

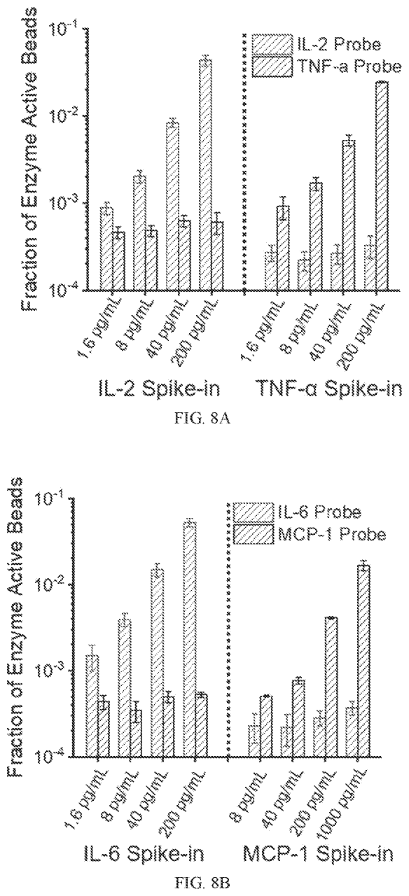

The assay was further validated by comparing measurement results for spiked-in FBS samples between the conventional 3-step sandwich ELISA and PEdELISA with the Step 1 incubation time of 15-sec (FIG. 7E) and 300 sec (FIG. 7F). The ground truth (spike-in) value of the analyte concentration was set between 40 pg/mL (2 pg/mL) to 1000 pg/mL for the 15-sec (300-sec) assay. Good agreement was found between the two methods for both the 15-sec (R²=0.92) and 300-sec (R²=0.96) assays. Considering the noise floor of the experimental setup, the minimum incubation time for a given target value of LOD using the model was theoretically predicted and compared with the experimental data (FIG. 7G). An excellent match was observed between the experimental data and the theoretical curves with the assumption of typical antibody affinities ranging from 10⁻⁹-10⁻¹⁰ M. Additionally, the dual-plex assay's specificity was verified by quantifying the different antibody pair's cross-reactivity (FIG. 8). The assay LODs and the root mean square coefficient of variance (RMS CV %) accumulated over the titration experiments for the four cytokines are summarized in Table 3.

TABLE 3
Limit of detection (LOD) and standard root mean square
coefficient of variance (RMS CV) of 15-sec and
5-min PEdELISA. LOD was determined by
concentration from tbe reagent blank’s signal + 3σ.

| | Patient | Assay | LOD | LOD | Patient Assay |
|---|---|---|---|---|---|
| Cytokine | Sample | Blank + | 15-sec | 5-min | standard RMC |
| Type | Volume (μL) | 3σ (%) | (pg/mL) | (pg/mL) | CV (%) |
| IL-6 | 5 | 0.03034 | 25.9 | 0.58 | 17.3 |
| MCP-1 | 5 | 0.06402 | 333.2 | 8.29 | 27.5 |
| TNF-α | 10 | 0.09152 | 96.7 | 2.61 | 24.4 |
| IL-2 | 10 | 0.03671 | 43.9 | 1.22 | 17.8 |

### Example 4

### Longitudinal Biomarker Profile Measurement for Septic Mice

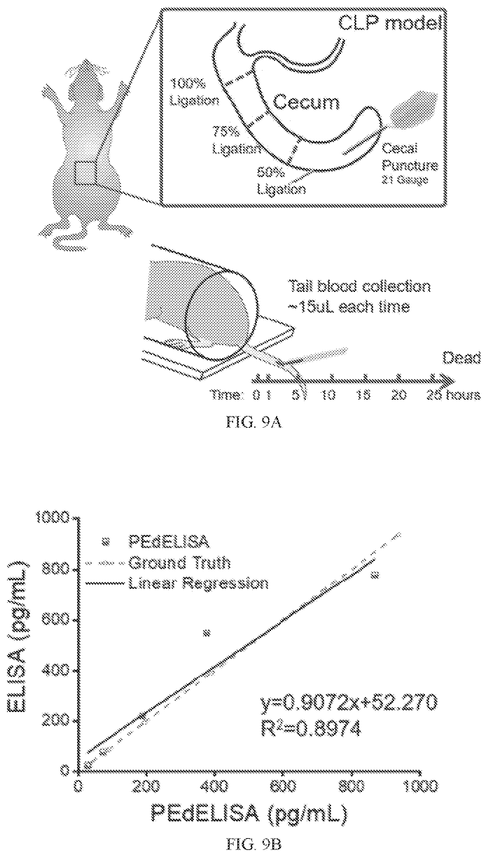

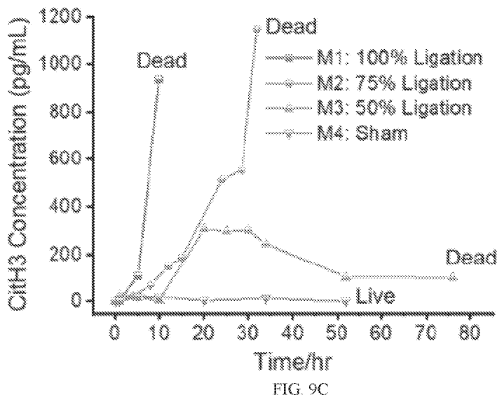

To demonstrate the sample-sparing capability (5 μL), the PEdELISA assay was used in a mouse model study. Continuous monitoring of a biomarker profile in a living mouse is impossible with the existing ELISA assay as it requires ˜0.5 mL of whole blood (for duplicate assay) for each time points, which exceeds the amount available from a single mouse. As a result, the conventional technique requires sacrificing the mouse at each measurement to collect a sufficient quantity of blood. For this test, mouse models of CLP-induced septic shock (more clinically realistic model reflecting polymicrobial infection) were prepared with their cecum ligated at 50, 75, and 100% of the total length (FIG. 9A). Then, a version of the PEdELISA assay (FIG. 9B) for quantifying the biomarker citrullinated histone H3 (CitH3) was developed and validated. Catalyzed by peptidylarginine deiminase (PAD), CitH3 has recently been identified as an early step in neutrophil extracellular traps (NETs)-induced immune cell death (NETosis) in response to infection. The dynamic variations of CitH3 were evaluated in four mice with different CLP-induced septic shock severity over their lifetimes (FIG. 9C). Blood samples were collected through the tail vein every 4-5 hours from 0, 1, 5, 10 hr until the animal died. To ensure the tail-cutting would not affect the disease progression, only 15 μL of the whole blood was collected through a capillary tube at each time point, and a sham mouse without CLP was prepared following the same surgical procedure. Blood samples were processed to obtain 5-7 μL serum and were then subsequently quantified by the PEdELISA.

For the 100% CLP mouse, a significant increase of CitH3 was observed at 5 hr time point (106.6 pg/mL) and the mouse was found dead within 12 hr. For the 75% CLP mouse, the increase of CitH3 was relatively delayed comparing to the 100% CLP mouse. But CitH3 continued to increase and reached a peak value of 1149.2 pg/mL at around 32 hr when the 75% CLP mouse died. For the 50% CLP mouse, no significant increase of CitH3 was observed in the first 10 hr, but then CitH3 started to increase between 10 and 20 hr and reached its plateau (˜300 pg/mL) at 20-30 hr. The physical condition of the 50% CLP mouse recovered at 24-48 hr, and the CitH3 level went down during that period. However, the condition of the 50% CLP mouse quickly became worse after 70 hr and the mouse was found dead at 76 h. For the sham case, the CitH3 level stayed low <21.5 pg/mL and the mouse had stayed alive during the entire experiment.

### Example 5

### Near-Real-Time Multiplexed Cytokine Monitoring of Post-CAR-T Cell Infusion CRS

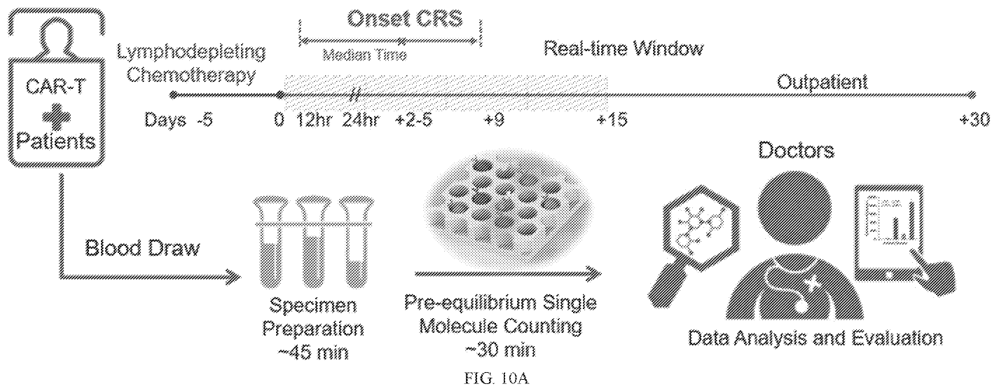

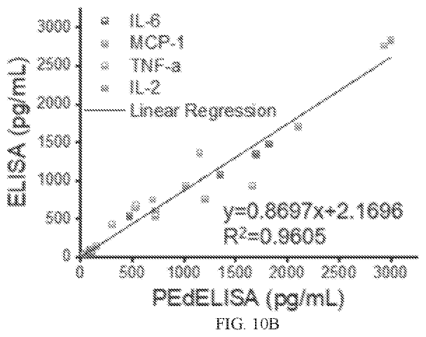

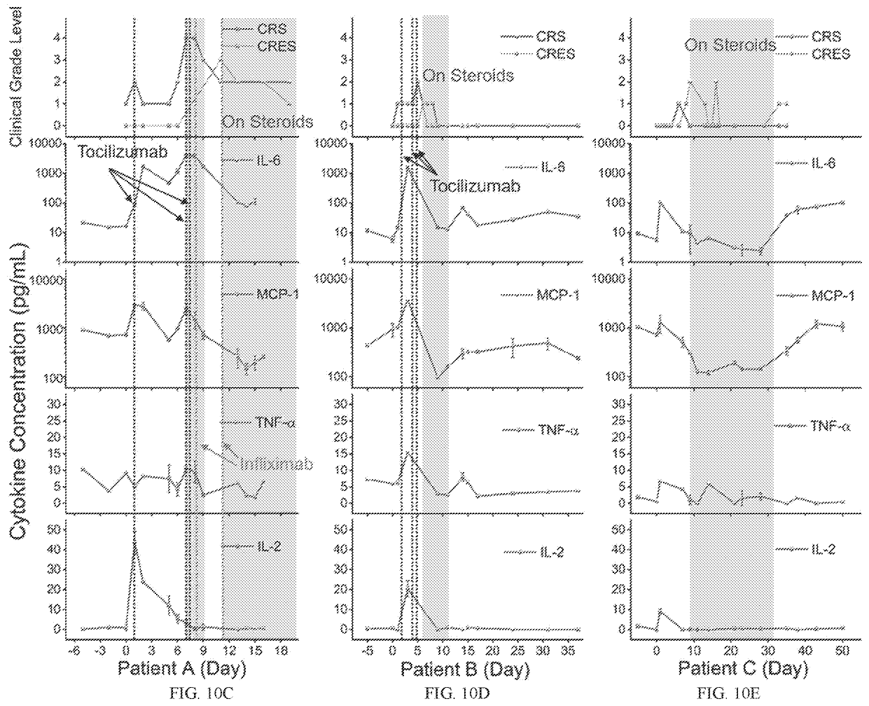

The PEdELISA was applied to real-time monitoring of the IL-6, TNF-α, IL-2, and MCP-1 profiles of hematological cancer patients showing severe (Patient A), moderate (Patient B), and mild (Patient C) CRS symptoms after CAR-T cell therapy following a pre-approved sample collection protocol. CRS is a form of systemic inflammatory response accompanied by a high level of inflammatory cytokines released into the bloodstream by activated white blood cells. It can rapidly evolve (i.e., within 24-48 hours) from manageable constitutional symptoms (grade 1) to more severe forms (grade 2-4), for which rapid and sensitive serum cytokine measurements could direct urgent interventions. Here, PEdELISA allowed real-time cytokine profile measurements to be performed for blood samples drawn in ICU according to the timeline shown in FIG. 10A. In general, daily blood draw started five days before the infusion for baseline collection until the patient was discharged. For the real-time monitoring, the sample was first processed within 45 min of blood draw to extract serum and then tested by the PEdELISA within an hour. The data typically became available for clinicians within 2-3 hours from the initial point of patient blood collection. The initial onset of CRS and the development of neurotoxicity of three patients were labeled along the timeline.

To ensure the highest accuracy and sensitivity for these clinical measurements, the total incubation time was 300-sec (Step 1)+30-sec (Step 2) in the 2-step assay format. Banked CAR-T patient serum samples (n=23) with unknown analyte concentrations were first assayed by 2-step PEdELISA and validated by ELISA (FIG. 10B). The data between these two methods showed excellent linear correlation (R2=0.96), which confirmed the accuracy of the 2-step PEdELISA assay for human biomarker detection. Then, real-time measurements were performed which captured the patients' complex immune responses to the immunomodulatory interventions manifested by the time-course variations of the cytokine profiles (FIGS. 10C-10E). The dynamic behavior of these responses varied from patient to patient and decisions for treatment of CRS in these patients were made solely based on clinical criteria (e.g., CRS grades) which did not involve any serum cytokine data.

For Patient A, who had a high tumor burden, the time to initial onset of CRS was as short as 13.5 hours. The MCP-1 and IL-2 levels rose rapidly and reached the peak values (MCP-1 2947 pg/mL; IL-2 39.72 pg/mL) within 24 hours after CAR-T infusion, which correlated with the patient's grade 2 CRS accompanying the fever (39.3° C.) on Day 1 (FIG. 10C). The continuous rise of IL-6 was also observed transiently after the first tocilizumab administration (Day 1) from 89.9 pg/mL (Day 1) to its peak level of 1676 pg/mL through Day 2. A similar trend was noted for Patient B (FIG. 10D), who received the first dose on Day 2 with grade 1 CRS, and the peak (1546 pg/mL) was detected on Day 3. Patient A later developed grade 3-4 life-threatening CRS on Day 6-9 and was readmitted into the ICU. During this period, the IL-6 level approached an extremely high level of 4383 pg/mL (Day 7) and 4189 pg/mL (Day 8), and MCP-1 reached its second peak, 2103 pg/mL, despite the fact that Patient A was administered with multiple immunosuppressing agents. Interestingly, a significant rise of TNF-α and IL-2 was not observed during the second CRS peak. The patient was diagnosed with grade 3 CRES (CAR-T cell-related encephalopathy syndrome) on Day 11. For Patient B (FIG. 10D), one peak for all four cytokines was observed during the first eight days after CAR-T cell infusion, which correlated with the patient's clinical symptoms during this period (Grade 1-2 CRS). Patient C did not develop CRS until 6 days after the CAR-T cell infusion, although a slight elevation for all four cytokines was observed on Day 1 (FIG. 10E). However, Patient C developed prolonged neurotoxicity starting from Day 8 and was on steroids from Day 9 to Day 33. During this period, all four cytokines stayed at low levels. After steroids were stopped, Patient C's IL-6 and MCP-1 levels rose back from Day 35 to Day 50 and grade 1 neurotoxicity relapsed.

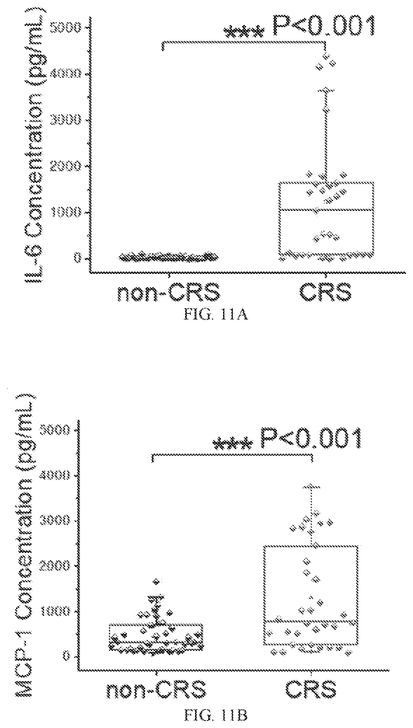

The time-series cytokine data in FIG. 10 was sorted and combined for the three CAR-T patients into non-CRS and CRS (grade 1 or higher) groups (FIG. 11). Both IL-6 and MCP-1 strongly correlated with CRS (P<0.001). The data also indicated a similar correlation between IL-2 and CRS with a lower statistical significance (P=0.0059). However, a significant statistical difference was not observed for TNF-α between the non-CRS and CRS groups (P=0.142). The data showed a consistently low level of TNF-α for both groups across all the three patients.

### Example 6

### Highly Multiplexed Pre-Equilibrium Quenching Digital ELISA (PEdELISA) Microarray Processed by Convolutional Neural Network

The existing dELISA multiplexing method (4-6 plex) which utilizes only fluorescence dye encoded magnetic beads requires a significant number of beads and large sample volume, is plagued by serious optical cross-talk which can sacrifice sensitivity and accuracy. In addition, there is lack of a highly accurate and reliable signal analysis algorithms that can process multi-color, digital counting of millions of micro-reactors in a few minutes. The Convolutional Neural Network (CNN)-processed PEdELISA platform described below addressed these challenges by extending the multiplex capacity with near-real-time assay turnaround, which has great potential for time-sensitive disease diagnosis and assists critical care physicians to implement timely therapeutic interventions precisely guided by real-time biomarker profiles.

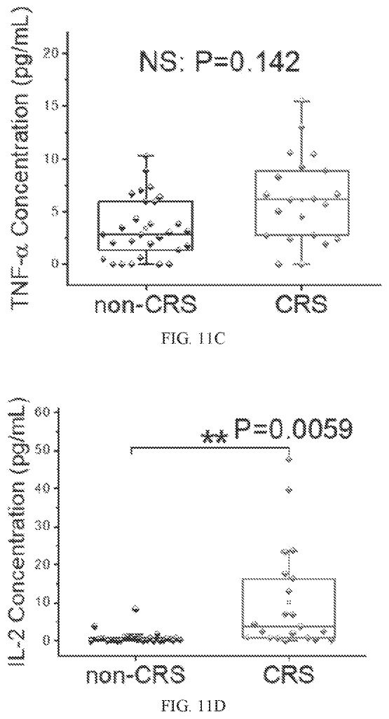

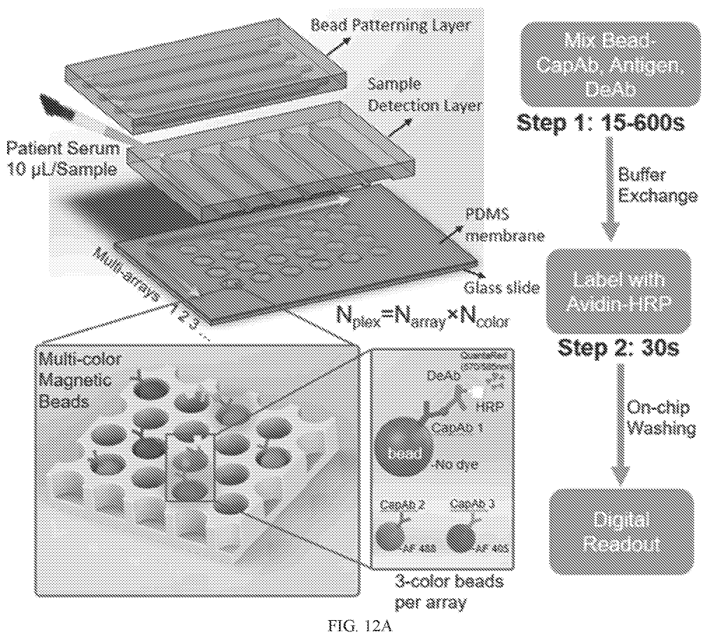

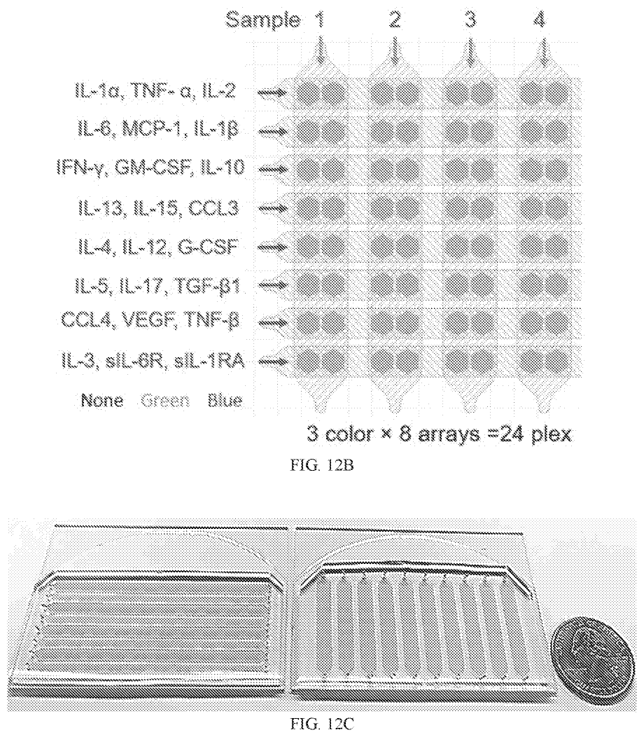

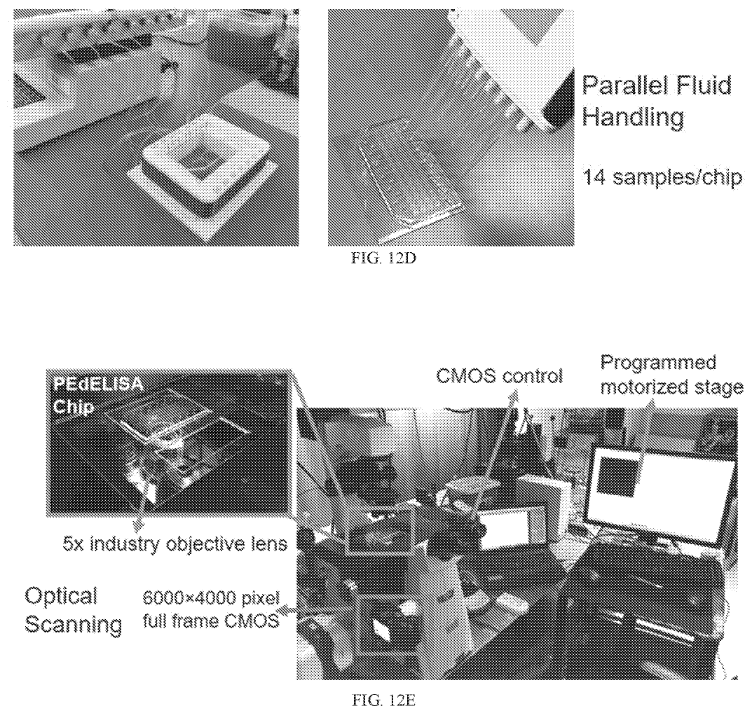

The PEdELISA was extended to a highly multiplexed (24-plex, and potentially more) microfluidic format as shown in FIG. 12. The PEdELISA chip contained three layers (FIG. 12A, from bottom to top): the glass slide substrate (1 mm, optical transparent), the thin PDMS membrane layer (200-500 μm) where microwell (d=3.8 μm) structures were fabricated on the surface (soft-lithography), the top chamber layer for either magnetic beads patterning or sample detection (3-5 mm, interchangeable). Using the bead patterning layer, multiple colors of magnetic beads were patterned (conjugated with different capture antibodies) into physically separated microarrays to create a high multiplex capacity equal to N_{color}×N_{array} (e.g., 3 colors×8 arrays=24 plex). The patterning layer was peeled off and attached to the sample detection layer (designed perpendicular to the patterning layer) for the protein biomarker detection. Biological samples (e.g., serum) were loaded through a multichannel pipette. In addition, single-molecule counting was applied to the PEdELISA system. A 24-plex CAR-T cytokine panel was designed as shown in FIG. 12B for the PEdELISA system as shown in FIGS. 12C-12E, which constitutes the PDMS-based microchip described above, a parallel fluidic handling unit (14 samples/chip), and a two-color fluorescence optical scanning unit.

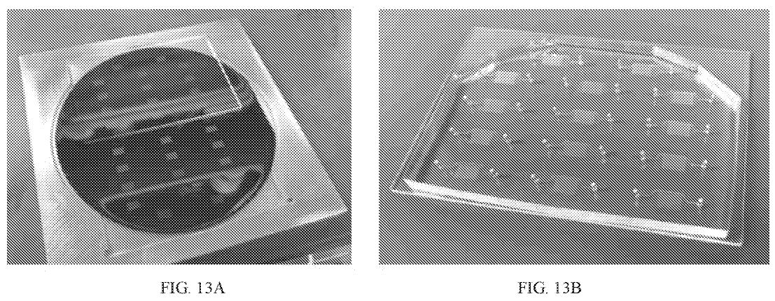

The PEdELISA platform is extremely low cost without sacrificing the performance in comparison with competing methods for clinical translation (Table 4). This low-cost feature mainly comes from the molding-based chip microfabrication. Conventional methods typically require cleanroom facilities to directly micro-fabricate the required features on an assay chip using photolithography and reactive ion etching, for example. Therefore, each individual chip can be very expensive, have a batch to batch difference and cannot be reused due to the concern of sample contamination. A jig-guided large PDMS thin film transfer technique was developed (FIG. 13) which resulted in the micro-fabricated feature being directly transferred onto a glass substrate (air plasma treated) without the access for cleanroom facility. A silicon mold was made and all the micro-features were repeatedly made using the mold by PDMS-molding and glass slide transferring. In addition, the microfluidic design allows extremely low-volume reagent consumption (<$5 for 24-plex cytokine assay). Cost-effective consumer-grade equipment was selected, such as SONY full-frame CMOS camera, and the system was designed in modules to further reduce the overall cost and allow compatibility with standard epifluorescence microscopy found in a typical clinical lab.

TABLE 4
Cost estimation of the PEdELISA assay versus
competing immunoassay diagnostic systems

| | Cost/ | In- | Total | | |
|---|---|---|---|---|---|
| Diagnostic | 96 | strument | Assay | LOD | |
| system | assays | Cost | Time | (pg/mL) | Plexity |
| Luminex | $3000 | >$40,000 | >4 hours | 0.1-100 | 25-65 |
| Colorimetric | $200 | $5,000 | >4 hours | 1-10 | 1 |
| ELISA | | | | | |
| Quanterix ™ | $1000 | >$200,000 | >45 min | 0.002-1 | 4-10 |
| SIMOA ® HD-1 | | | | | |
| PerkinElmer | $1200 | >$200,000 | 4 hours | 1-100 | 4 |
| AlphaLISA ® | | | | | |
| PEdELISA | $50 | <$10,000 | <30 min | 0.1-5 | 24 |
| Platform | | | | | |

*Total assay time include sample preparation, analyte quantification, data. acquisition, and data

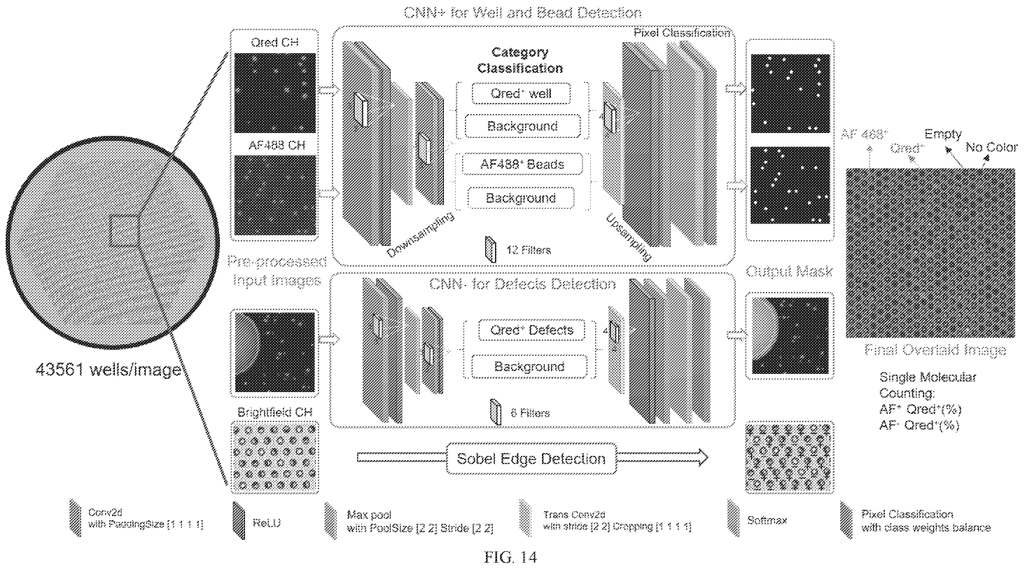

The PEdELISA signal was processed by a novel MATLAB-based bi-direction CNN algorithm which was pre-trained to recognize fluorescence “On” wells (Red channel. Qred) or beads (Green channel, AF488) versus defects and contaminations using 5750 labeled images (FIG. 14). In the current PEdELISA detection, three types of raw images were collected: 1. Red fluorescence channel (Qred) 2. Green fluorescence channel (AF488) 3. Brightfield channel. The first two channels were sent directly to the bi-direction CNN to classify the Qred⁺ microwells, AF488⁺ beads, image defects, and background. Then the Qred⁺ and AF488⁺ targets were segmented out as the output mask and the defects were removed. The bright-field image was analyzed using the Sobel edge detection methods to determine the overall beads filling rate. After post-image processing, the code overlayed the three images to determine the numbers of Qred “On” microwells with and without an AF488-dyed bead inside. The fractional population of the enzyme active Qred “On” microwells to the bead-filled microwells for both the AF488 dyed (AF⁺ Qred⁺ %) and non-color bead types (AF⁻ Qred⁺ %) is directly proportional to the different biomarker concentrations to be determined.

The architecture of the network consists of 10 layers, including three 2D convolution layers (4-6 filters, kernel of 3-3) with three rectified linear unit (ReLU) layers, a 2D max-pooling layer (stride of 2), a transposed convolution layer with ReLU, a softmax layer and a pixel classification layer. To speed up the training process, labeled 32×32 pixel images were used to pre-train the network and then used the pre-trained network was used to label the 256×256 images for the final network building. To mitigate the large intensity difference among each micro-reactor, the same scale was used to label either the bright and dim wells or beads. Since the majority of pixel labels are for the background in typical digital assay images, the inverse frequency weighting method where the class weights are the inverse of the class frequencies were used to mitigate the class imbalance issue and increase the weight given to under-represented classes. The bi-direction CNN network described herein significantly increased the PEdELISA signal processing accuracy comparing to conventional global thresholding image processing method as shown in FIG. 15. It took less than 10 sec to process a 6000-4000 image which contains 43561 micro-reactors using a standard 6′ gen i7 processor (or above).

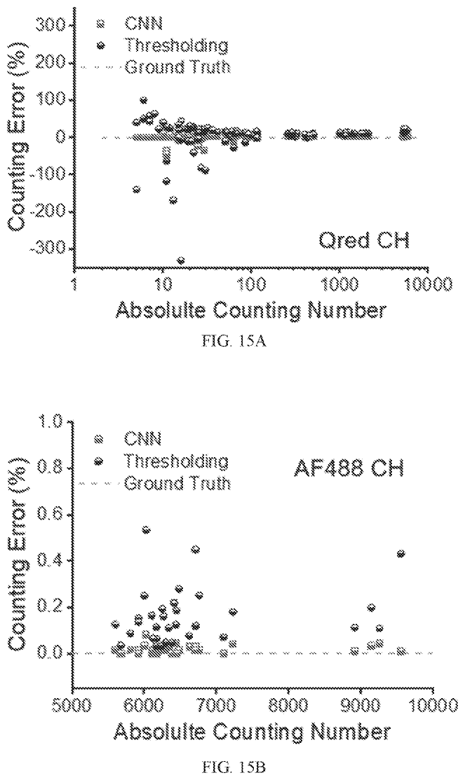

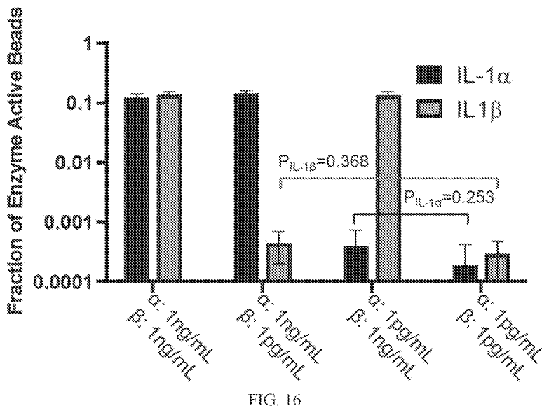

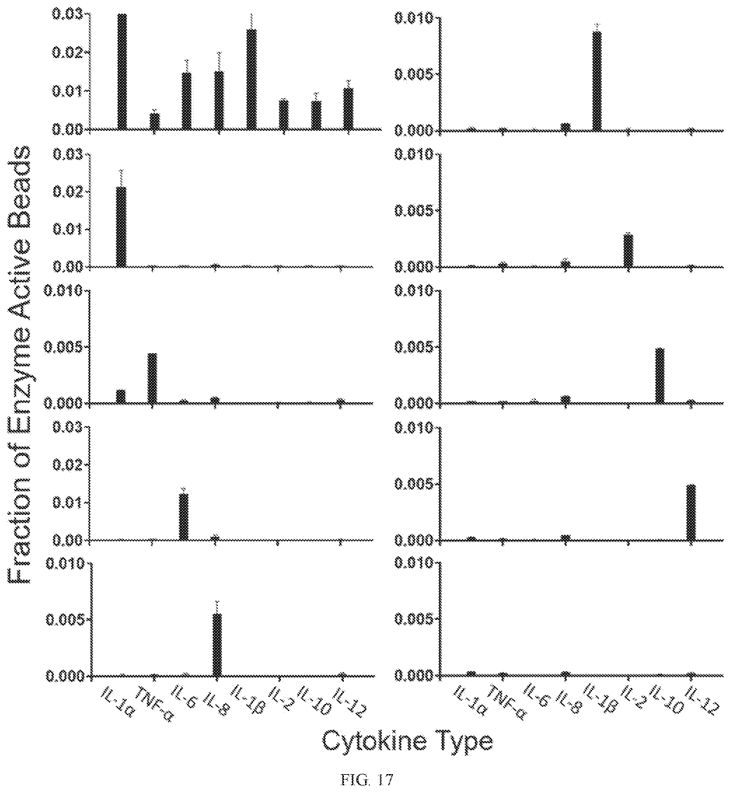

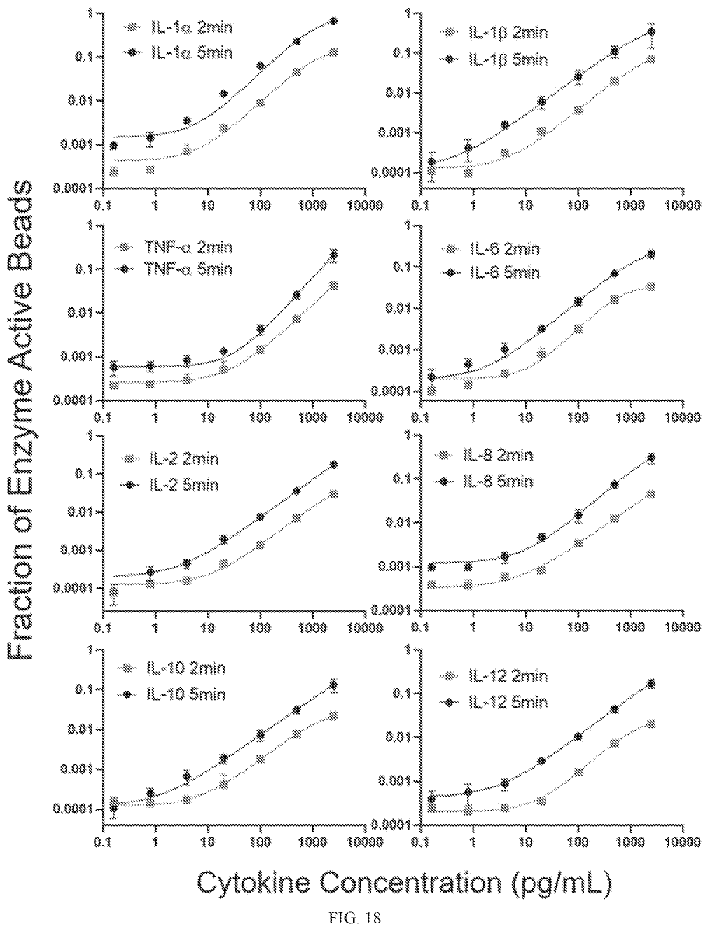

To characterize the PEdELISA performance, the potential for multiplex cross-talk errors was determined from the assay and signal processing side:optical cross-talk and antibody cross-reactivity. To check the optical cross-talk suppression capability of the developed CNN network, a serum sample spiked with two different cytokine species of 1000-fold concentration difference, IL-1α (AF488 encoded) and IL-1β (non-color encoded), were prepared. FIG. 16 shows the CNN-processed raw signal of 1 pg/mL IL-1α or β will not be interfered even when the other protein reaches 1 ng/mL (two-tailed unequal variance t-test, P>0.1). The specificity of the assay was checked by spiking 100 pg/mL of each individual cytokine in 25% fetal bovine serum (FBS) and no significant antibody cross-reactivity was observed among the developed 8-plex assay (FIG. 17). FIG. 18 shows standard curves obtained within a 5-min and 2-min assay incubation time for eight cytokines ranging from 0.16 pg/mL to 2.5 ng/mL in 25% FBS. The reaction conditions were optimized to match all cytokines within the clinically relevant dynamic range, and a linear dynamic range of nearly four orders of magnitude was achieved. The limit of detection for the 5-min assay time is below 1 pg/mL and for the 2-min assay is in between 1-10 pg/mL for all 8 cytokines.

### Example 7

### 14-Plex Pre-Equilibrium Quenching Digital ELISA (PEdELISA) for Cytokine Analysis

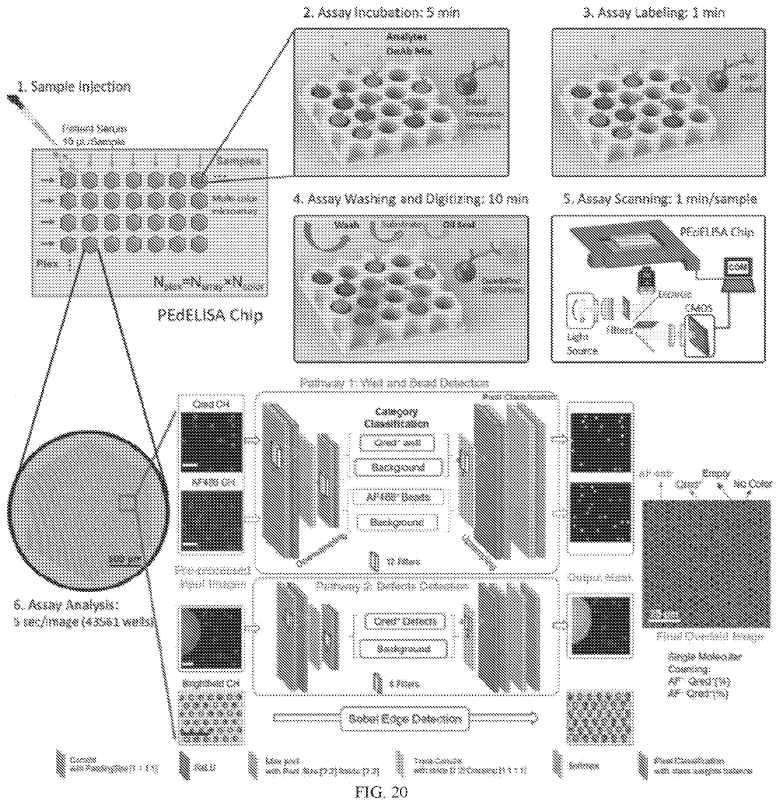

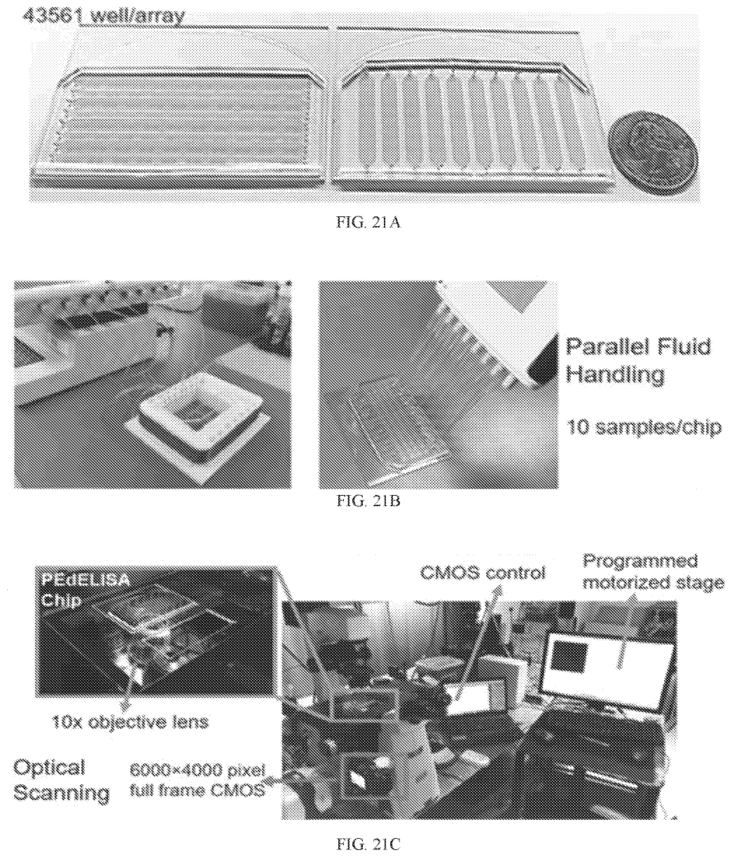

The PEdELISA microarray analysis for the 14-plex analysis used a microfluidic chip fabricated using polydimethylsiloxane (PDMS)-based soft lithography. As described above, the chip contained parallel sample detection channels (10-16) on a glass substrate, each with an array of hexagonal biosensing patterns (FIG. 20). The hexagonal shape allowed each biosensing pattern to densely pack 43,561 femtoliter-sized microwells, which fit into the entire field of view of a full-frame CMOS sensor through a 10× objective lens (FIG. 21).

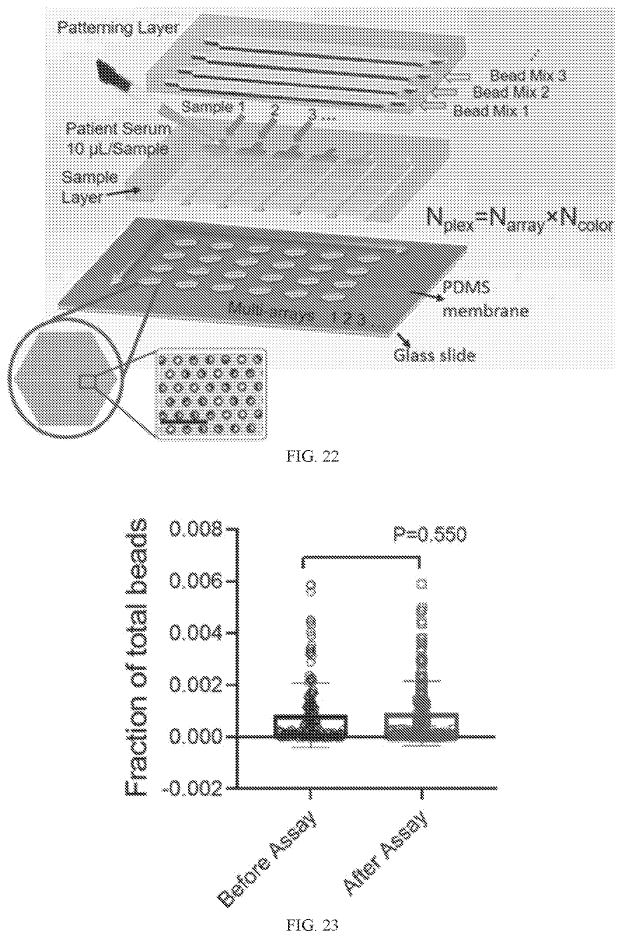

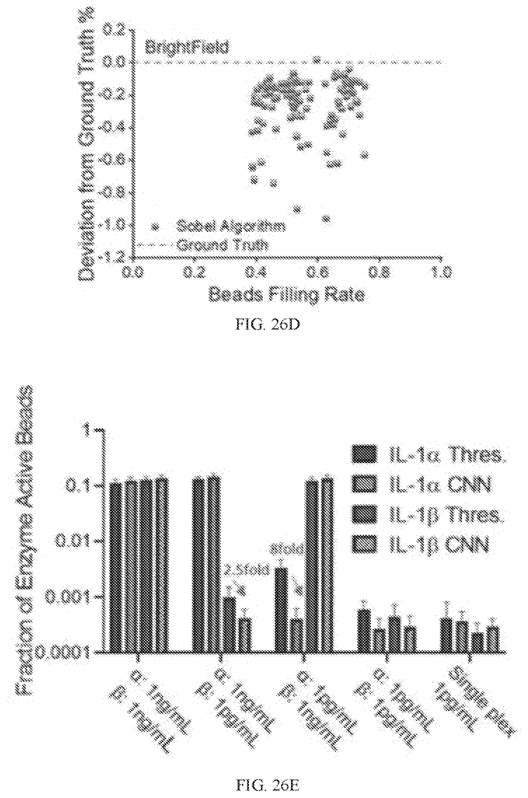

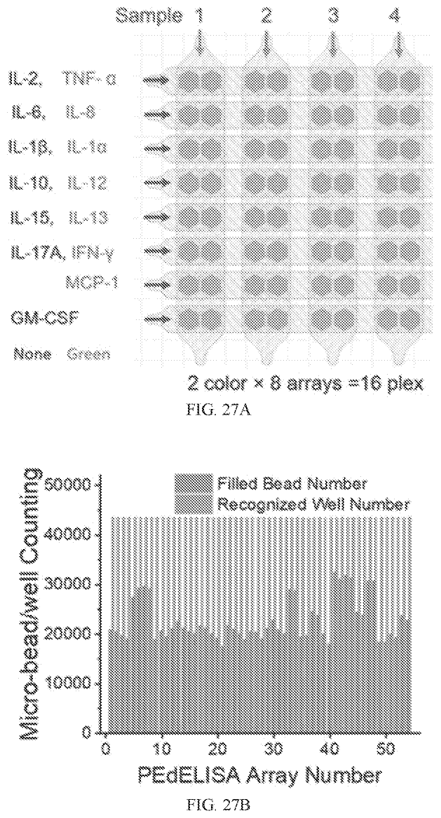

Prior to the assay, magnetic beads (d=2.8 μm) encoded with non-fluorescent color (no color) and with Alexa Fluor® 488 (AF 488) were deposited into physically separated microwell arrays (FIG. 22). The beads were conjugated with different capture antibodies according to their respective colors and locations on the chip. In the design for the 14-plex analysis, the arrangement of 2 colors and 8 arrayed biosensing patterns in each detection channel allowed the PEdELISA microarray chip to detect 2×8=16 protein species (16-plex) at its maximum capacity for each sample loaded to the detection channel (FIG. 27A). Compared with a single color-encoded method, this combination greatly reduced potential optical crosstalk and fluorescence overlap during a signal readout process. The pre-deposition ensured a fixed number of beads to target each biomarker, which allowed more accurate digital counting for each biomarker. It also eliminated bead loss during the conventional partition process and achieved nearly a 100% yield in the signal readout for enzyme active QuantaRed™ (Qred)-emitting beads (“On” beads or “Qred+” beads). The microwell structure (diameter: 3.4 μm and depth: 3.6 m) was designed to generate sufficient surface tension to hold beads in the microwells. This kept false signals resulting from physical crosstalk between the trapped beads at a negligible level (FIG. 23).

Bead flushing test for assessing physical crosstalk False signals were believed to result from misplaced beads during the preparation of the PEdELISA chip. If some of the beads targeting analyte A were accidentally trapped into the microwell arrays of a biosensing pattern to detect analyte B, these misplaced beads would yield either false positive or false negative signals of analyte B, thus confounding the assay (“physical crosstalk” between beads). Previous studies Neelapu, S. S. et al., Nature Reviews Clinical Oncology 2018, 15, 47; Lee, D. W., et al., Blood 2014, 124, 188; Kotch, C., et al., Expert Rev Clin Immunol 2019, 15, 813, all of which are incorporated herein by reference) revealed that choosing an appropriate design for the PDMS microwell allowed surface tension to hold firmly a bead trapped in it even when the entire chip is flipped upside down under prolonged sonication. Guided by these studies, the microwell structure was designed to be 3.4 μm in diameter and 3.6 μm in depth to generate sufficient surface tension to hold beads in microwells (using a permanent magnet could also facilitate the seeding and retention of beads).

To assess the impact of physical crosstalk, a control test was run. The test started with settling AF-488 encoded magnetic beads into one of the sample loading channels of the chip (AF-488 bead channel), and then subsequently settling non-color encoded beads into another channel next to it (non-color bead channel). The untrapped beads were then washed away from these channels, the bead settling layer was peeled off from the multi-array biosensor layer and replaced with the sample loading layer, and harsh flushing was first applied for the AF-488 bead channel and second for the non-color channel at a washing buffer flow rate of 40 uL/min for 15 min. Finally, after sealing the microwells in the channels with oil, a fluorescence microscopy image of the chip was taken and evaluated the number of AF-488 encoded beads invading the non-color bead channel before and after the flushing process. Across 160 independent microwell sites, physical crosstalk was observed at an average bead misplacement percentage of 0.087% out of the total beads originally settled in the non-color bead channel with a 0.0012% standard deviation (FIG. 23). Given that digital ELISA typically forms less than 0.1 antibody-antigen-antibody immune-complexes per bead on average, the false positive signal generated by physical crosstalk was nearly an order less than the typical negative control signal (0.0005-0.001 average molecule per bead) due to non-specific adsorption of proteins.

A unique challenge posed by highly multiplexed digital assays is to provide fast and accurate analysis of fluorescence signals originating from ˜7 million microwells per chip. Additionally, the signal counting process needs to distinguish precisely between images of multi-color bead-filled and empty microwells and to identify signals accurately while subjected to a large fluorescence intensity variance, occasional image defects due to reagent mishandling, and image focus shifts. These challenges make the conventional image processing method with the thresholding and segmentation (GTS) scheme (FIG. 24A) inaccurate, thus requiring human supervision for error correction in handling digital assay images.

Previous studies showed that the use of machine learning algorithms would provide promising solutions to significantly improve the accuracy of digital assay image processing. However, this approach was only applied for single-color images with a small number of microreactors (a few thousand) with 1080×1120 pixels, impractical for high-throughput analysis.

Global thresholding and segmentation vs convolutional neural network visualization FIG. 24 shows the side-by-side comparison between the global thresholding and segmentation (GTS) method and the convolutional neural network (CNN) method in PEdELISA image processing. Both of the methods start from a pre-processing process, including, for example, image cropping, contrast enhancement and noise filtering. The GTS method involved finding and adjusting a global threshold value based on the gray histogram of the image (FIG. 24A, black dash line). This method labeled a microwell site showing a fluorescence intensity level above the threshold value as a positive (“On”) pixel. Then, it applied a 2×2 pixel image erosion mask along the edge to remove the randomly appearing shot noise pixels with intensities above the threshold. As described above, the post-processing process of the GTS method included image dilation and segmentation, “On” microwell/bead counting, error correction and image overlay.

The CNN method, however, runs two signal recognition pathways in parallel, which are pretrained to recognize enzyme active “On” microwells (Red channel, Qred) or beads (Green channel, AF488) versus defects and contaminations using 5,750 labeled images. As a result, this method does not need to predetermine the intensity threshold value required for the GTS method. The CNN method collects three types of raw images (4000×6000 pixels) for each biosensing pattern with 43,561 microwells according to (1) the red fluorescence channel (Qred), (2) the green fluorescence channel (AF488), and (3) the bright-field channel. The Qred- and AF488-channel images were first cropped and pre-processed for noise filtering and contrast enhancement, and then sent directly to the dual-pathway CNN to recognize the enzyme active Qred fluorescence “On” (Qred+) microwell, AF488 fluorescence emitting (AF488+) bead, image defect, and background image features. Then the Qred+ and AF488+ targets were segmented out as the output mask with the defects were removed. The bright-field image was used to recognize both AF488+ and non-color beads and analyzed using the Sobel edge detection method to determine the overall bead filling rate. After post-image processing, the three images were overlaid to determine the numbers of Qred+ microwells and the bead color types (AF488+ or non-color) within them. The fractional population of the Qred+ microwells with respect to the total bead-filled microwells was determined for both the AF488+ (AF+ Qred+ %) and noncolor bead types (AF− Qred+ %) and used to obtain the concentrations of the two different biomarkers. The PEdELISA microarray chip with the current design allowed analysis of up to 16 different analytes for each sample by applying the CNN method-based imaging processing to 8 physically distinct biosensing patterns on it.

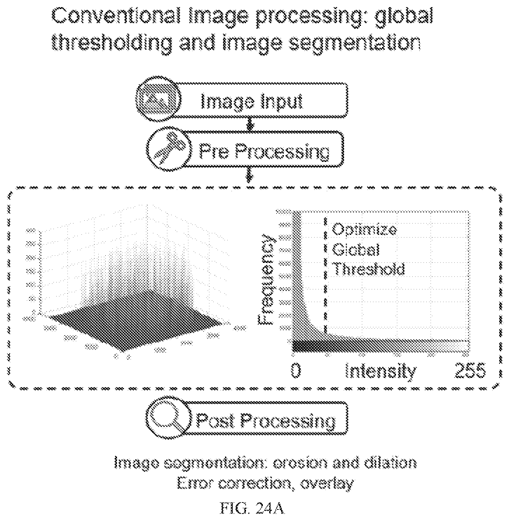

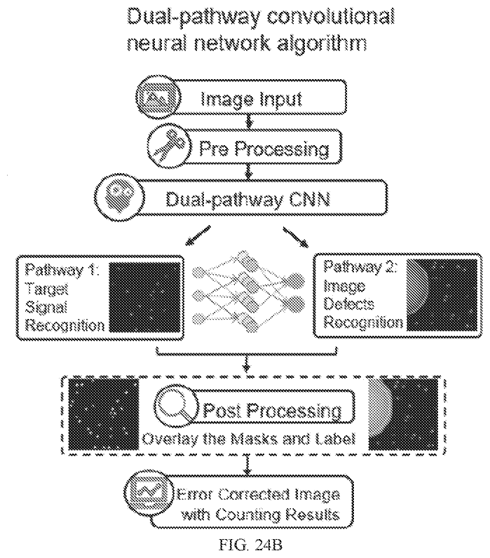

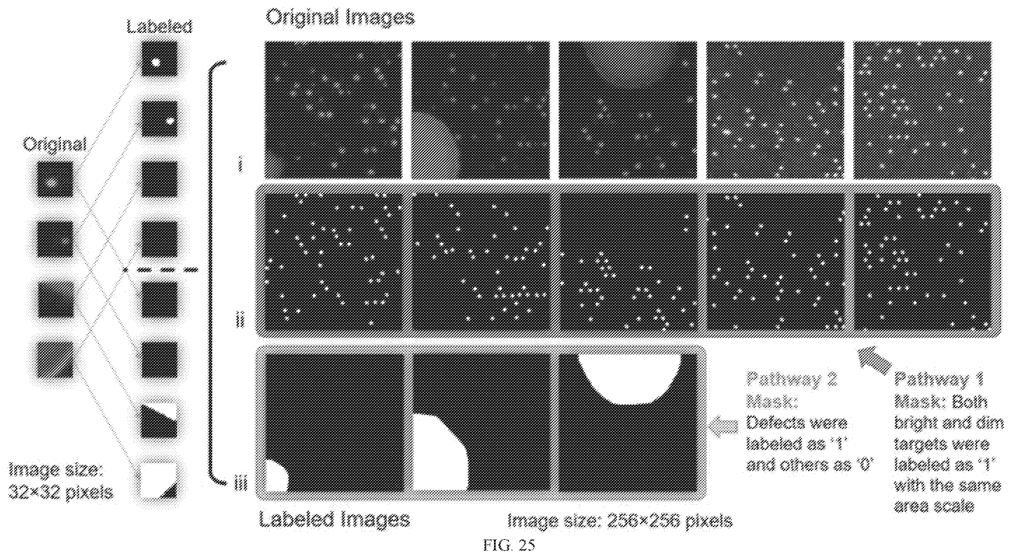

As described above, a novel dual-pathway parallel-computing algorithm based on convolutional neural network (CNN) visualization for image processing was developed to address these challenges. The CNN-based analysis procedure (FIG. 20) includes multi-color fluorescence image data read-in/pre-processing (image crop, noise filtering, and contrast enhancement), microwell/bead image segmentation by pre-trained dual-pathway CNN, post-processing, and result output. The key component, dual-pathway CNN, was pre-trained to classify and segment image pixels by labels of (I) fluorescence “On” (Qred channel) microwells, (II) Alexa Fluor® 488 color-encoded beads (AF488 channel), (Ill) image defects, and (IV) background. The architecture of the network (FIG. 20) separated a downsampling process for category classification and an upsampling process for pixel segmentation. The downsampling process consisted of 3 layers, including 2 convolution layers (4-6 filters, kernel of 3×3) with a rectified linear unit (ReLU), and a max-pooling layer (stride of 2) in between. The upsampling process consisted of a transposed convolution layer with ReLU, a softmax layer and a pixel classification layer. To speed up the training process, an image was divided with 32×32 pixels and classified with labels and then the image pixel size was expanded to 256×256 using a pre-trained network (FIG. 25).

A large intensity variance was found across the optical signals from beads in different microwell reactors. As a result, the intensity-based labeling of microwells could lead to recognition errors due to microwells with bright beads being misrecognized to have larger areas with more pixels than those with dim beads. Given that all microwells are lithographically patterned to have an identical size, each microwell was labeled using the same pre-fixed area scale (octagon, r=3 pixel for microwell, disk, r=2 pixel for bead) regardless of their image brightness for correct machine recognition. The majority of pixel labels are used for the background (Label IV) with no assay information in typical digital assay images. The inverse frequency weighting method was used to further enhance the classification accuracy, giving more weights to less frequently appearing classes identified by Labels (I), (II), and (III).

Training details of the dual-pathway convolutional neural network FIG. 25 shows the training process of the dual-pathway CNN algorithm, which involved data set preparation and 2-step neural network training. Pre-stage neural networks trained with a data set of 3,000 representative locally-cropped (32×32 pixel) images were first developed. For each of these images, two types of images (6,000 in total) only showing the labeling information, which are called “masks” were generated by thresholding and manual labeling (FIG. 25, Labeled). In the pre-stage mask for Pathway 1, all the target pixels representing “On” (Qred+) microwell sites and other sites (defects or background) were labeled as ‘1’ and ‘0’, respectively. In the pre-stage mask for Pathway 2, the defects and other sites were labeled as ‘1’ and ‘0’, respectively. The pre-trained networks were able to identify and segment the labeled objects to some degree of accuracy (i.e., 60-80% “On” wells). But they still lacked the accuracy to extract more in-depth features of the object, such as its shape and fluorescence intensity variations of the objects, and other defects that were hard to identify with the small-scale images.

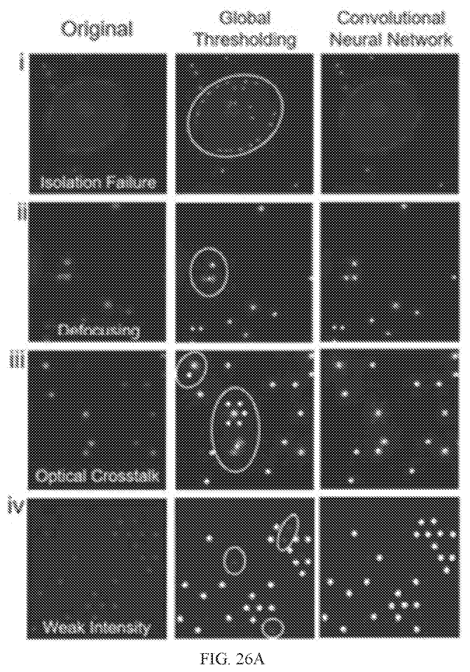

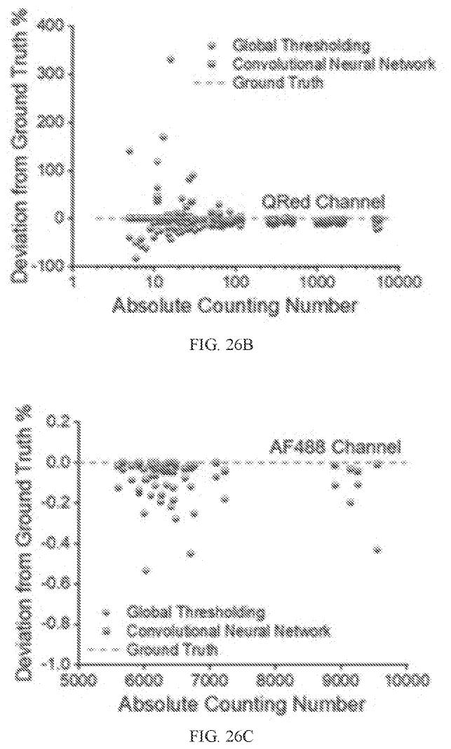

To further improve the accuracy, three second-stage parallel CNN networks were built to identify AF 488+ pixels, Qred+ pixels, and defects that were trained with 256-256-sized images (around 200 images per network). The labeling masks to be used to train the second-stage networks were generated by the pre-stage neural networks with human correction (FIG. 25, ii). By taking into account that the sizes of lithographically patterned microwells and beads were predetermined, the second-stage networks were trained to accurately recognize these objects independent of their image intensity variations. Additionally, 30 images were selected for each error source caused by optical crosstalk (FIG. 26A), the intensity threshold was carefully determined to set a clear boundary between adjacent microwells for each of these images, and error-free second-stage labeling masks were generated.

As for training the defect recognition-network, the image brightness and contrast were first enhanced to recognize even low-intensity regions. Image dilation was then applied for the defect location to ensure the generated mask area was large enough to cover the entire defect area as shown in FIG. 25, iii. Here, only defect locations were labeled as ‘1’ and all others were labeled as ‘0’. The region labeled as ‘1’ was eventually removed from the total analyzed area to eliminate it from the fluorescence signal counting.

The neural network contained 5 classes: Qred+ class, AF488+ class, Qred defect class. Qred background class, and AF488 background class. Before training the neural network with the labeling masks above, weight information was also added to each class to further enhance the pixel identification accuracy. The inverse frequency weighting method was used which gives more weights to less frequently appearing classes. The class weight was defined as

$$Classweight = \frac{{N}_{image total pixels}}{{N}_{class pixels}} \qquad ( 10 )$$

where N_{image total pixels} is the number of total image pixels of 256×256=65,536, and the N_{class pixels} is the number of pixels for each class. This class weighting strategy was added into the neural network training process because the number of Qred+ or AF488+ class pixels were significantly smaller than the number of their total background pixels.

In contrast to a previously reported study, the number of convolution layers and filters (depth of network) was greatly reduced for high speed processing. The algorithm employed much fewer labels and features required for imaging processing than those for other typical CNN applications, such as autonomous driving. The unique feature of the algorithm used here is the ability to run two neural networks in parallel for two detection pathways: one for assay targets (e.g. microwells, beads, and fluorescence signals) and the other for defects. This allowed the imaging processing to achieve high speed while maintaining good precision. As a result, it only took ˜5 seconds (CPU: Intel Core i7-8700, GPU: NVIDIA Quadro P1000) to process two-color channel data for two 6000×4000 pixel images which contain 43561 micro-reactors.

To validate the effectiveness of the dual-pathway CNN method developed in this work, it was compared with that of the standard method based on global thresholding and segmentation (GTS). FIG. 26A shows representative two-color-channel images causing errors to the image labeling and signal counting of the GTS method. These errors are corrected by the CNN method. For example, false signal counting is commonly derived from chip defects or poor labeling reagent confinements within individual microwell reactors due to the local failure of oil sealing. Defocusing can cause two neighboring microwells to be dilated with each other. Highly bright Qred fluorescence from an “On” microwell can cause secondary illumination to light up neighboring microwells. This results in “optical crosstalk” between neighboring microwells, which causes the false counting of secondarily illuminated microwells as “On” sites. The uneven illumination of excitation light causes the failure of recognizing dim AF-488 encoded beads (recognized as non-color beads).

In the CNN training process, a large number of images were collected for each error source and used to train the neural network to achieve results similar to those from manual counting with the human eyes. The following equation was applied to evaluate the error in terms of deviation to the ground truth (%):

$$Deviation ( \% ) = \frac{{N}_{CNN or GTS} - {N}_{TP}}{{N}_{TP}} \times 100 \% \qquad ( 11 )$$

where N_{CNN or GTS} is the number of microwell or bead counted either by CNN or GTS method respectively, N_{TP} is the number of true positives determined by human labeling. The global threshold value was adjusted based on the gray histogram of the image (FIG. 24). The human labeling process included the pre-processing with the GTS method together with human correction to obtain the ground truth, which was validated by the conventional sandwich ELISA method, as described above (FIG. 7F).

In counting enzyme active microwells with the Qred channel, it was observed that the deviation percentage from ground truth varied with the number of the counted “On” (Qred+) microwells, which is proportional to the analyte concentration. Each data point in FIGS. 26B-D was taken for a hexagonal-shaped biosensing pattern (FIG. 20) that contains 43561 microwell arrays with an average bead filling rate of 55.1%. In these data, the number of Qred+ microwells ranged from 1 to 10000 (FIG. 26B). At higher analyte concentrations (NQ_{red}>100), both of the methods achieved reasonably high accuracy with a deviation to the ground truth of 3.92% (CNN) and 9.96% (GTS). However, at the lower concentrations (NQ_{red}<100), this deviation became significant (CNN: 5.14% GTS: 71.6%). The larger error of the GTS scheme was attributed to the false counting of regions contaminated with fluorescent reagents and the miscounting of Qred+ microwells of low fluorescence intensity. Thus, the dual-pathway CNN greatly improved the accuracy of the PEdELISA image processing and replacing the thresholding method with the CNN method eliminated the need for human supervision to correct the significant errors in the low concentration region.

In counting color-encoded magnetic beads with the AF-488 channel, it was found that the deviation was very small (CNN: 0.021%, GTS: 0.161%). The deviation was suppressed by the little spectral overlap between AF488 and Qred, channels and the high image contrast that was intentionally created between AF-488 and non-color encoded beads (FIG. 26C). Some miscounting under the uneven spatial distribution of illumination light intensity and the spherical aberration of objective lens over the entire field of view still contributed to the deviation. The CNN method achieved a nearly 8-fold improvement of accuracy. Counting the total number of beads (both no color and fluorescence color-encoded ones) with brightfield images using a customized Sobel edge detection algorithm yielded an average deviation to the ground truth of 0.256% as shown in FIG. 26D.

To verify the ability to suppress optical crosstalk in the multiplexed assay incorporating the CNN method, a 25% fetal bovine serum (FBS) sample spiked with two different cytokine species, IL-1α (AF488 encoded) and IL-1β (non-color encoded), of 1000-fold concentration difference was prepared. Optical crosstalk can become problematic especially in multiplexed analysis, where the quantity of one biomarker can be serval orders of magnitude higher than those of other biomarkers in the same sample. A slightly false recognition can give a significantly higher value of biomarker concentration than its true value. FIG. 26E shows the comparison between the conventional GTS method and the CNN method. False recognition was greatly reduced by the CNN method and it was verified that 1 pg/mL of IL-1α or β will not interfere even when the other protein reaches 1 ng/mL. Furthermore, single-plexed measurements of 1 pg/mL of IL-1α and IL-1β were performed, which gives “true” concentration values while eliminating optical crosstalk. The single- and dual-plexed measurements both yielded statistically similar results with the CNN method (two-tailed unequal variance t-test, IL-1α P=0.253; IL-1β=0.368), which proved the accuracy of this method even at the presence of strong optical crosstalk.

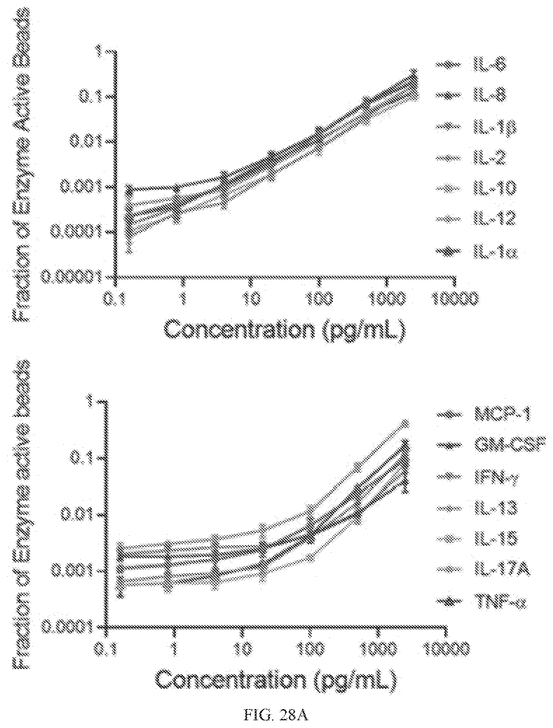

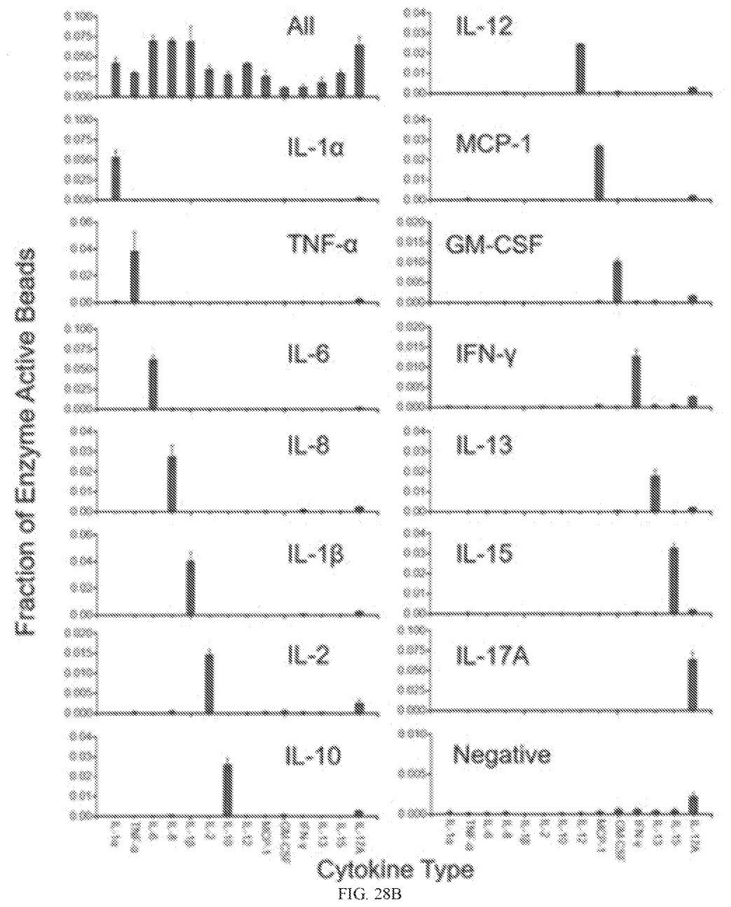

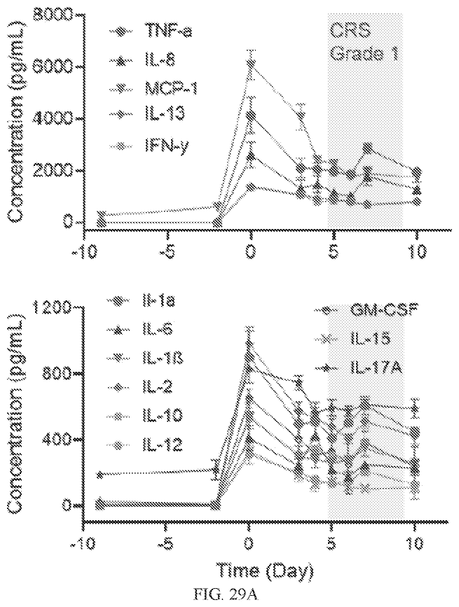

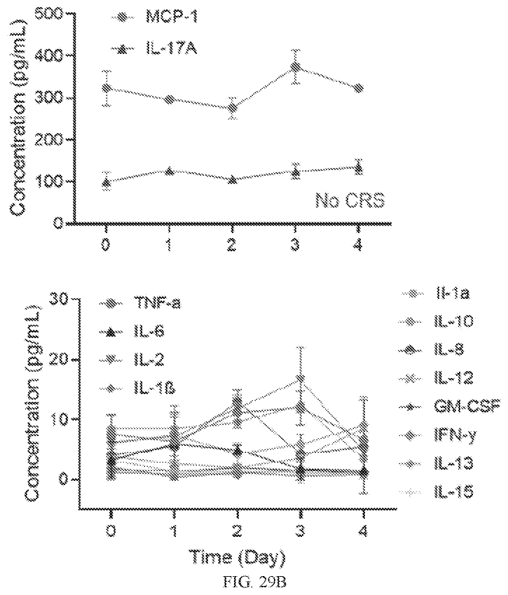

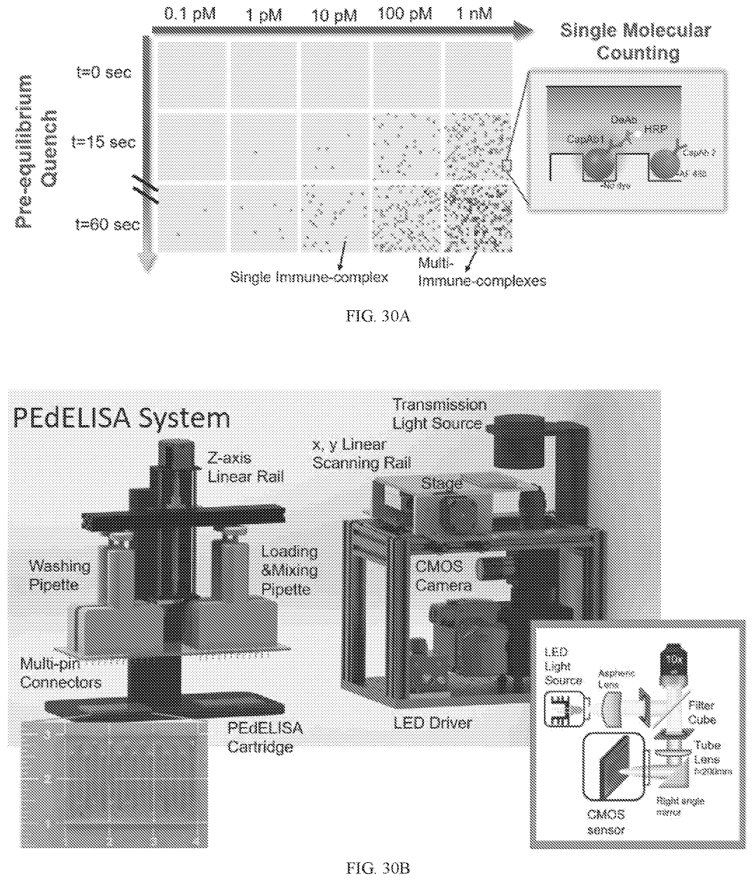

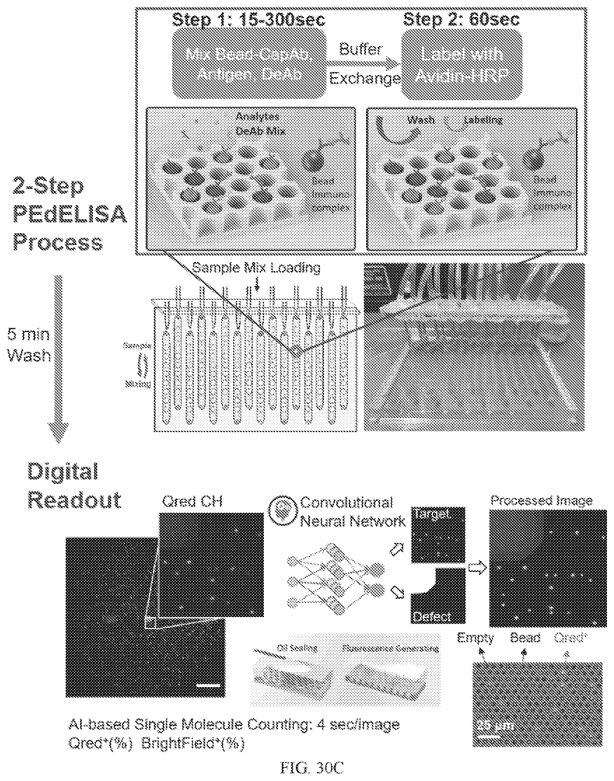

Using 2-color encoded (AF488, non-color) magnetic beads with 8 physically separated microarrays, a microfluidic chip was designed to detect 14 cytokines (up to 16-plex) simultaneously (see chip design in FIG. 27). FIG. 28A shows standard curves obtained from PEdELISA microarray analysis with CNN image processing for cytokines ranging from 0.16 pg/mL to 2.5 ng/mL in 25% FBS. Here, the measurement output was the fraction of the number of enzyme active (Qred+) beads to the total number of beads used for assaying the particular analyte. This fraction was directly correlated to the analyte concentration. The assay was performed for a system at the early state of a transient sandwich immune-complex formation reaction process with a 5-min incubation period, followed by a 1-min enzymatic labeling process. The reaction conditions have been optimized to match all cytokine biomarkers within the clinically relevant range, and a linear dynamic range of three orders of magnitude was achieved in general. Table 5 summarizes the values of the limit of detection (LOD) and limit of quantification (LOQ) of the assay for each cytokine. The antibody-antigen affinity affected the LOD of the assay, and it varied across the detected cytokine species. As a result, different LOD values were obtained for these cytokines even if the capture antibody-conjugated beads were prepared by the same protocol regardless of the analyte types. The LOD value tended to decrease with an increasing incubation period. Although the assay was performed with a short incubation period of 5 min, the LOD was found to be still below 5 pg/mL (with IL-1β reaching the lowest 0.188 pg/mL) after optimizing the detection antibody mixing ratio and the enzyme labeling concentration.

TABLE 5
Limit of detection (LOD), Limit of Quantification (LOQ) and standard root mean
square coefficient variance (RMS CV) summary of 5-min, 14-plex PEdELISA.

| Cytokine | TNF-α | EL-6 | IL-8 | IL-1β | IL-2 | IL-10 | IL-12 |
|---|---|---|---|---|---|---|---|
| Assay Blank + | 0.0580 | 0.0456 | 0.0920 | 0.0148 | 0.0222 | 0.0217 | 0.0493 |
| 3σ (%) | | | | | | | |
| Assay Blank + | 0.1355 | 0.1168 | 0.1820 | 0.0404 | 0.0601 | 0.0584 | 0.1109 |
| 10σ (%) | | | | | | | |
| LOD^{†} 5-min | 2.195 | 2.667 | 2.033 | 0.188 | 0.535 | 0.210 | 0.613 |
| (pg/mL) | | | | | | | |
| LOQ* 5-min | 12.80 | 12.21 | 11.54 | 0.696 | 11.24 | 5.661 | 4.996 |
| (pg/mL) | | | | | | | |
| Assay standard | 16.0 | 6.30 | 14.6 | 5.19 | 15.3 | 9.96 | 14.1 |
| RMS CV^{‡} (%) | | | | | | | |
| Cytokine | IL-1α | MCP-1 | IL-13 | IL-15 | IL-17A | INF-γ | GM-CSF |
| Assay Blank + | 0.0527 | 0.1557 | 0.0706 | 0.0518 | 0.3675 | 0.2283 | 0.2139 |
| 3σ (%) | | | | | | | |
| Assay Blank + | 0.1240 | 0.3769 | 0.1758 | 0.1299 | 0.6641 | 0.3566 | 0.2896 |
| 10σ (%) | | | | | | | |
| LOD^{†} 5-min | 0.349 | 3.406 | 1.733 | 3.206 | 4.120 | 3.448 | 7.797 |
| (pg/mL) | | | | | | | |
| LOQ* 5-min | 2.674 | 47.83 | 18.296 | 57.944 | 43.423 | 62.66 | 35.33 |
| (pg/mL) | | | | | | | |
| Assay standard | 13.1 | 18.8 | 10.1 | 20.6 | 11.1 | 12.6 | 10.7 |
| RMS CV^{‡} (%) | | | | | | | |

^{†}LOD was determined by concentration from the reagent blank signal + 3σ.
*LOW was regent blank signal + 10σ.
^{‡}RMS CV was determined by the root mean square average signals from 20, 100, and 500 pg/mL assay standard with typical three day-by-day repeats and 2 on-chip repeats.

The level of antibody cross-reactivity was further assessed among 14 cytokines in 25% FBS. FIG. 28B shows the assay results for sera spiked by all, one, or none of the recombinant cytokines of 14 species, namely “all-spike-in,” “single-spike-in,” and “no-spike-in” samples. More than 100 times lower background signal was observed from the no-spike-in (negative) sample than from the all-spike-in sample across the 14 cytokines (except IL-17A for which there is a slightly higher background due to the more active binding between its capture and detection antibodies). The signal-level variation across the 14 cytokines at the same concentration from the all-spike-in and single-spike-in samples could be derived from the different levels of analyte-antibody affinity for these cytokines. A similar trend was also observed in the variation of the LOD values for the cytokines from the curves in FIG. 28A. The signal from each of the 14 single-spike-in samples manifested a high level of specificity to the target analyte. This verified that the multiplexed assay measurements caused negligible cross-reactivity between each cytokine analyte and other capture and detection antibodies that should not couple with it.

Finally, the 14-plex PEdELISA microarray analysis was applied to the longitudinal serum cytokine measurement from patients receiving CAR-T cell therapy. CAR-T therapies have demonstrated remarkable anti-tumor effects for treatment-refractory hematologic malignancies. Unfortunately, up to 70% of leukemia and lymphoma patients who receive CAR-T therapy experience cytokine release syndrome (CRS). CRS is a potentially life-threatening condition of immune activation caused by the release of inflammatory cytokines (e.g., IL-6, TNF-α, and others). CRS initially causes fevers and other constitutional symptoms that can rapidly (i.e., within 24 hours) progress to hypotension and organ damage requiring intensive care. Previous studies have shown the measurement of a panel of cytokines can indicate the early onsite of severe CRS. Thus, the way of intervening CRS could be significantly improved by the multiplex PEdELISA microarray analysis.

To demonstrate the clinical utility of the assay technology, the assay was run for two CAR-T patients, one who experienced up to grade 2 CRS and one who did not experience CRS in the first few days post CAR-T infusion. The total sample-to-answer time achieved was 30 min for the entire 14-plexed measurement including the sample incubation (5 min), labeling (1 min), washing/reagent confining (10 min), and image scanning/analysis (14 min) processes. FIG. 29A shows that Patient 1 developed CRS on day 4 that persisted until day 9. Significant elevations were found for all assayed cytokines on Day 0 in comparison to their baseline levels on Day 2 and Day 9. Interestingly, the spike on Day 0 was not due to the CAR-T cells, as the blood sample was taken prior to CAR-T infusion. Typically, CRS patients exhibit a high IL-6 concentration within their blood. However, Patient 1 manifested a significantly higher level of TNF-α. This suggested biological heterogeneity in the pathogenic cytokine profiles of patients who develop CRS. A similar analysis was also conducted for a patient who did not develop CRS (FIG. 29B). An increase in IL-2 and a relatively high level of IL-17A was recorded for this patient, while other cytokines showed no significant changes throughout the analysis. Presumably, normal CAR-T cell expansion was taking place in the patient's body.

A highly multiplexed digital immunoassay platform, the PEdELISA microarray, employed a unique combination of spatial-spectral encoding and machine learning-based image processing on a microfluidic chip. The positional registration of on-chip biosensing patterns, each with more than 40,000 microwell reactors confining sample sub-volumes, fluorescence-encoded analyte-capturing beads, and assay reagents, enabled 14-plex cytokine detection for 10 μL of serum with high sample handling efficiency, small reagent loss, and negligible sensor cross talk. The signal processing and analysis of the 14-plexed PEdELISA microarray analysis employed a parallel computing CNN-based machine-learning algorithm. This algorithm achieved autonomous classification and segmentation of image features (e.g. microwells, beads, defects, backgrounds) at high throughput (I min/analyte). Notably, it yielded 8-10 fold higher accuracy than the conventional GTS-based algorithm without any human-supervised error correction.

Microfluidic Chip Fabrication and Spatial-Spectral Encoding The first step of the PEdELISA microarray chip fabrication involved the construction of three different PDMS layers (FIG. 22). The first PDMS layer had arrays of hexagonal biosensing patterns with microwell (d=3.4 μm) structures (multi-array biosensor layer). The second PDMS layer had bead settling flow channels of 2500 μm in width, 70 μm in height, and 65 mm in length (bead setting layer). The third PDMS layer had channels of 4500 um in width, 90 μm in height, and 30 mm in length for the detection of analytes in loaded samples (sample detection layer). SU-8 molds were first constructed for the three PDMS layers on separate oxygen plasma treated silicon wafers by standard photolithography. This process involved depositing negative photoresist (SU-8 2005, SU-8 2050, MicroChem) layers at different spin coating speeds to form the designed thicknesses for the PDMS microstructure patterns. Subsequently, a precursor of PDMS was prepared at a 10:1 base-to-curing-agent ratio. To construct the multi-array biosensor layer, a thin PDMS precursor film (˜300 μm) was deposited onto the microwell-patterned SU-8 mold by spin coating, baked overnight (60° C.), and attached to a pre-cleaned 75×50 mm glass substrate through oxygen plasma treatment. Both the bead settling layer and the sample detection layer were made by pouring the PDMS precursor over the other SU-8 molds in a petri dish and then baked overnight (60° C.).

The second step involved the settlement of beads in the microwells of each hexagonal pattern on the multi-array biosensor layer. The bead setting layer was first aligned and attached to the multi-array biosensor layer on the glass substrate. Then, 7 sets of a 25 uL mixture of AF488 encoded beads (anti-cytokine 1) and non-color encoded beads (anti-cytokine 2) at the concentration of 1 mg/m were prepared in vials for the 14-plex detection. This was followed by loading each of the 7 mixtures into one of the microfluidic channels in the bead settling layer (FIG. 22). After waiting for the beads to settle in the microwells for 5 min, the bead settling channels were washed with 200 uL PBS-T (0.1% Tween20) to remove the unstrapped beads thoroughly. At this step, it was ensured that the microwells were filled with the beads at a sufficient rate (typically above 50%) using an optical microscope. If not verified, the bead mixture solution was reloaded and washed again.

The third step involved the assembly of the chip with the multi-array biosensor and sample detection layers. After the bead setting channels were dried by sucking out the washing buffer using a pipette, the bead settling layer was removed from the multi-array biosensor layer and replaced with the sample detection layer. Here, the sample detection layer was aligned and attached to the multi-array biosensor layer so that its channels were oriented perpendicular to the direction of the channels of the bead settling layer. The sample detection channels were then slowly loaded with Superblock buffer (0.05% Tween20) to passivate the PDMS surface and incubate the whole chip for at least 1 hour before the assay to avoid non-specific protein adsorption. The sample detection layer was punched to form inlets and outlets for its channels. The chip was tape cleaned and covered before the assay usage. Finally, serum samples were loaded to the sample detection channels from their inlets (FIG. 22).

### Example 8

### Multiplexed Cytokine Monitoring of CAR-T Cell Therapy-Associated CRS

PEdELISA was used to monitor the cytokine profiles of hematological cancer patients showing different levels of CRS symptoms after CAR-T cell therapy following a pre-approved sample collection protocol. CRS or cytokine storm frequently accompanies various diseases, including cancer immunotherapy, macrophage activation syndrome in autoimmune disease, severe sepsis, or the recent global outbreak of the novel coronavirus pneumonia (COVID-19). It can rapidly evolve (i.e., within 24-48 hours) from manageable constitutional symptoms (grade 1) to more severe forms (grade 2-4), for which rapid and sensitive serum cytokine measurements could direct urgent interventions. For one of the most severe CRS patients (Patient 06), a near-real-time cytokine profile analysis was performed within 2 hours after blood samples were freshly drawn with a sample-to-answer time of ˜30 minutes (FIG. 10A). To ensure the highest accuracy and sensitivity for these clinical measurements, the total incubation time was chosen to be 300-sec (Step 1)+60-sec (Step 2) in the 2-step assay format.

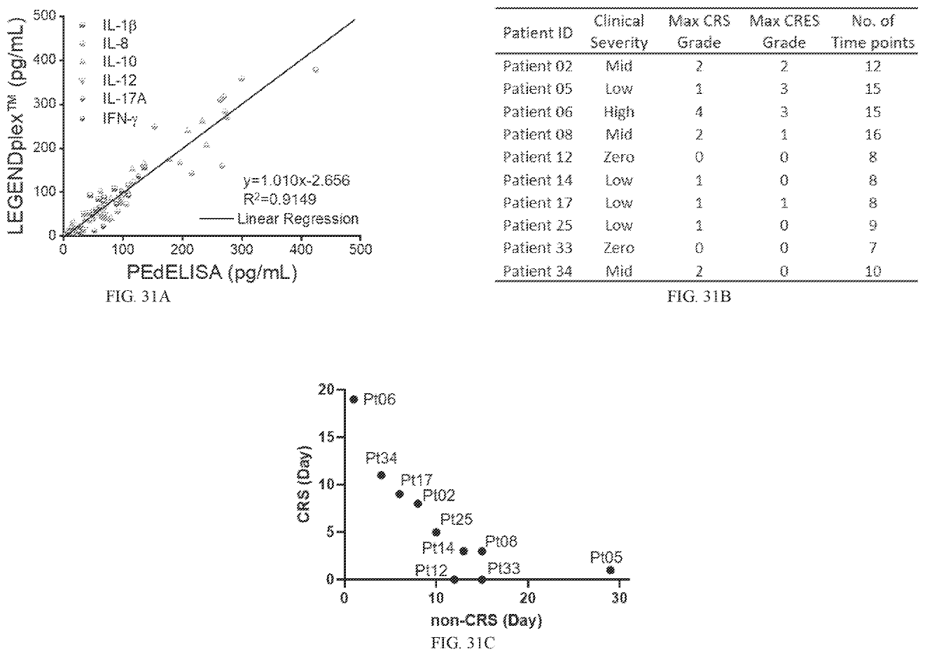

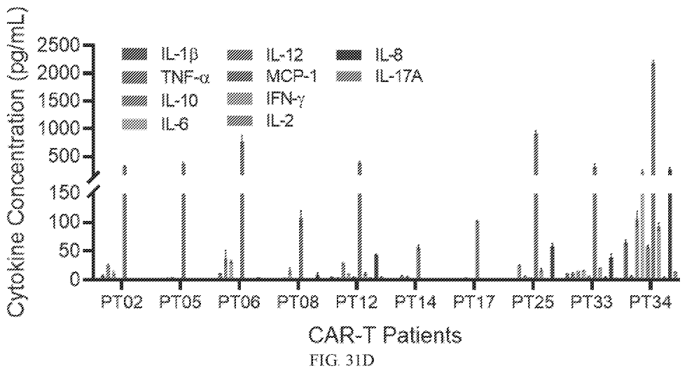

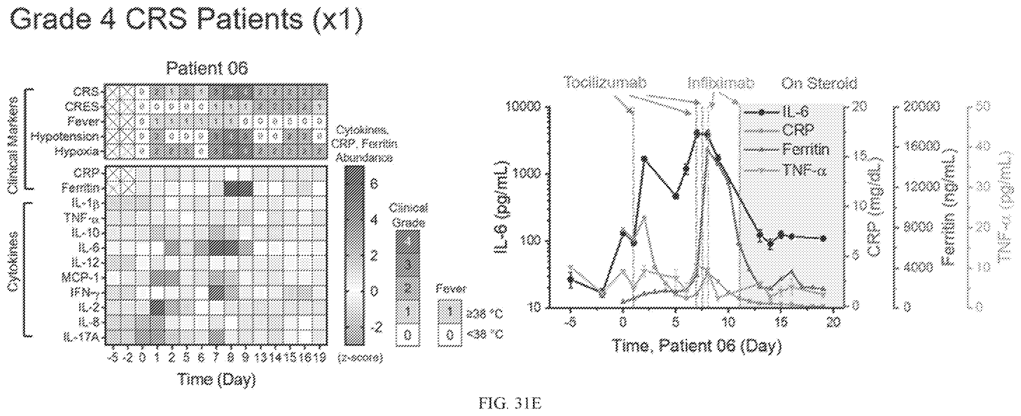

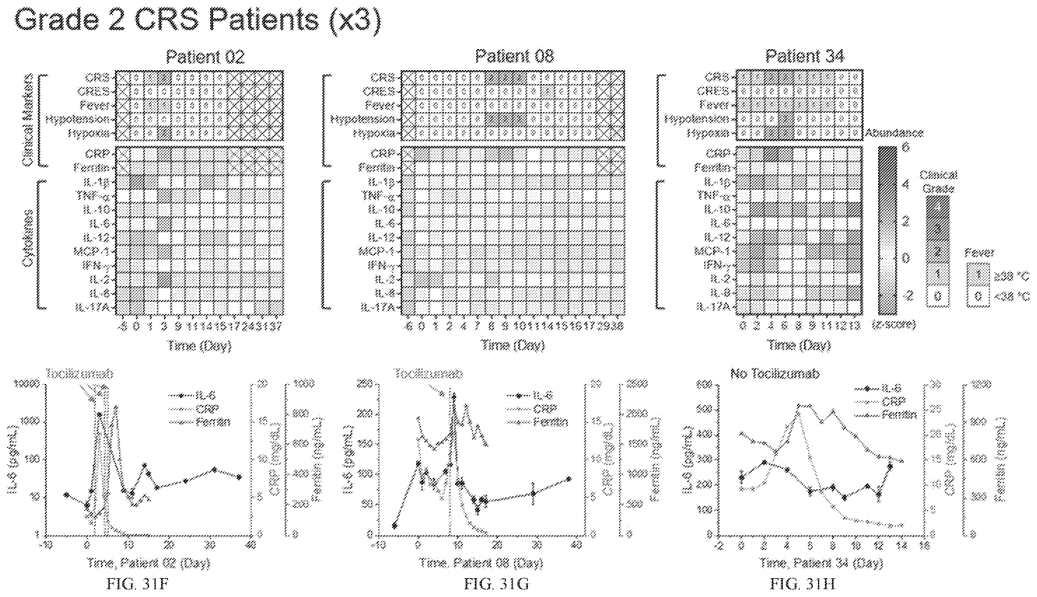

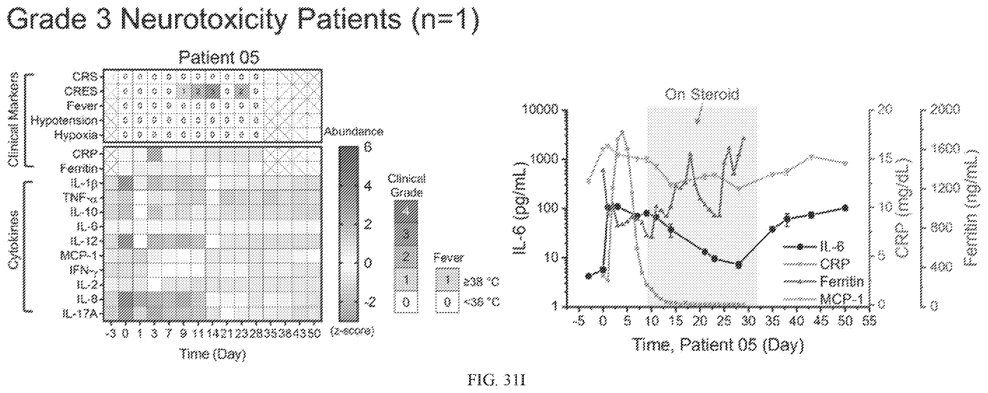

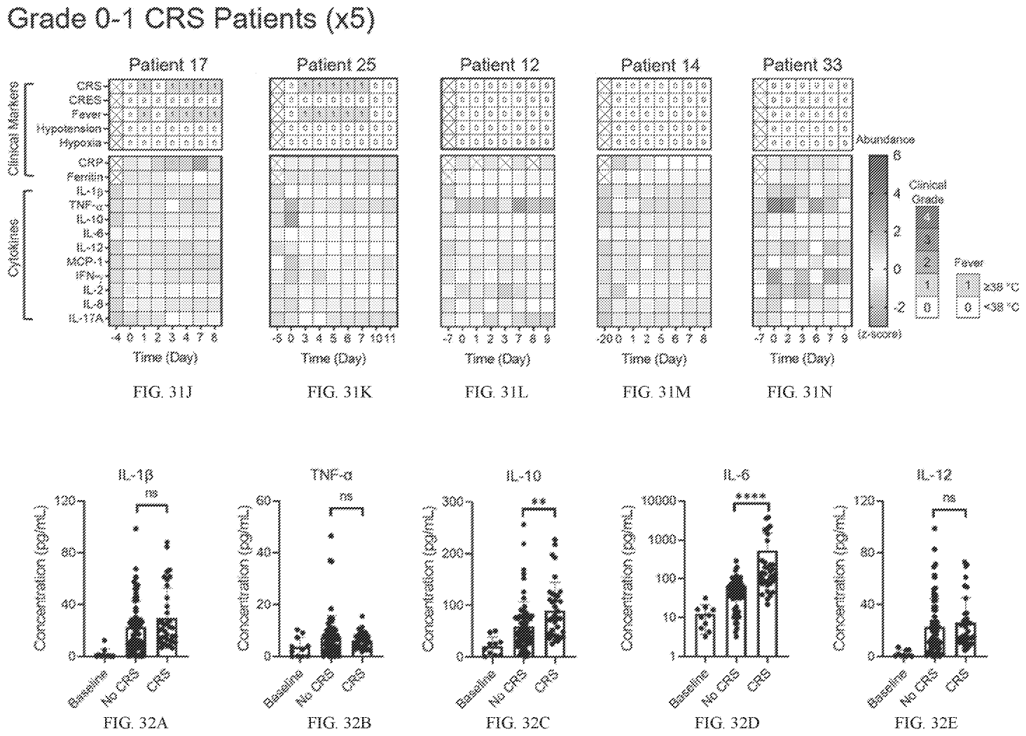

In addition to spike-in tests of known analytes as described above, 20 banked serum samples were assayed from three different patients with unknown concentrations of IL-1β, IL-8, IL-10, IL-12, IL-17A, and IFN-γ using both PEdELISA and a commercial multiplex assay, LEGENDplex™ (BioLegend). The results of these two assay methods showed a strong linear correlation (R²=0.915), providing additional validation of the 2-step PEdELISA assay for multiplex cytokine detection (FIG. 31A).

The patients studied showed a range of CRS severity, including high- (Patient 06), mid- (Patient 02, 08, and 34), and low-grade (Patient 05, 14, 17, and 25), as well as no CRS (Patient 12 and 33) after CAR-T cell infusion (FIGS. 31B-31C). Patients exhibited heterogeneous cytokine profiles immediately prior to CAR-T cell infusion (FIG. 31D). To capture the patients' dynamic immune responses to CAR-T cell therapy and immunomodulatory interventions, blood samples were collected and processed for serum daily except for weekends (Saturdays and Sundays). FIGS. 31E-31N present the heat maps of longitudinal cytokine profiles for the patients together with clinical indicators (CRS. CAR-T-cell-related encephalopathy syndrome (CRES) grade, fever, hypotension, and hypoxia), and clinical inflammatory markers (C-Reactive Protein (CRP), and Ferritin). Each marker's concentration values are normalized by the Z score, and the data are grouped according to the severity of the patients' CRS conditions. For the patients receiving immunomodulatory treatments, FIGS. 31E-31I provide longitudinal plots of the quantitative values of selected cytokines with higher relevance to the treatments next to the heat maps. These plots show the effects of anti-inflammatory drugs, including tocilizumab (anti-IL-6R) and infliximab (anti-TNF-α), and corticosteroid (dexamethasone) on cytokine profiles. The treatments for these patients were chosen solely based on clinical criteria (e.g., CRS grades), not based on serum cytokine data.

For Patient 06, who initially had a high disease burden, the time to initial onset of CRS was as short as 13.5 hours. Several biomarkers, such as MCP-1, IL-1β, IL-2, and IL-8 levels rose rapidly and reached peak values of MCP-1=2947 pg/mL, IL-1β=75.3 pg/mL, IL-2=39.72 pg/mL, and IL-8=415 pg/mL within 24 hours after CAR-T infusion, which correlated with the patient's grade 2 CRS, accompanying fever (39.3° C.) on Day 1 (FIG. 31E). A continuous rise of IL-6 was also observed transiently after the first tocilizumab administration (Day 1) from 89.9 pg/mL (Day 1) to its peak level of 1676 pg/mL through Day 2. Patient 06 later developed grade 3-4 life-threatening CRS on Day 6-9 and was readmitted to the ICU. During this period, IL-6 and IFN-γ approached extremely high levels (IL-6=4383 pg/mL IFN-γ=224.7 pg/mL), as indicated by the red colors on the heat map. Several other cytokines, such as MCP-1, IL-1β, IL-8, IL-10, and IL-17A, also showed second peaks, despite the administration of multiple immunosuppressive agents. Interestingly, a significant rise in TNF-α and IL-2 was not seen during the second CRS peak. On Day 11, the patient was diagnosed with grade 3 CRES and was treated with steroids (dexamethasone). Overall, the cytokine levels significantly decreased after these treatments.

Regarding grade 2 CRS patients. Patient 02's IL-6 showed a temporarily large increase following tocilizumab administration (FIG. 31F). The patient received the first dose on Day 2 with grade 1 CRS, and a peak (1546 pg/mL) was detected for IL-6 on Day 3. Similarly, Patient 08 (FIG. 4H) received tocilizumab on Day 8 with grade 2 CRS, and a peak (228.5 pg/mL) was detected for IL-6 on Day 9. Patient 34 did not receive tocilizumab and the level of IL-6 consistently stayed in the narrow range between 300 and 500 pg/mL with no dynamic changes throughout the CRS period for this patient.

Patient 05 did not develop CRS after the CAR-T cell infusion, although a slight elevation of all four cytokines was observed on Day 0 and 1 (FIG. 31I). However, Patient 05 developed prolonged neurotoxicity starting from Day 8 and was treated with steroids from Day 9 to Day 33. During this period, all ten cytokines stayed at low levels. After steroids were discontinued, Patient 05's IL-6 and MCP-1 levels rose from Day 35 to Day 50, and grade 1 neurotoxicity relapsed.

FIGS. 31J-31N summarize the data for the patients who developed mild CRS (grade 1) or no CRS symptoms. Overall, the cytokine levels were low relative to those of patients from the other groups. The heat maps of these patients generally show small fluctuations of cytokine levels, although some minor peaks were observed in the first few days after the CAR-T cell infusion. Notably, the red scale of the heat map of Patient 33 represents absolute values of TNF-α and IL-2 levels as small as 23.6 pg/mL and 13.3 pg/mL, respectively.

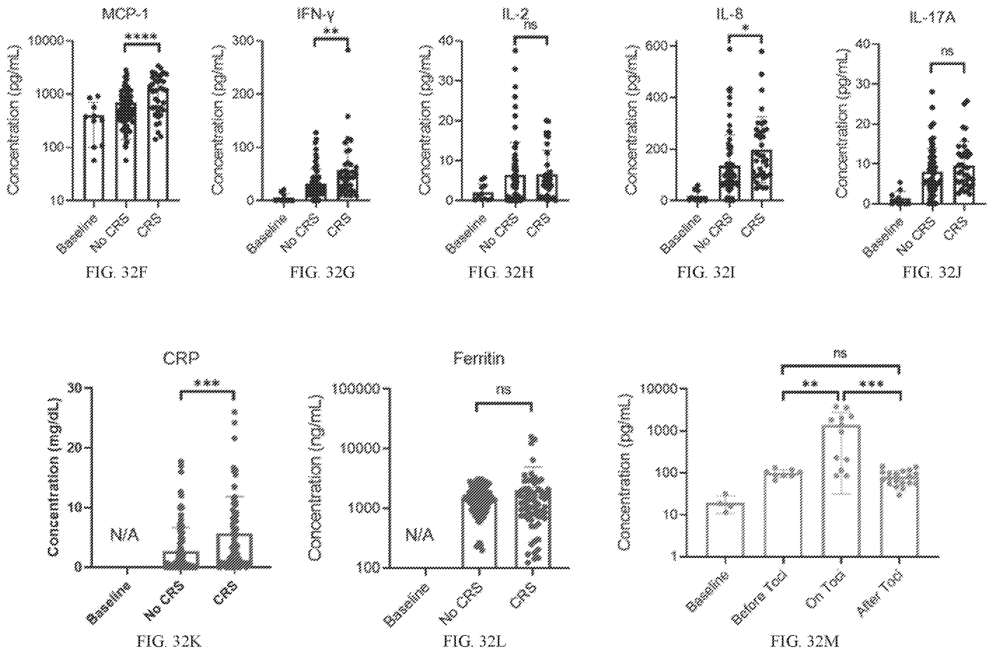

FIG. 32 shows longitudinal cytokine data in FIG. 31, all combined for the ten CAR-T patients, sorted into non-CRS and CRS (grade 1 or higher) groups. A statistically significant difference was seen between the two groups for IL-6 (P<0.0001), IL-8 (P<0.05), IL-10 (P<0.01), MCP-1 (P<0.001), IFN-γ (P<0.01) and CRP (P<0.001), including data at time points when CRS patients were treated with steroids or other immunosuppressive agents. IL-6 data was also analyzed for the three patients who received tocilizumab treatment (FIG. 32M). “On Toci” represents 0-3 days after tocilizumab administration and “After Toci” represents >3 days after tocilizumab administration. Significant elevations of IL-6 were observed shortly after treatment with tocilizumab (P<0.01).

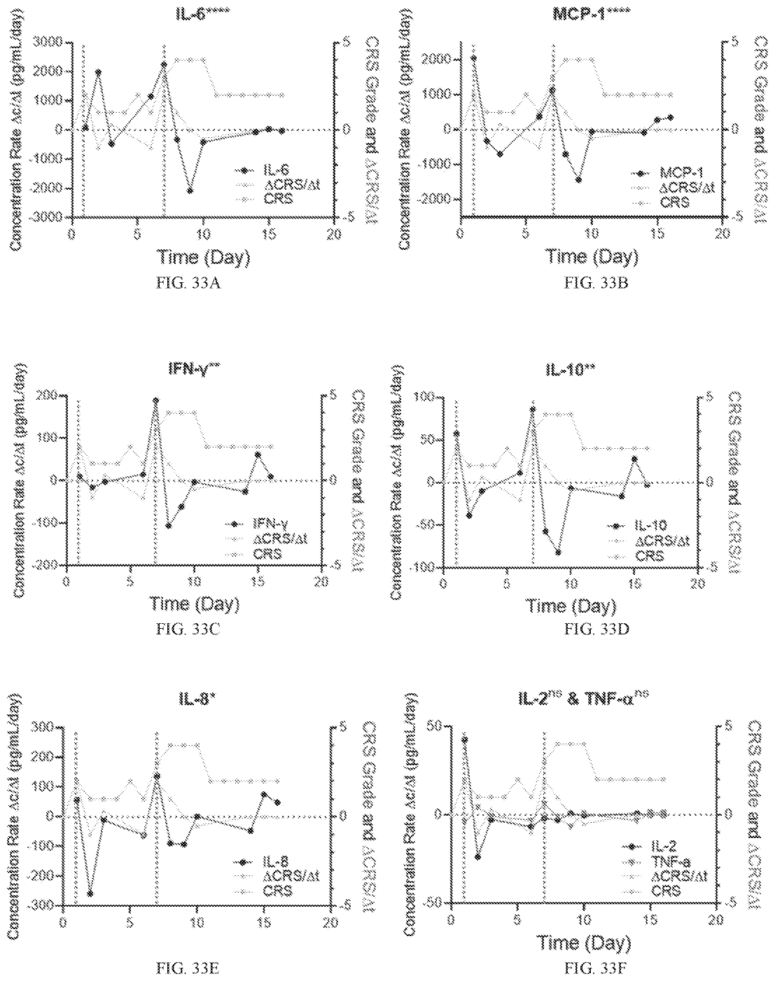

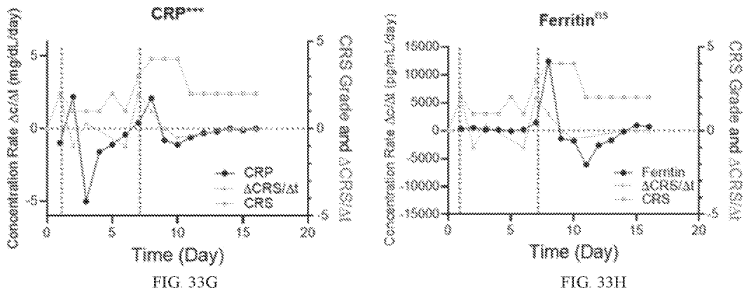

The responsiveness of each biomarker to the time evolution of CRS is depicted in FIG. 31E. FIG. 33 shows the longitudinal variations of the time rate of biomarker concentration change (Δc/Δt), the time rate of CRS score change (ΔCRS/Δt), and the CRS score for Patient 06 (grade 4 CRS). For the cytokines showing significant elevations upon CRS in FIG. 32 (IL-6, MCP-1, IFN-γ, IL-10, and IL-8), their time plots of Δc/Δt follow a similar trend to the time plot of ΔCRS/Δt. Furthermore, the peaks in the Δc/Δt plots for MCP-1, IFN-γ, IL-8 and IL-10 synchronously appeared with the peaks in the ΔCRS/Δt plot. Notably, sharp increase of IFN-γ represented by the second large peak in the Δc/Δt plot for IFN-γ accompanied a life-threatening complication for this patient. On the other hand, the Δc/Δt plots for the current clinical inflammatory surrogate markers, CRP and Ferritin, yielded no peak or a peak with a time (˜1 day) delay in response to the appearance of a peak of ΔCRS/Δt (FIGS. 33G-33H). A peak in the ΔCRS/Δt plot always preceded a significant deterioration of the CRS condition, represented by an increase of the CRS score.

The 2-step transient assay format of PEdELISA can maintain a linear relationship between the analyte concentration and the assay readout regardless of the snapshot acquisition timing. Additionally, the modeling predicted very well the minimum required incubation time for the desired detection limit, which guided the digital assay design. For IL-6, which is the primary mediator of CRS, the entire assay incubation time can be as short as 15 sec with a LOD of 25.9 pg/mL while maintaining a 4-order dynamic range up to 10 ng/mL. PEdELISA can continuously provide real-time data for blood samples freshly collected from human patients with a high time-resolution limited principally by blood sampling frequency (<24 hr over most of the course of the studies).

It is understood that the foregoing detailed description and accompanying examples are merely illustrative and are not to be taken as limitations upon the scope of the disclosure, which is defined solely by the appended claims and their equivalents.

Various changes and modifications to the disclosed embodiments will be apparent to those skilled in the art and may be made without departing from the spirit and scope thereof.

What is claimed is:

1. A method for detecting a molecule in a sample comprising:
contacting a sample with a capture agent specific for the molecule and a detection agent;
incubating the sample with the capture agent and detection agent to form a capture agent-molecule-detection agent complex, wherein the incubating is less than a time necessary for equilibrium conditions to be reached in formation of the complex; and
detecting said molecule.

2. The method of claim 1, further comprising the step of separating the capture agent and the capture agent-molecule-detection agent complex from remaining sample and unbound detection agent.

3. The method of claim 2, further comprising the step of isolating each capture agent and capture agent-molecule-detection agent complex into individual locations within a solid support.

4. The method of claim 3, wherein the detecting comprises determining the presence or absence of the capture agent and detection agent within each of the individual locations.

5. The method of any of claims 1-4, wherein the molecule is a polypeptide, a polysaccharide, a polynucleotide, a lipid, a metabolite, a drug, or a combination thereof.

6. The method of any of claims 1-5, wherein the molecule is a biomarker.

7. The method of any of claims 1-6, wherein said capture agent comprises a particle comprising a first probe configured to bind the molecule and the detection agent comprises a second probe configured to bind the molecule.

8. The method of claim 7, wherein the particle is a magnetic bead.

9. The method of claim 8, wherein the magnetic bead is a fluorescent magnetic bead.

10. The method of claim 8 or claim 9, wherein determining the presence or absence of the capture agent comprises detection of the magnetic bead.

11. The method of any of claims 7-10, wherein the first probe and the second probe are configured to bind to different locations within the molecule.

12. The method of any of claim 7-11, wherein the first probe and the second probe are independently selected from a protein, a peptide, a nucleic acid, a carbohydrate, a small molecule, and a ligand.

13. The method of any of claims 7-12, wherein the first probe is an antibody.

14. The method of any of claims 7-13, wherein the second probe is an antibody.

15. The method of any of claims 1-14, wherein the incubating is between 15 seconds and 45 minutes.

16. The method of any of claims 1-15, wherein the incubating is between 15 seconds and 600 seconds.

17. The method of any of claims 1-16, wherein the incubating is between 15 second and 300 seconds.

18. The method of any of claims 3-17, wherein the solid support is a microplate or microfluidic device.

19. The method of any of claims 1-18, wherein the detection agent further comprises a detection moiety selected from the group consisting of a dye, a radiolabel, an enzyme, and an enzyme substrate.

20. The method of claim 19, wherein the detection moiety is an enzyme.

21. The method of claim 19 or 20, wherein the enzyme is beta-galactosidase, alkaline phosphatase or horseradish peroxidase.

22. The method of any of claims 19-21, wherein the method further comprises
adding a labeling agent to the separated capture agent and the capture agent-molecule-detection agent complex; wherein the labeling agent reacts with the detection moiety to produce a reaction product,
wherein determining the presence or absence of the detection agent comprises measurement of the reaction product.

23. The method of any of claim 19, wherein determining the presence or absence of the detection agent comprises measurement of the detection moiety.

24. The method of any of claims 4-23, further comprising determining a fraction of locations comprising both the capture agent and the detection agent to locations comprising only the capture agent.

25. The method of claim 24, further comprising quantifying the concentration of the molecule based on the fraction of locations comprising both the capture agent and the detection agent to locations comprising only the capture agent.

26. The method of any of claims 1-25, wherein two or more molecules are detected with multiple pairs of capture agents and detection agents, each pair configured to uniquely bind one of the two or more molecules.

27. The method of any of claims 1-26, wherein the sample is a biological sample.

28. The method of any of claims 1-27, wherein the sample has a volume less than 100 uL.

29. The method of any of claims 1-28, wherein the sample has a volume between 1 and 25 uL.

30. A system for detecting a molecule in a sample comprising one or more or each of:
a capture agent comprising a particle coated with a first probe configured to bind the molecule;
a detection agent comprising a second probe configured to bind the molecule;
an incubator configured to incubate a sample with the capture agent and detection agent to form a capture agent-molecule-detection agent complex, for a time that is less than a time necessary for equilibrium conditions to be reached in formation of a complex between said capture agent, said detection agent, and said molecule;
a solid support;
a detector;
software configured to determine the presence or absence of the capture agent and the detection agent from the output of the detector; and
a sample.

31. The system of claim 30, wherein the particle is a magnetic bead.

32. The system of claim 31, wherein the magnetic bead is a fluorescent magnetic bead.

33. The system of any of claims 30-32, wherein the detection agent further comprises a detection moiety selected from the group consisting of a dye, a radiolabel, an enzyme, and an enzyme substrate.

34. The system of claims 30-33, further comprising a labeling agent.

35. The system of any of claims 30-34, wherein the solid support is a microplate or microfluidic device.

36. The system of any of claims 30-35, wherein the detector comprises an optical microscope, fluorescence microscope, a fluorometer, a spectrophotometer, a camera, or a combination thereof.

37. The system of any of claims 30-36, further comprising at least one additional pair of capture agent and detection agent.

38. A reaction mixture comprising:
a stopped incubation mixture of a sample comprising a molecule;
a capture agent;
a detection agent;
and a plurality of capture agent-molecule-detection agent complexes,
wherein the stopped mixture is stopped at a time less than a time necessary for equilibrium conditions to be reached in formation of the capture agent-molecule-detection agent complex.

39. The reaction mixture of claim 38, wherein the sample is a biological sample.

40. The reaction mixture of claim 38 or claim 39, wherein the capture agent comprises a particle comprising a first probe configured to bind the molecule.

41. The reaction mixture of claim 40, wherein the particle is a magnetic bead.

42. The reaction mixture of claim 41, wherein the magnetic bead is a fluorescent magnetic bead.

43. The reaction mixture of any of claims 40-42, wherein the first probe is selected from the group consisting of a protein, a peptide, a nucleic acid, a carbohydrate, a small molecule, a ligand and any combination thereof.

44. The reaction mixture of any of claims 38-43, wherein the detection agent comprises a second probe configured to bind the molecule.

45. The reaction mixture of claim 44, wherein the second probe is selected from the group consisting of a protein, a peptide, a nucleic acid, a carbohydrate, a small molecule, a ligand and any combination thereof.

46. A device for spatial-spectral encoding comprising:
a solid support comprising individual locations configured to isolate a molecule of interest;
a sample patterning component; and
a sample detection component.

47. The device of claim 46, wherein the sample patterning component and the sample detection component each comprise a plurality of parallel fluid handling channels.

48. The device of claim 47, where each fluid handling channel is independent from the adjacent fluid handling channel.

49. The device of claim 47 or 48, wherein each fluid handling channel is configured to receive a different fluid sample.

50. The device of any of claims 47-49, wherein each of the fluid handling channel comprises an individual inlet and outlet.

51. The device of any of claims 47-50, wherein each fluid handling channels is in fluid communication with a portion of the individual locations in the solid support.

52. The device of any of claims 47-51, wherein the parallel fluid handling channels of the sample patterning component are perpendicular to the parallel fluid handling channels of the sample detection component.

53. The device of any of claims 47-52, wherein the sample patterning component and a sample detection component are interchangeably attached to the solid support.

54. A system for spatial-spectral encoding comprising
the device of any of claims 46-53; and
a detector; and
software configured to determine the presence or absence of the capture agent and spatially identify the individual locations in the solid support.

55. The system of claim 54, further comprising a capture agent and a detection agent.

56. A method for spatial-spectral encoding a plurality of molecules of interest from at least one sample:
providing a device for spatial-spectral encoding comprising:
a solid support comprising spatially identifiable individual locations;
a sample patterning component; and
a sample detection component,
wherein the sample patterning component and the sample detection component each comprise a plurality of parallel fluid handling channels;
loading a capture agent pool into each fluid handling channel of the sample patterning component, wherein each capture agent pool is isolated to a portion of the individual locations in the solid support and each capture agent from the capture agent pool is isolated in an individual location;
contacting each sample with each capture agent comprising loading each sample into individual fluid handling channels of the sample detection component, wherein each sample is isolated to a portion of the individual locations with the solid support;
incubating each sample with the capture agent to form a capture agent-molecule complex;
contacting the capture agent-molecule complex with a detection agent to form a capture agent-molecule-detection agent complex;
detecting the presence or absence of capture agent and detection agent at each location with a detector; and
correlating the output of the detector with the presence or absence of at least one of the plurality of molecules of interest with software configured to spatially identify the individual locations in the solid support.
